SEQUENCE LISTING (275 sequences)

SEQ ID NO: 1 - protein, 124 aa, synthetic construct
  REGION 1..124: note = Synthetic
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVS

SEQ ID NO: 2 - protein, 112 aa, synthetic construct [sequence omitted]

SEQ ID NO: 3 - protein, 8 aa, synthetic construct
  REGION 1..8: note = Synthetic
GFTFRDYA

SEQ ID NO: 4 - protein, 8 aa, synthetic construct
  REGION 1..8: note = Synthetic
ISGSGGNT

SEQ ID NO: 5 - protein, 18 aa, synthetic construct
  REGION 1..18: note = Synthetic
AKDRLSITIRPRYYGLDV

SEQ ID NO: 6 - protein, 11 aa, synthetic construct
  REGION 1..11: note = Synthetic
QSLLYSIGYNY

SEQ ID NO: 7 - skipped

SEQ ID NO: 8 - protein, 9 aa, synthetic construct
  REGION 1..9: note = Synthetic
MQALQTPYT

SEQ ID NO: 9 - protein, 452 aa, synthetic construct
  REGION 1..452: note = Synthetic
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 219 aa, synthetic construct
  REGION 1..219: note = Synthetic
DIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 207 aa, synthetic construct
  REGION 1..207: note = Synthetic
MKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQH

SEQ ID NO: 12 - protein, 109 aa, synthetic construct
  REGION 1..109: note = Synthetic
EIVLTQSPGTLSLSPGERATLSCRASQSVSNSYLAWYQQKPGQAPRLLIFGASSRATGIPDRFSGSGSGTDFTLTISRLEPEDFAVYYCQQYGSSPPWTFGQGTKVEIK

SEQ ID NO: 13 - protein, 109 aa, synthetic construct
  REGION 1..109: note = Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPPWTFGQGTKVEIK

SEQ ID NO: 14 - protein, 109 aa, synthetic construct
  REGION 1..109: note = Synthetic
EIVLTQSPGTLSLSPGERATLSCRASQSVSSSYLAWYQQKPGQAPRLLIFGASSRAPGIPDRFSGSGSGTDFTLTISRLEPEDFAVYYCQQYGSSPPWTFGQGTKVEIK

SEQ ID NO: 15 - protein, 109 aa, synthetic construct
  REGION 1..109: note = Synthetic
EIVLTQSPGTLSLSPGERATLSCRASQSVSNSYLAWYQQKPGQAPRLLIYGASSRATGIPDRFSGSGSGTDFTLTISRLEPEDFAVYYCQQYGSSPPWTFGQGTKVEIK

SEQ ID NO: 16 - protein, 109 aa, synthetic construct
  REGION 1..109: note = Synthetic
EIVLTQSPGTLSLSPGERATLSCRASQSVSNSYLAWYQQKPGQAPRLLIFGASSRAPGIPDRFSGSGSGTDFTLTISRLEPEDFAVYYCQQYGSSPPWTFGQGTKVEIK

SEQ ID NO: 17 - protein, 109 aa, synthetic construct
  REGION 1..109: note = Synthetic
EIVLTQSPGTLSLSPGERATLSCRASQSVSSSYLAWYQQKPGQAPRLLIYGASSRAPGIPDRFSGSGSGTDFTLTISRLEPEDFAVYYCQQYGSSPPWTFGQGTKVEIK

SEQ ID NO: 18 - protein, 109 aa, synthetic construct
  REGION 1..109: note = Synthetic
EIVLTQSPGTLSLSPGERATLSCRASQSVSSSYLAWYQQKPGQAPRLLIFGASSRATGIPDRFSGSGSGTDFTLTISRLEPEDFAVYYCQQYDHSPPWTFGQGTKVEIK

SEQ ID NO: 19 - protein, 109 aa, synthetic construct
  REGION 1..109: note = Synthetic
EIVLTQSPGTLSLSPGERATLSCRASQSVSSSYLAWYQQKPGQAPRLLIFGASSRATGIPDRFSGSGSGTDFTLTISRLEPEDFAVYYCQQYGSSAGWTFGQGTKVEIK

SEQ ID NO: 20 - protein, 109 aa, synthetic construct
  REGION 1..109: note = Synthetic
EIVLTQSPGTLSLSPGERATLSCRASQSVSSSYLAWYQQKPGQAPRLLIFGASSRATGIPDRFSGSGSGTDFTLTISRLEPEDFAVYYCQQYDHSAGWTFGQGTKVEIK

SEQ ID NO: 21 - protein, 109 aa, synthetic construct
  REGION 1..109: note = Synthetic
EIVLTQSPGTLSLSPGERATLSCRASQSVSNSYLAWYQQKPGQAPRLLIFGASSRATGIPDRFSGSGSGTDFTLTISRLEPEDFAVYYCQQYDHSPPWTFGQGTKVEIK

SEQ ID NO: 22 - protein, 109 aa, synthetic construct
  REGION 1..109: note = Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYDHSPPWTFGQGTKVEIK

SEQ ID NO: 23 - protein, 109 aa, synthetic construct
  REGION 1..109: note = Synthetic
EIVLTQSPGTLSLSPGERATLSCRASQSVSSSYLAWYQQKPGQAPRLLIFGASSRAPGIPDRFSGSGSGTDFTLTISRLEPEDFAVYYCQQYDHSPPWTFGQGTKVEIK

SEQ ID NO: 24 - protein, 109 aa, synthetic construct
  REGION 1..109: note = Synthetic
EIVLTQSPGTLSLSPGERATLSCRASQSVSSSYLAWYQQKPGQAPRLLIYGASSRAPGIPDRFSGSGSGTDFTLTISRLEPEDFAVYYCQQYDHSAGWTFGQGTKVEIK

SEQ ID NO: 25 - protein, 109 aa, synthetic construct
  REGION 1..109: note = Synthetic
EIVLTQSPGTLSLSPGERATLSCRASQSVSNSYLAWYQQKPGQAPRLLIFGASSRAPGIPDRFSGSGSGTDFTLTISRLEPEDFAVYYCQQYDHSAGWTFGQGTKVEIK

SEQ ID NO: 26 - protein, 109 aa, synthetic construct
  REGION 1..109: note = Synthetic
EIVLTQSPGTLSLSPGERATLSCRASQSVSNSYLAWYQQKPGQAPRLLIYGASSRATGIPDRFSGSGSGTDFTLTISRLEPEDFAVYYCQQYDHSAGWTFGQGTKVEIK

SEQ ID NO: 27 - protein, 109 aa, synthetic construct
  REGION 1..109: note = Synthetic
EIVLTQSPGTLSLSPGERATLSCRASQSVSSSYLAWYQQKPGQAPRLLIFGASSRAPGIPDRFSGSGSGTDFTLTISRLEPEDFAVYYCQQYDHSAGWTFGQGTKVEIK

SEQ ID NO: 28 - protein, 109 aa, synthetic construct
  REGION 1..109: note = Synthetic
EIVLTQSPGTLSLSPGERATLSCRASQSVSSSYLAWYQQKPGQAPRLLIYGASSRATGIPDRFSGSGSGTDFTLTISRLEPEDFAVYYCQQYDHSAGWTFGQGTKVEIK

SEQ ID NO: 29 - protein, 109 aa, synthetic construct
  REGION 1..109: note = Synthetic
EIVLTQSPGTLSLSPGERATLSCRASQSVSNSYLAWYQQKPGQAPRLLIFGASSRATGIPDRFSGSGSGTDFTLTISRLEPEDFAVYYCQQYDHSAGWTFGQGTKVEIK

SEQ ID NO: 30 - protein, 109 aa, synthetic construct
  REGION 1..109: note = Synthetic
EIVLTQSPGTLSLSPGERATLSCRASQSVSSSYLAWYQQKPGQAPRLLIFGASSRATGIPDRFSGSGSGTDFTLTISRLEPEDFAVYYCQQYGSSPPWTFGQGTKVEIK

SEQ ID NO: 31 - protein, 109 aa, synthetic construct
  REGION 1..109: note = Synthetic
EIVLTQSPGTLSLSPGERATLSCRASQSVSNSYLAWYQQKPGQAPRLLIYGASSRAPGIPDRFSGSGSGTDFTLTISRLEPEDFAVYYCQQYDHSAGWTFGQGTKVEIK

SEQ ID NO: 32 - protein, 115 aa, synthetic construct
  REGION 1..115: note = Synthetic
EVQLVESGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 33 - protein, 115 aa, synthetic construct
  REGION 1..115: note = Synthetic
EVQLVQSGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 34 - protein, 115 aa, synthetic construct
  REGION 1..115: note = Synthetic
EVQLVQSGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 35 - protein, 115 aa, synthetic construct
  REGION 1..115: note = Synthetic
EVQLVQSGGGLVHPGGSLRLSCAGSGFTFSRNAMFWVRQAPGKGLEWVSGIGTGGATSYADSVKGRFTISRDNAKNSLYLQMNSLRAEDMAVYYCARGRYYFDYWGQGTLVTVSS

SEQ ID NO: 36 - protein, 115 aa, synthetic construct
  REGION 1..115: note = Synthetic
EVQLVQSGGGLVHPGGSLRLSCAGSGFTFSRNAMFWVRQAPGKGLEWVSGIGTGGATNYADSVKGRFTISRDNAKNSLYLQMNSLRAEDTAVYYCARGRYYFDYWGQGTLVTVSS

SEQ ID NO: 37 - protein, 115 aa, synthetic construct
  REGION 1..115: note = Synthetic
EVQLVESGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 38 - protein, 115 aa, synthetic construct
  REGION 1..115: note = Synthetic
EVQLVESGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 39 - protein, 115 aa, synthetic construct
  REGION 1..115: note = Synthetic
EVQLVQSGGGLVQPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 40 - protein, 115 aa, synthetic construct
  REGION 1..115: note = Synthetic
EVQLVQSGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFDYWGQGTLVTVSS

SEQ ID NO: 41 - protein, 115 aa, synthetic construct
  REGION 1..115: note = Synthetic
EVQLVQSGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 42 - protein, 115 aa, synthetic construct
  REGION 1..115: note = Synthetic
EVQLVESGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 43 - protein, 115 aa, synthetic construct
  REGION 1..115: note = Synthetic
EVQLVQSGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 44 - protein, 115 aa, synthetic construct
  REGION 1..115: note = Synthetic
EVQLVQSGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 45 - protein, 115 aa, synthetic construct
  REGION 1..115: note = Synthetic
EVQLVQSGGGLVHPGGSLRLSCAGSGFTFSRNAMFWVRQAPGKGLEWVSGIGTGGATSYADSVKGRFTISRDNAKNSLYLQMNSLRAEDMAVYYCARGRYYFPWWGQGTLVTVSS

SEQ ID NO: 46 - protein, 115 aa, synthetic construct
  REGION 1..115: note = Synthetic
EVQLVQSGGGLVHPGGSLRLSCAGSGFTFSRNAMFWVRQAPGKGLEWVSGIGTGGATNYADSVKGRFTISRDNAKNSLYLQMNSLRAEDTAVYYCARGRYYFPWWGQGTLVTVSS

SEQ ID NO: 47 - protein, 115 aa, synthetic construct
  REGION 1..115: note = Synthetic
EVQLVQSGGGLVHPGRSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 48 - protein, 115 aa, synthetic construct
  REGION 1..115: note = Synthetic
EVQLVQSGGGLVHPGGSLRLTCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 49 - protein, 115 aa, synthetic construct
  REGION 1..115: note = Synthetic
EVQLVQSGGGLVHPGGSLRLSCAGSGFTFSRNAMHWVRQAPGKGLEWVSGIGTGGATNYADSVKGRFTISRDNAKNSLYLQMNSLRAEDMAVYYCARGRYYFDYWGQGTLVTVSS

SEQ ID NO: 50 - protein, 115 aa, synthetic construct
  REGION 1..115: note = Synthetic
EVQLVQSGGGLVHPGGSLRLSCAGSGFTFSRNAMFWVRQAPGEGLEWVSGIGTGGATNYADSVKGRFTISRDNAKNSLYLQMNSLRAEDMAVYYCARGRYYFDYWGQGTLVTVSS

SEQ ID NO: 51 - protein, 115 aa, synthetic construct
  REGION 1..115: note = Synthetic
EVQLVQSGGGLVHPGGSLRLSCAGSGFTFSRNAMFWVRQAPGKGLEWVSGIGTGGATNYADSVKGRFTISRDEAKNSLYLQMNSLRAEDMAVYYCARGRYYFDYWGQGTLVTVSS

SEQ ID NO: 52 - protein, 115 aa, synthetic construct
  REGION 1..115: note = Synthetic
EVQLVQSGGGLVHPGGSLRLSCAGSGFTFSRNAMFWVRQAPGKGLEWVSGIGTGGATNYADSVKGRFTISRDNAKNSLYLQMNSLRAGDMAVYYCARGRYYFDYWGQGTLVTVSS

SEQ ID NO: 53 - protein, 115 aa, synthetic construct
  REGION 1..115: note = Synthetic
EVQLVQSGGGLVHPGGSLRLSCAGSGFTFDDYAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 54 - protein, 115 aa, synthetic construct
  REGION 1..115: note = Synthetic
EVQLVQSGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 55 - protein, 115 aa, synthetic construct
  REGION 1..115: note = Synthetic
EVQLVESGGGLVHPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 56 - protein, 115 aa, synthetic construct
  REGION 1..115: note = Synthetic
EVQLVESGGGLVQPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 57 - protein, 115 aa, synthetic construct
  REGION 1..115: note = Synthetic
EVQLVESGGGLVQPGGSLRLSCAASGFTFSRNAMFWVRQAPGKGLEWVSGIGTGGATNYADSVKGRFTISRDNAKNSLYLQMNSLRAEDTAVYYCARGRYYFPWWGQGTLVTVSS

SEQ ID NO: 58 - protein, 115 aa, synthetic construct
  REGION 1..115: note = Synthetic
EVQLVESGGGLVQPGGSLRLSCAASGFTFSRNAMFWVRQAPGKGLEWVSGIGTGGATSYADSVKGRFTISRDNAKNSLYLQMNSLRAEDMAVYYCARGRYYFPWWGQGTLVTVSS

SEQ ID NO: 59 - protein, 115 aa, synthetic construct
  REGION 1..115: note = Synthetic
EVQLVQSGGGLVHPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 60 - protein, 115 aa, synthetic construct
  REGION 1..115: note = Synthetic
EVQLVQSGGGLVQPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 61 - protein, 115 aa, synthetic construct
  REGION 1..115: note = Synthetic
EVQLVESGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 62 - protein, 115 aa, synthetic construct
  REGION 1..115: note = Synthetic
EVQLVQSGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 63 - protein, 115 aa, synthetic construct
  REGION 1..115: note = Synthetic
EVQLVESGGGLVQPGGSLRLSCAASGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 64 - protein, 115 aa, synthetic construct
  REGION 1..115: note = Synthetic
EVQLVESGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFDYWGQGTLVTVSS

SEQ ID NO: 65 - protein, 115 aa, synthetic construct
  REGION 1..115: note = Synthetic
EVQLVQSGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 66 - protein, 115 aa, synthetic construct
  REGION 1..115: note = Synthetic
EVQLVESGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 67 - protein, 116 aa, synthetic construct
  REGION 1..116: note = Synthetic
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLDYWGKGTLVTVSS

SEQ ID NO: 68 - protein, 111 aa, synthetic construct
  REGION 1..111: note = Synthetic
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSLSANYVFGTGTKLTVL

SEQ ID NO: 69 - protein, 116 aa, synthetic construct
  REGION 1..116: note = Synthetic
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLYNWGKGTLVTVSS

SEQ ID NO: 70 - protein, 111 aa, synthetic construct
  REGION 1..111: note = Synthetic
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSQPPNPLFGTGTKLTVL

SEQ ID NO: 71 - protein, 116 aa, synthetic construct
  REGION 1..116: note = Synthetic
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKLLKNPWGKGTLVTVSS

SEQ ID NO: 72 - protein, 111 aa, synthetic construct
  REGION 1..111: note = Synthetic
QSVLTQPPSVSAAPGQKVTISCSGGSSNIGNSYVSWYQQLPGTAPKLLIYDNNKRPSGIPDRFSGSKSGTSATLAITGLQTGDEADYYCGTWFGTPASNYVFGTGTKLTVL

SEQ ID NO: 73 - protein, 116 aa, synthetic construct; identical to SEQ ID NO: 69
  REGION 1..116: note = Synthetic

SEQ ID NO: 74 - protein, 111 aa, synthetic construct
  REGION 1..111: note = Synthetic
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSSPPQPIFGTGTKLTVL

SEQ ID NO: 75 - protein, 116 aa, synthetic construct
  REGION 1..116: note = Synthetic
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLYDWGKGTLVTVSS

SEQ ID NO: 76 - protein, 111 aa, synthetic construct; identical to SEQ ID NO: 74
  REGION 1..111: note = Synthetic

SEQ ID NO: 77 - protein, 116 aa, synthetic construct
  REGION 1..116: note = Synthetic
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 78 - protein, 111 aa, synthetic construct
  REGION 1..111: note = Synthetic
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTYHPIFGTGTKLTVL

SEQ ID NO: 79 - protein, 116 aa, synthetic construct
  REGION 1..116: note = Synthetic
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWWQYWGKGTLVTVSS

SEQ ID NO: 80 - protein, 111 aa, synthetic construct; identical to SEQ ID NO: 74
  REGION 1..111: note = Synthetic

SEQ ID NO: 81 - protein, 116 aa, synthetic construct; identical to SEQ ID NO: 79
  REGION 1..116: note = Synthetic

SEQ ID NO: 82 - protein, 111 aa, synthetic construct; identical to SEQ ID NO: 78
  REGION 1..111: note = Synthetic

SEQ ID NO: 83 - protein, 116 aa, synthetic construct; identical to SEQ ID NO: 69
  REGION 1..116: note = Synthetic

SEQ ID NO: 84 - protein, 111 aa, synthetic construct
  REGION 1..111: note = Synthetic
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTMYPLFGTGTKLTVL

SEQ ID NO: 85 - protein, 116 aa, synthetic construct; identical to SEQ ID NO: 75
  REGION 1..116: note = Synthetic

SEQ ID NO: 86 - protein, 111 aa, synthetic construct
  REGION 1..111: note = Synthetic
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTVLTPIFGTGTKLTVL

SEQ ID NO: 87 - protein, 116 aa, synthetic construct
  REGION 1..116: note = Synthetic
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWFYDWGKGTLVTVSS

SEQ ID NO: 88 - protein, 111 aa, synthetic construct
  REGION 1..111: note = Synthetic
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPSMIPLFGTGTKLTVL

SEQ ID NO: 89 - protein, 116 aa, synthetic construct; identical to SEQ ID NO: 87
  REGION 1..116: note = Synthetic

SEQ ID NO: 90 - protein, 111 aa, synthetic construct; identical to SEQ ID NO: 84
  REGION 1..111: note = Synthetic

SEQ ID NO: 91 - protein, 116 aa, synthetic construct; identical to SEQ ID NO: 75
  REGION 1..116: note = Synthetic

SEQ ID NO: 92 - protein, 111 aa, synthetic construct
  REGION 1..111: note = Synthetic
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTLQPLFGTGTKLTVL

SEQ ID NO: 93 - protein, 116 aa, synthetic construct; identical to SEQ ID NO: 69
  REGION 1..116: note = Synthetic

SEQ ID NO: 94 - protein, 111 aa, synthetic construct
  REGION 1..111: note = Synthetic
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPPTKPLFGTGTKLTVL

SEQ ID NO: 95 - protein, 116 aa, synthetic construct; identical to SEQ ID NO: 69
  REGION 1..116: note = Synthetic

SEQ ID NO: 96 - protein, 111 aa, synthetic construct
  REGION 1..111: note = Synthetic
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTHRHPLFGTGTKLTVL

SEQ ID NO: 97 - protein, 116 aa, synthetic construct; identical to SEQ ID NO: 69
  REGION 1..116: note = Synthetic

SEQ ID NO: 98 - protein, 111 aa, synthetic construct; identical to SEQ ID NO: 78
  REGION 1..111: note = Synthetic

SEQ ID NO: 99 - protein, 116 aa, synthetic construct
  REGION 1..116: note = Synthetic
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWWQHWGKGTLVTVSS

SEQ ID NO: 100 - protein, 111 aa, synthetic construct
  REGION 1..111: note = Synthetic
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPVDRPIFGTGTKLTVL

SEQ ID NO: 101 - protein, 116 aa, synthetic construct; identical to SEQ ID NO: 99
  REGION 1..116: note = Synthetic

SEQ ID NO: 102 - protein, 111 aa, synthetic construct
  REGION 1..111: note = Synthetic
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTPMPVFGTGTKLTVL

SEQ ID NO: 103 - protein, 116 aa, synthetic construct; identical to SEQ ID NO: 99
  REGION 1..116: note = Synthetic

SEQ ID NO: 104 - protein, 111 aa, synthetic construct; identical to SEQ ID NO: 78
  REGION 1..111: note = Synthetic

SEQ ID NO: 105 - protein, 116 aa, synthetic construct; identical to SEQ ID NO: 77
  REGION 1..116: note = Synthetic

SEQ ID NO: 106 - protein, 111 aa, synthetic construct
  REGION 1..111: note = Synthetic
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTVWEWPFGTGTKLTVL

SEQ ID NO: 107 - protein, 116 aa, synthetic construct
  REGION 1..116: note = Synthetic
QVQLVQSGAEVKKPGASVKVSCKASGYAFTSYYMHWARQAPGQGLEWMGIINPSGGSASYAQKFQGRVTMTRDTSTSTVYMELSSLRSEDTAVYYCARGKYWMYDWGKGTLVTVSS

SEQ ID NO: 108 - protein, 111 aa, synthetic construct
  REGION 1..111: note = Synthetic
QSVLTQPPSVSAAPGQKVTISCSGGSSNIGNSYVSWYQQLPGTAPKLLIYDNNKRPSGIPDRFSGSKSGTSATLAITGLQTGDEAVYFCGTWDTSTVWEWPFGTGTKLTVL

SEQ ID NO: 109 - protein, 116 aa, synthetic construct; identical to SEQ ID NO: 77
  REGION 1..116: note = Synthetic

SEQ ID NO: 110 - protein, 111 aa, synthetic construct
  REGION 1..111: note = Synthetic
QPVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 111 - protein, 116 aa, synthetic construct
  REGION 1..116: note = Synthetic
QVQLVQSGAEVRKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 112 - protein, 111 aa, synthetic construct
  REGION 1..111: note = Synthetic
QSVLTQPPSVSAAPGQKVTISCSGGSSNIGNNYVSWYQQLPGTAPKLLIYDNNKRPPGIPDRFSGSKSGTSATLAITGLQTGDEADYYCGTWDTSTVWEWPFGTGTKLTVL

SEQ ID NO: 113 - protein, 116 aa, synthetic construct
  REGION 1..116: note = Synthetic
QVQLVQSGAEVKKPGASVKVSCKASGYAFTSYYMHWARQAPGQGLEWMGIINPRGGSTSYAQKFQGRVTMTRDTSTSTVYMELSSLRSEDTAVYYCARGKYWMYDWGKGTLVTVSS

SEQ ID NO: 114 - protein, 111 aa, synthetic construct
  REGION 1..111: note = Synthetic
QSVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 115 - protein, 116 aa, synthetic construct
  REGION 1..116: note = Synthetic
QVQLVQSGAEVKKPGASVKVSCKASGYAFTSYYMHWARQAPGQGLEWMGIINPRGGSASYAQKFQGRVSMTRDTSTSTVYMELSSLRSEDTAVYYCARGKYWMYDWGKGTLVTVSS

SEQ ID NO: 116 - protein, 111 aa, synthetic construct
  REGION 1..111: note = Synthetic
QSVLTQPPSVSAAPGQKVTISCSGGSSNIGNSYVSWYQQLPGTAPKLLIYDNNKRPSGIPDRFSGSKSGTTATLAITGLQTGDEADYYCGTWVTSTVWEWPFGTGTKLTVL

SEQ ID NO: 117 - protein, 116 aa, synthetic construct; identical to SEQ ID NO: 77
  REGION 1..116: note = Synthetic

SEQ ID NO: 118 - protein, 111 aa, synthetic construct; identical to SEQ ID NO: 114
  REGION 1..111: note = Synthetic

SEQ ID NO: 119 - protein, 116 aa, synthetic construct
  REGION 1..116: note = Synthetic
QVQLVQSGAEVRKPGASVKVSCKASGYAFTSYYMHWARQAPGQGLEWMGIINPSGGSTSYAQKFQGRVTMTRDTSTSTVYMELSSLRPEDTAVYYCARGKYWMYDWGKGTQVTVSS

SEQ ID NO: 120 - protein, 111 aa, synthetic construct
  REGION 1..111: note = Synthetic
QSVLTQPPLVSAAPGQKVTISCSGGSSNIGNSYVSWYQRLPGTAPKLLIYDNNKRPSGIPDRFSGSKSGTSATLAITGLQTGDEADYYCGTWDTSTVWEWPFGTGTKLTVL

SEQ ID NO: 121 - protein, 116 aa, synthetic construct
  REGION 1..116: note = Synthetic
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGNGTLVTVSS

SEQ ID NO: 122 - protein, 111 aa, synthetic construct
  REGION 1..111: note = Synthetic
LPVLTQPPSVSAAPGQKVTISCSGGSSSIGNSYVSWYQQLPGAAPKLLIYDNNKRPSGIPDRFSGFRSGTSATLAITGLQTGDEADYYCGTWDTSPVWEWPFGTGTKLTVL

SEQ ID NO: 123 - protein, 116 aa, synthetic construct
  REGION 1..116: note = Synthetic
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTRVTVSS

SEQ ID NO: 124 - protein, 111 aa, synthetic construct
  REGION 1..111: note = Synthetic
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPVWEWPFGTGTKLTVL

SEQ ID NO: 125 - protein, 116 aa, synthetic construct; identical to SEQ ID NO: 77
  REGION 1..116: note = Synthetic

SEQ ID NO: 126 - protein, 111 aa, synthetic construct
  REGION 1..111: note = Synthetic
QSVLTQPPSVSAAPGQKVTISCSGGSSNIGNSYVSWYQRLPGAAPKLLIYDNNKRPSGIPDRFSGSKSGTSATLAITGLQTGDEADYYCGTWDTSTVWEWPFGTGTKLTVL

SEQ ID NO: 127 - protein, 116 aa, synthetic construct; identical to SEQ ID NO: 77
  REGION 1..116: note = Synthetic

SEQ ID NO: 128 - protein, 111 aa, synthetic construct
  REGION 1..111: note = Synthetic
QSVLTQPPSVSAAPGQKVTISCSGGSSSIGNSYVSWYQQLPGTAPKLLIYDNNKRPSGIPDRFSGSKSGTSATLAITGLQTGDEADYYCGTWATSPVWEWPFGTGTKLTVL

SEQ ID NO: 129 - protein, 116 aa, synthetic construct; identical to SEQ ID NO: 77
  REGION 1..116: note = Synthetic

SEQ ID NO: 130 - protein, 111 aa, synthetic construct
  REGION 1..111: note = Synthetic
QSVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTAWEWPFGTGTKLTVL

SEQ ID NO: 131 - protein, 116 aa, synthetic construct
  REGION 1..116: note = Synthetic
QVQLVQSGAEEKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 132 - protein, 111 aa, synthetic construct
  REGION 1..111: note = Synthetic
QSALTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 133 - protein, 116 aa, synthetic construct
  REGION 1..116: note = Synthetic
QVQLVQSGAEVKKPGASVKVSCKASGYAFTSYYMHWARQAPGQGLEWMGIINPSGGSTSYAQKFQGRVSMTRDTSTSTVYMELSSLRSEDTAVYYCARGKYWMYDWGKGTLVTVSS

SEQ ID NO: 134 - protein, 111 aa, synthetic construct; identical to SEQ ID NO: 114
  REGION 1..111: note = Synthetic

SEQ ID NO: 135 - protein, 116 aa, synthetic construct; identical to SEQ ID NO: 77
  REGION 1..116: note = Synthetic

SEQ ID NO: 136 - protein, 111 aa, synthetic construct; identical to SEQ ID NO: 124
  REGION 1..111: note = Synthetic

SEQ ID NO: 137 - protein, 116 aa, synthetic construct; identical to SEQ ID NO: 107
  REGION 1..116: note = Synthetic

SEQ ID NO: 138 - protein, 111 aa, synthetic construct
  REGION 1..111: note = Synthetic
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDSSTVWEWPFGTGTKLTVL

SEQ ID NO: 139 - protein, 116 aa, synthetic construct
  REGION 1..116: note = Synthetic
QVQLVQSGAEVKKPGASVKVSCKASGYAFTSYYMHWARQAPGQGLEWMGIINPRGGSTSYAQKFQGRVAMTRDTSTSTVYMELSSLRPEDTAVYYCARGKYWMYDWGKGTLVTVSS

SEQ ID NO: 140 - protein, 111 aa, synthetic construct
  REGION 1..111: note = Synthetic
QSVLTQPPSVSAAPGQKVTISCSGGGSSIGNSYVSWYQQLPGTAPKLLIYDNNKRPSGVPDRFSGSKSGTSATLAITGLQTGDEADYYCGTWDTSPVWEWPFGTGTKLTVL

SEQ ID NO: 141 - protein, 116 aa, synthetic construct; identical to SEQ ID NO: 107
  REGION 1..116: note = Synthetic

SEQ ID NO: 142 - protein, 111 aa, synthetic construct; identical to SEQ ID NO: 114
  REGION 1..111: note = Synthetic

SEQ ID NO: 143 - protein, 116 aa, synthetic construct; identical to SEQ ID NO: 113
  REGION 1..116: note = Synthetic

SEQ ID NO: 144 - protein, 111 aa, synthetic construct
  REGION 1..111: note = Synthetic
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTAWEWPFGTGTKLTVL

SEQ ID NO: 145 - protein, 116 aa, synthetic construct; identical to SEQ ID NO: 77
  REGION 1..116: note = Synthetic

SEQ ID NO: 146 - protein, 111 aa, synthetic construct; identical to SEQ ID NO: 138
  REGION 1..111: note = Synthetic

SEQ ID NO: 147 - protein, 116 aa, synthetic construct
  REGION 1..116: note = Synthetic
QVQLVQSGAEVRKPGASVKVSCKASGYAFTSYYMHWARQAPGQGLEWMGIINPSGGSTSYAQKFQGRVTMTRDTSTSTVYMELSSLRPEDTAVYYCARGKYWMYDWGKGTLVTVSG

SEQ ID NO: 148 - protein, 111 aa, synthetic construct
  REGION 1..111: note = Synthetic
QSVLTQPPSVSAAPGQKVTISCSGGSTNIGNSYVSWYQRLPGTAPKLLIYDNNKRPPGIPDRFSGSKSGTSATLAITGLQTGDEADYYCGTWDTSTVWEWPFGTGTKLTVL

SEQ ID NO: 149 - protein, 116 aa, synthetic construct
  REGION 1..116: note = Synthetic
QVQLVQSGAEVKKPGASVKVSCKASGYAFTSYYMHWARQAPGQGLEWVGIINPSGGSTSYAQKFQGRVTMTRDTSTSTVYMELSSLRSGDTAVYYCARGKYWMYDWGKGTLVTVSS

SEQ ID NO: 150 - protein, 111 aa, synthetic construct
  REGION 1..111: note = Synthetic
QAVLTQPPSVSAAPGQKVTISCSGGSSNIGNSYVSWYQRLPGAAPKLLIYDNNKRPSGIPDRFSGSKSGTSATLAITGLQTGDEADYYCGTWDTSTGWEWPFGTGTKLTVL

SEQ ID NO: 151 - protein, 116 aa, synthetic construct
  REGION 1..116: note = Synthetic
QVQLVQSGAEVKKPGASVKVSCKASGYAFTSYYMHWVRQAPGQGLEWMGIINPRGGSTSYAQKFQGRVTMTRDTSTSTVYMELSSLRSEDTAVYYCARGKYWMYDWGKGTLVTVSS

SEQ ID NO: 152 - protein, 111 aa, synthetic construct
  REGION 1..111: note = Synthetic
QSVLTQPPSVSAAPGQKVTISCSGGGSSIGNSYVSWYQQLPGTAPKLLIYDNNKRPSGIPDRFSGSKSGTSATLGITGLQTGDEADYYCGTWDTSPVWEWPFGTGTKLTVL

SEQ ID NO: 153 - protein, 116 aa, synthetic construct
  REGION 1..116: note = Synthetic
EVQLLESGGGLVQPGGSLRLSCAVSGFTFSNYAMSWVRQAPGKGLEWVSAISSGGGNIYYADSVKGRFTISRDNSKNTLYLQMNSLRAEDTAVYYCAKLRRYFDYWGQGTLVTVSS

SEQ ID NO: 154 - protein, 121 aa, synthetic construct
  REGION 1..121: note = Synthetic
EVQLLESGGGLVQPGGSLRLSCAASGFTFSDYAMSWVRQAPGKGLEWVSAISSGGSSIYYADSVKGRFTISRDNSKNTLHLQMNSLRAEDTAVYYCARGPQRSATAVFDYWGQGTLVTVSS

SEQ ID NO: 155 - protein, 127 aa, synthetic construct
  REGION 1..127: note = Synthetic
EVQLLESGGGLVQPGGSLRLSCAASGFTFSNYAMSWVRQAPGKGLEWVSWISPNSGNIYYADSVKGRFTISRDNSKNTLYLQMNSLRAEDTAVYYCARRPLSAAWSHSSYYNAMDVWGQGTLVTVSS

SEQ ID NO: 156 - protein, 116 aa, synthetic construct
  REGION 1..116: note = Synthetic
EVQLLESGGGLVQPGGSLRLSCAASGFTFSGYAMSWVRQAPGKGLEWVSLISHSGSNTYYADSVKGRFTISRDNSKNTLYLQMNSLRAEDTAVYYCARPHRAFDYWGQGTLVTVSS

SEQ ID NO: 157 - protein, 116 aa, synthetic construct
  REGION 1..116: note = Synthetic
EVQLLESGGGLVQPGGSLRLSCAASGFTFSNYAMSWVRQAPGKGLEWVSGISHGSGSIYYADSVKGRFTISRDNSKNTLYLQMNSLRAEDTAVYYCARPHRAFDYWGQGTLVTVSS

SEQ ID NO: 158 - protein, 116 aa, synthetic construct
  REGION 1..116: note = Synthetic
EVQLLESGGGLVQPGGSLRLSCAASGFTFSNYAMSWVRQAPGKGLEWVSGISHGNGSIYYADSVKGRFTISRDNSKNTLYLQMNSLRAEDTAVYYCAKTGRHFDYWGQGTLVTVSS

SEQ ID NO: 159 - protein, 116 aa, synthetic construct
  REGION 1..116: note = Synthetic
EVQLLESGGGLVQPGGSLRLSCAASGFTFSNYAMSWVRQAPGKGLEWVSSISPSGSSIYYADSVKGRFTISRDNSKNTLYLQMNSLRAEDTAVYYCARSYRAFDYWGQGTLVTVSS

SEQ ID NO: 160 - protein, 116 aa, synthetic construct
  REGION 1..116: note = Synthetic
EVQLLESGGGLVQPGGSLRLSCAASGFTFSNYAMSWVRQAPGKGLEWVSAISPSGGSIYYADSVKGRFTISRDNSKNTLYLQMNSLRAEDTAVYYCARAKRAFDYWGQGTLVTVSS

SEQ ID NO: 161 - protein, 116 aa, synthetic construct
  REGION 1..116: note = Synthetic
EVQLLESGGGLVQPGGSLRLSCAASGFTFSNYAMSWVRQAPGKGLEWVSAISPGSGSTYYADSVKGRFTISRDNSKNTLYLQMNSLRAEDTAVYYCAKFRRHFDYWGQGTLVTVSS

SEQ ID NO: 162 - protein, 116 aa, synthetic construct
  REGION 1..116: note = Synthetic
EVQLLESGGGLVQPGGSLRLSCAASGFTFSNYAMSWVRQAPGKGLEWVSAISSGGGNIYYADSVKGRFTISRDNSKNTLYLQMNSLRAEDTAVYYCARVHRAFDYWGQGTLVTVSS

SEQ ID NO: 163 - protein, 116 aa, synthetic construct
  REGION 1..116: note = Synthetic
EVQLLESGGGLVQPGGSLRLSCAASGFTFSNYAMSWVRQAPGKGLEWVSAITSSGRSIYYADSVKGRFTISRDNSKNTLYLQMNSLRAEDTAVYYCARVHRAFDYWGQGTLVTVSS

SEQ ID NO: 164 - protein, 116 aa, synthetic construct
  REGION 1..116: note = Synthetic
EVQLLESGGGLVQPGGSLRLSCAASGFTFSNYAMSWVRQAPGKGLEWVSAITSSGANIYYADSVKGRFTISRDNSKNTLYLQMNSLRAEDTAVYYCARVHRAFDYWGQGTLVTVSS

SEQ ID NO: 165 - protein, 116 aa, synthetic construct
  REGION 1..116: note = Synthetic
EVQLLESGGGLVQPGGSLRLSCAASGFTFSNYAMSWVRQAPGKGLEWVSAITSSGGNIYYADSVKGRFTISRDNSKNTLYLQMNSLRAEDTAVYYCARVHRAFDYWGQGTLVTVSS

SEQ ID NO: 166 - protein, 116 aa, synthetic construct
  REGION 1..116: note = Synthetic
EVQLLESGGGLVQPGGSLRLSCAASGFTFSNYAMSWVRQAPGKGLEWVSAITAGGGSIYYADSVKGRFTISRDNSKNTLYLQMNSLRAEDTAVYYCARVHRAFDYWGQGTLVTVSS

SEQ ID NO: 167 - protein, 116 aa, synthetic construct
  REGION 1..116: note = Synthetic
EVQLLESGGGLVQPGGSLRLSCAASGFTFSRHAMAWVRQAPGKGLEWVSAITSSGRSIYYADSVKGRFTISRDNSKNTLYLQMNSLRAEDTAVYYCARVHRAFDYWGQGTLVTVSS

SEQ ID NO: 168 - protein, 110 aa, synthetic construct
  REGION 1..110: note = Synthetic
QSVLTQPPSASGTPGQRVTISCSGSSSNIGNNYVNWYQQLPGTAPKLLIYDNSHRPSGVPDRFSGSKSGTSASLAISGLRSEDEADYYCGTWDASLSAYVFGGGTKLTVL

SEQ ID NO: 169 - protein, 110 aa, synthetic construct
  REGION 1..110: note = Synthetic
QSVLTQPPSASGTPGQRVTISCSGSSSNIGNNNVSWYQQLPGTAPKLLIYANSKRPSGVPDRFSGSKSGTSASLAISGLRSEDEADYYCGSWDDSLSAYVFGGGTKLTVL

SEQ ID NO: 170 - protein, 110 aa, synthetic construct
  REGION 1..110: note = Synthetic
QSVLTQPPSAPGTPGQRVTISCTGSSSNIGSNSVNWYQQLPGTAPKLLIYDDSHRPSGVPDRFSGSKSGTSASLAISGLRSEDEADYYCDAWDSSLSAYVFGGGTKLTVL

SEQ ID NO: 171 - protein, 110 aa, synthetic construct
  REGION 1..110: note = Synthetic
QSVLTQPPSASGTPGQRVTLSCTGSSSNIGSNYVSWYQQLPGTAPKLLIYADSQRPSGVPDRFSGSKSGTSASLAISGLRSEDEADYYCGTWDDSLSGYVFGGGTKLTVL

SEQ ID NO: 172 - protein, 110 aa, synthetic construct
  REGION 1..110: note = Synthetic
QSVLTQPPSASGTPGQRVTISCSSSSSNIGSNYVSWYQQLPGTAPKLLIYSDSHRPSGVPDRFSGSKSGTSASLAISGLRSEDEADYYCGSWDYSLSAYVFGGGTKLTVL

SEQ ID NO: 173 - protein, 110 aa, synthetic construct
  REGION 1..110: note = Synthetic
QSVLTQPPSASGTPGQRVTISCTGSSSNIGNNTVSWYQQLPGTAPKLLIYDNSHRPSGVPDRFSGSKSGTSASLAISGLQSEDEADYYCGSWDYSLSAYVFGGGTKLTVL

SEQ ID NO: 174 - protein, 110 aa, synthetic construct
  REGION 1..110: note = Synthetic
QSVLTQPPSASGTPGQRVTISCTGSSSNIGNNDVNWYQQLPGTAPKLLIYYDSQRPSGVPDRFSGSKSGTSASLAISGLRSEDEADYYCATWDASLSAYVFGGGTKLTVL

SEQ ID NO: 175 - protein, 110 aa, synthetic construct
  REGION 1..110: note = Synthetic
QSVLTQPPSASGTPGQRVTISCSGSSSNIGSNAVNWYQQLPGTAPKLLIYYDNQRPSGVPDRFSGSKSGTSASLAISGLRSEDEADYYCGTWDDSLNGYVFGGGTKLTVL

SEQ ID NO: 176 - protein, 110 aa, synthetic construct
  REGION 1..110: note = Synthetic
QSVLTQPPSASGTPGQRVTISCSGSSSNIGNNAVTWYQQLPGTAPKLLIYDDSHRPSGVPDRFSGSKSGTSASLAISGLRSEDEADYYCGSWDYSLSAYVFGGGTKLTVL

SEQ ID NO: 177 - protein, 110 aa, synthetic construct
  REGION 1..110: note = Synthetic
QSVLTQPPSASGTPGQRVTISCSGSSSNIGSNTFNWYQQLPGTAPKLLIYADSHRPSGVPDRFSGSKSGTSASLAISGLRSEDEADYYCGTWDYSLSGYVLGGGTKLTVL

SEQ ID NO: 178 - protein, 110 aa, synthetic construct; identical to SEQ ID NO: 177
  REGION 1..110: note = Synthetic

SEQ ID NO: 179 - protein, 110 aa, synthetic construct
  REGION 1..110: note = Synthetic
QSVLTQPPSASGTPGQRVTISCSGSSSNIGSNTFNWYQQLPGTAPKLLIYADSHRPSGVPDRFSGSKSGTSASLAISGLRSEDEADYYCGTWDYSLRGYVLGGGTKLTVL

SEQ ID NO: 180 - protein, 110 aa, synthetic construct
  REGION 1..110: note = Synthetic
QSVLTQPPSASGTPGQRVTISCSGSSSNIGSNTFNWYQQLPGTAPKLLIYADSHRPSGVPDRFSGSKSGTSASLAISGLRSEDEADYYCGYWDYSLSGYVLGGGTKLTVL

SEQ ID NO: 181 - protein, 110 aa, synthetic construct; identical to SEQ ID NO: 177
  REGION 1..110: note = Synthetic

SEQ ID NO: 182 - protein, 111 aa, synthetic construct
  REGION 1..111: note = Synthetic
QSVLTQPPSASGTPGQRVTISCSGSSANSRTDGFNWYQQLPGTAPKLLIYADSHRPSGVPDRFSGSKSGTSASLAISGLRSEDEADYYCGTWDYSLSGYVLGGGTKLTVLG

SEQ ID NO: 183 - protein, 111 aa, synthetic construct
  REGION 1..111: note = Synthetic
QSVLTQPPSASGTPGQRVTISCSGSAQFGSRDNFNWYQQLPGTAPKLLIYADSHRPSGVPDRFSGSKSGTSASLAISGLRSEDEADYYCGTWDYSLSGYVLGGGTKLTVLG

SEQ ID NO: 184 - protein, 111 aa, synthetic construct
  REGION 1..111: note = Synthetic
QSVLTQPPSASGTPGQRVTISCSGSTKQMHNYQFNWYQQLPGTAPKLLIYADSHRPSGVPDRFSGSKSGTSASLAISGLRSEDEADYYCGTWDYSLSGYVLGGGTKLTVLG

SEQ ID NO: 185 - protein, 111 aa, synthetic construct
  REGION 1..111: note = Synthetic
QSVLTQPPSASGTPGQRVTISCSGSLLRGENLQFNWYQQLPGTAPKLLIYADSHRPSGVPDRFSGSKSGTSASLAISGLRSEDEADYYCGTWDYSLSGYVLGGGTKLTVLG

SEQ ID NO: 186 - protein, 111 aa, synthetic construct
  REGION 1..111: note = Synthetic
QSVLTQPPSASGTPGQRVTISCSGSPLFPDSGSFNWYQQLPGTAPKLLIYADSHRPSGVPDRFSGSKSGTSASLAISGLRSEDEADYYCGTWDYSLSGYVLGGGTKLTVLG

SEQ ID NO: 187 - protein, 111 aa, synthetic construct
  REGION 1..111: note = Synthetic
QSVLTQPPSASGTPGQRVTISCSGSAALDLSPSFNWYQQLPGTAPKLLIYADSHRPSGVPDRFSGSKSGTSASLAISGLRSEDEADYYCGTWDYSLSGYVLGGGTKLTVLG

SEQ ID NO: 188 - protein, 117 aa, synthetic construct
  REGION 1..117: note = Synthetic
QVQLVQSGAEVKKPGASVKVSCKASGYTFTNYGISWVRQAPGQGLEWMGWISVYNGKTNYAQKLQGRVTMTTDTSTTTAYMEMRSLRSDDTAVYYCARGSGYDLDYWGQGTLVSVSS

SEQ ID NO: 189 - protein, 128 aa, synthetic construct
  REGION 1..128: note = Synthetic
EVQLVESGGGLVQPGGSLRLSCAASGFTFSSFWMTWVRQAPGKGLEWVANIKQDGSEKYYVDSVKGRFTISRDNAKNSLYLQMNSLRAEDTAVYYCARDPGRTMVRGGIRYYYGMDVWGQGTTVTVSS

SEQ ID NO: 190 - protein, 122 aa, synthetic construct
  REGION 1..122: note = Synthetic
EVKLAESGGGLVQPGGSLRLSCAASGFTFSSHWMNWVRQAPGKGLEWVANIKQDGSDKYYVDSVKGRFTISRDNAKNSLYLQLNSLIAEDTAVYYCARDRGVRPPRGAFDIWGQGTMVTVSS

SEQ ID NO: 191 - protein, 128 aa, synthetic construct
  REGION 1..128: note = Synthetic
QVQLVQSGAEVKKPGASVKVSCKASGYTFNSYGISWVRQAPGQGLEWMGWIRTYNGNTNYAQKLQGRVTMTTDTSTSTAYMELRSLRSDDTAVYYCARDEARIVVAGTTPYYYGMDVWGQGTTVTVSS

SEQ ID NO: 192 - protein, 117 aa, synthetic construct
  REGION 1..117: note = Synthetic
QVQLVESGGGLVQPGGSLRLSCAVSGFTISDHYMSWIRQAPGKGLEWISYISSSGSKIYYADSVKGRFTISRDNAKNSLFLQMNSLRAEDTAVYYCARTRQLVGDYWGQGTLVTVSS

SEQ ID NO: 193 - protein, 124 aa, synthetic construct
  REGION 1..124: note = Synthetic
EVQLVESGGGLVQPGRSLRLSCAASGFTFDNYAMHWVRQAPGKGLEWVSGIRWNSGSIGYADSVKGRFTISRDNAKNSLYLQMNSLRAEDTALYYCAKEGGYSGYRPGPFFDYWGQGTLVTVSS

SEQ ID NO: 194 - protein, 117 aa, synthetic construct
  REGION 1..117: note = Synthetic
QVQLVQSGAEVKKPGASVKVSCKASGYTFTNYGISWVRQAPGQGLEWMGWISVYNGHTNYAQKLQGRVTMTTDTSTSTAYMELRSLRSDDTAVYYCARGSGYDFDSWGQGTLVTVSS

SEQ ID NO: 195 - protein, 116 aa, synthetic construct
  REGION 1..116: note = Synthetic
QVQLVQSGAEVKKPGASVKVSCKASRYTFTSYDINWVRQATGQGLEWMGWMNPNSGNTGYAQKFQGRVTMTRNTSTSTAYMELSSLRSEDTAVYYCARVRRFFDYWGQGTLVTVSS

SEQ ID NO: 196 - protein, 117 aa, synthetic construct
  REGION 1..117: note = Synthetic
QVQLVQSGPEVKKPGASVKVSCKASGYTFTNYGISWVRQAPGQGLEWMGWISVYNGNINYAQKLQGRVTMTTDTSTSTAYMDLRSLRSDDTAVYYCARGSGYDFDYWGQGTLVTVSS

SEQ ID NO: 197 - protein, 122 aa, synthetic construct
  REGION 1..122: note = Synthetic
QVQLVQSGAEVKKPGASVKVSCKDSAYTFNRYGISWVRQAPGQGLEWMGWISAYTGNTVYAQKLQGRVTMTTDNSTSTAYMELRSLRSDDTAVYYCARDKSIFGVVRGFDYWGQGTLVTVSS

SEQ ID NO: 198 - protein, 107 aa, synthetic construct
  REGION 1..107: note = Synthetic
AIQMTQSPSSLSASVGDRVTITCRASQGIRNALGWYQQKPGKAPKLLIYAASSLQSGVPSRFSGSGSGTDFTLTFSSLQPEDFATYYCLQDFNYPYTFGQGTKLEIK

SEQ ID NO: 199 - protein, 107 aa, synthetic construct
  REGION 1..107: note = Synthetic
DIQMTQSPSSVSASVGDRVTISCRASQGVSSWLAWYQQKPGNAPKLLISAASSIQSGVPSRFSGSGSGTDFTLTISSLQPEDFATYYCQQANSFPLTFGGGTKVEIK

SEQ ID NO: 200 - protein, 107 aa, synthetic construct
  REGION 1..107: note = Synthetic
DIQMTQSPSSVSASVGDRVTITCRASQGISSWLAWYQQKPGKAPKLLIYAASSFQSGVPSRFSGSGSGTDFTLTISSLQPEDFATYFCQQANSFPLTFGGGTTVEIK

SEQ ID NO: 201 - protein, 107 aa, synthetic construct
  REGION 1..107: note = Synthetic
DIQMTQSPSSVSASVGDRVTITCRASQDISIWLAWYQQSPGKAPKLLINVASRLQSGVPSRFSGSGSGTDFTLTINSLQPEDFVTYYCQQANSFPITFGQGTRLATK

SEQ ID NO: 202 - protein, 107 aa, synthetic construct
  REGION 1..107: note = Synthetic
DIQLTQSPSFLSASVGDRVTITCWASQGISSYLAWYQQKPGKAPKLLIFAASTLQSGVPSRFSGSGSGTEFTLTISSLQPEDFATYYCQQLNSYPLTFGGGTKVEIR

SEQ ID NO: 203 - protein, 107 aa, synthetic construct
  REGION 1..107: note = Synthetic
EIVMTQSPATLSVSPGERATLSCRASQSVNYNLAWYQHKPGQAPRLLIYGASTRATGIPARFSGSGSGTEFTLTISSLQSEDFAVYYCQQYNNWPLTFGGGTKVEIK

SEQ ID NO: 204 - protein, 107 aa, synthetic construct
  REGION 1..107: note = Synthetic
AIQMTQSSSSLSASVGDRVTITCRASQAIRNALGWYQQKPGKAPKVLIYAASSLQSGIPSRFSGSGSGTDFTLTISSLQPEDFATYYCLQDYDYPYTFGQGTKLEIK

SEQ ID NO: 205 - protein, 107 aa, synthetic construct
  REGION 1..107: note = Synthetic
DIQLTQSPSFLSASVGDRVTITCWASQGIISYLAWYQQKPGKAPKLLIYAASTLHSGVPSRFSGSGSGTEFTLTISSLQPEDFATYYCHQLKSYPITFGQGTRLEIK

SEQ ID NO: 206 - protein, 107 aa, synthetic construct
  REGION 1..107: note = Synthetic
AIQMTQSPSSLSASVGDRVTITCRASQDIRNALGWYQQKPGKAPKLLIYAASSLQSGVPSRFSGSASGTDFTLTISSLQPEDFAAYYCLQDYNYPYTFGQGTKLEIK

SEQ ID NO: 207 - protein, 107 aa, synthetic construct
  REGION 1..107: note = Synthetic
EIVMTQSPVTLSLSPGERATLPCRASQSVSSSLAWYQQKAGQSPRLLIYGASTRATGIPARFSGSGSGTEFTLTISNLQSEDFAVYYCQQYNNWPLTFGGGTKVEIK

SEQ ID NO: 208 - protein, 120 aa, synthetic construct
  REGION 1..120: note = Synthetic
EVQLLESGGGLVQPGGSLRLSCAASGFTLSSYAMHWVRQAPGKGLEYVSGISSNGGSTYYANSVKGRFTISRDNPKNTLFLQMSSLRAEDTAVYYCVRVKVGYRGGMDVWGQGTTVTVSS

SEQ ID NO: 209 - protein, 120 aa, synthetic construct
  REGION 1..120: note = Synthetic
EVQLLESGGGLVQPGGSLRLSCAASGFTLSSYAMHWVRQAPGKGLEYVSGISPSGSSTYYANSVKGRFTISRDNPKNTLFLQMSSLRAEDTAVYYCVRSKVRYRGGMDVWGQGTTVTVSS

SEQ ID NO: 210 - protein, 120 aa, synthetic construct
  REGION 1..120: note = Synthetic
EVQLLESGGGLVQPGGSLRLSCAASGFTLSSYAMHWVRQAPGKGLEYVSGISPSGVSTYYANSVKGRFTISRDNPKNTLFLQMSSLRAEDTAVYYCVRVKVKYRGGMDVWGQGTTVTVSS

SEQ ID NO: 211 - protein, 120 aa, synthetic construct
  REGION 1..120: note = Synthetic
EVQLLESGGGLVQPGGSLRLSCAASGFTLSSYAMHWVRQAPGKGLEYVSGISPTSGSTYYANSVKGRFTISRDNPKNTLFLQMSSLRAEDTAVYYCVRVKVRYRGGMDVWGQGTTVTVSS

SEQ ID NO: 212 - protein, 120 aa, synthetic construct
  REGION 1..120: note = Synthetic
EVQLLESGGGLVQPGGSLRLSCAASGFTLSSYAMHWVRQAPGKGLEYVSGISPTGTSTYYANSVKGRFTISRDNPKNTLFLQMSSLRAEDTAVYYCVRVKGAYRGGMDVWGQGTTVTVSS

SEQ ID NO: 213 - protein, 120 aa, synthetic construct
  REGION 1..120: note = Synthetic
EVQLLESGGGLVQPGGSLRLSCAASGFTLSSYAMHWVRQAPGKGLEYVSGISSSGSSTYYANSVKGRFTISRDNPKNTLFLQMSSLRAEDTAVYYCVRVKVAYRGGMDVWGQGTTVTVSS

SEQ ID NO: 214 - protein, 120 aa, synthetic construct
  REGION 1..120: note = Synthetic
EVQLLESGGGLVQPGGSLRLSCAASGFTLSSYAMHWVRQAPGKGLEYVSGISPSSTSTYYANSVKGRFTISRDNPKNTLFLQMSSLRAEDTAVYYCVRVKVLYRGGMDVWGQGTTVTVSS

SEQ ID NO: 215 - protein, 120 aa, synthetic construct
  REGION 1..120: note = Synthetic
EVQLLESGGGLVQPGGSLRLSCAASGFTLSSYAMHWVRQAPGKGLEYVSGISPSSASTYYANSVKGRFTISRDNPKNTLFLQMSSLRAEDTAVYYCVRVKSKYRGGMDVWGQGTTVTVSS

SEQ ID NO: 216 - protein, 120 aa, synthetic construct
  REGION 1..120: note = Synthetic
EVQLLESGGGLVQPGGSLRLSCAASGFTLSSYAMHWVRQAPGKGLEYVSGISGNSASTYYANSVKGRFTISRDNPKNTLFLQMSSLRAEDTAVYYCVRVKLKYRGGMDVWGQGTTVTVSS

SEQ ID NO: 217 - protein, 120 aa, synthetic construct
  REGION 1..120: note = Synthetic
EVQLLESGGGLVQPGGSLRLSCAASGFTLSSYAMHWVRQAPGKGLEYVSGISHSGTSTYYANSVKGRFTISRDNPKNTLFLQMSSLRAEDTAVYYCVRVRVLYRGGMDVWGQGTTVTVSS

SEQ ID NO: 218 - protein, 120 aa, synthetic construct; identical to SEQ ID NO: 210
  REGION 1..120: note = Synthetic

SEQ ID NO: 219 - protein, 120 aa, synthetic construct
  REGION 1..120: note = Synthetic
EVQLLESGGGLVQPGGSLRLSCAASGFTLSSYAMHWVRQAPGKGLEYVSGISSNGGSTYYANSVKGRFTISRDNPKNTLFLQMSSLRAEDTAVYYCVRVFVRYRGGMDVWGQGTTVTVSS

SEQ ID NO: 220 - protein, 120 aa, synthetic construct
  REGION 1..120: note = Synthetic
EVQLLESGGGLVQPGGSLRLSCAASGFTLSSYAMHWVRQAPGKGLEYVSGISPTSASTYYANSVKGRFTISRDNPKNTLFLQMSSLRAEDTAVYYCVRVKGRYRGGMDVWGQGTTVTVSS

SEQ ID NO: 221 - protein, 120 aa, synthetic construct
  REGION 1..120: note = Synthetic
EVQLLESGGGLVQPGGSLRLSCAASGFTLSSYAMHWVRQAPGKGLEYVSGISPTGGSTYYANSVKGRFTISRDNPKNTLFLQMSSLRAEDTAVYYCVRVKGRYRGGMDVWGQGTTVTVSS

SEQ ID NO: 222 - protein, 120 aa, synthetic construct
  REGION 1..120: note = Synthetic
EVQLLESGGGLVQPGGSLRLSCAASGFTLSSYAMHWVRQAPGKGLEYVSGISHSGNSTYYANSVKGRFTISRDNPKNTLFLQMSSLRAEDTAVYYCVRVKRRYRGGMDVWGQGTTVTVSS

SEQ ID NO: 223 - protein, 120 aa, synthetic construct
  REGION 1..120: note = Synthetic
EVQLLESGGGLVQPGGSLRLSCAASGFTLSSYAMHWVRQAPGKGLEYVSGISPSSNSTYYANSVKGRFTISRDNPKNTLFLQMSSLRAEDTAVYYCVRVKVRYRGGMDVWGQGTTVTVSS

SEQ ID NO: 224 - protein, 120 aa, synthetic construct
  REGION 1..120: note = Synthetic
EVQLLESGGGLVQPGGSLRLSCAASGFTLSSYAMHWVRQAPGKGLEYVSGISSSGSSTYYANSVKGRFTISRDNPKNTLFLQMSSLRAEDTAVYYCVRVKPAYRGGMDVWGQGTTVTVSS

SEQ ID NO: 225 - protein, 120 aa, synthetic construct
  REGION 1..120: note = Synthetic
EVQLLESGGGLVQPGGSLRLSCAASGFTLSSYAMHWVRQAPGKGLEYVSGISYSSASTYYANSVKGRFTISRDNPKNTLFLQMSSLRAEDTAVYYCVRVKVRYRGGMDVWGQGTTVTVSS

SEQ ID NO: 226 - protein, 108 aa, synthetic construct
  REGION 1..108: note = Synthetic
ETTLTQSPDTLPLSPGDRASLSCRASQSVSSAYLAWYQQKPGQAPRLLIYGTSRRATGVPGRFSGSGSGTDFTLTISRLEPEDFAVYYCQLYGSSSVTFGQGTKLEIK

SEQ ID NO: 227 - protein, 108 aa, synthetic construct
  REGION 1..108: note = Synthetic
EIVLTQSPGTLSLSPGERATLSCRASQGISSAYLAWYQQKPGQAPRLLIYGTSRRATGIPDRFSGSGSGTDFTLTISRLEPEDFAVYYCQLYGATSVTFGQGTKLEIK

SEQ ID NO: 228 - protein, 108 aa, synthetic construct; identical to SEQ ID NO: 227
  REGION 1..108: note = Synthetic

SEQ ID NO: 229 - protein, 108 aa, synthetic construct
  REGION 1..108: note = Synthetic
EIVLTQSPGTLSLSPGERATLSCRASQSVSSAYLAWYQQKPGQAPRLLIYGTSRRATGIPDRFSGSGSGTDFTLTISRLEPEDFAVYYCQLYGASSVTFGQGTKLEIK

SEQ ID NO: 230 - protein, 108 aa, synthetic construct; identical to SEQ ID NO: 229
  REGION 1..108: note = Synthetic

SEQ ID NO: 231 - protein, 108 aa, synthetic construct; identical to SEQ ID NO: 227
  REGION 1..108: note = Synthetic

SEQ ID NO: 232 - protein, 108 aa, synthetic construct; identical to SEQ ID NO: 229
  REGION 1..108: note = Synthetic

SEQ ID NO: 233 - protein, 108 aa, synthetic construct
  REGION 1..108: note = Synthetic
EIVLTQSPGTLSLSPGERATLSCRASQSISTAYLAWYQQKPGQAPRLLIYGTSRRATGIPDRFSGSGSGTDFTLTISRLEPEDFAVYYCQLYGASSVTFGQGTKLEIK

SEQ ID NO: 234 - protein, 108 aa, synthetic construct
  REGION 1..108: note = Synthetic
EIVLTQSPGTLSLSPGERATLSCRASQDISSAYLAWYQQKPGQAPRLLIYGTSRRATGIPDRFSGSGSGTDFTLTISRLEPEDFAVYYCQLYGATSVTFGQGTKLEIK

SEQ ID NO: 235 - protein, 108 aa, synthetic construct
  REGION 1..108: note = Synthetic
EIVLTQSPGTLSLSPGERATLSCRASQDVSSAYLAWYQQKPGQAPRLLIYGTSRRATGIPDRFSGSGSGTDFTLTISRLEPEDFAVYYCQLYGATSVTFGQGTKLEIK

SEQ ID NO: 236 - protein, 108 aa, synthetic construct
  REGION 1..108: note = Synthetic
EIVLTQSPGTLSLSPGERATLSCRASQNISTAYLAWYQQKPGQAPRLLIYGTSRRATGIPDRFSGSGSGTDFTLTISRLEPEDFAVYYCQLYGATSVTFGQGTKLEIK

SEQ ID NO: 237 - protein, 108 aa, synthetic construct
  REGION 1..108: note = Synthetic
EIVLTQSPGTLSLSPGERATLSCRASQDASNAYLAWYQQKPGQAPRLLIYGTSRRATGIPDRFSGSGSGTDFTLTISRLEPEDFAVYYCQLYGSSSVTFGQGTKLEIK

SEQ ID NO: 238 - protein, 108 aa, synthetic construct
  REGION 1..108: note = Synthetic
EIVLTQSPGTLSLSPGERATLSCRASQGVSSAYLAWYQQKPGQAPRLLIYGTSRRATGIPDRFSGSGSGTDFTLTISRLEPEDFAVYYCQLYGRSSVTFGQGTKLEIK

SEQ ID NO: 239 - protein, 108 aa, synthetic construct
  REGION 1..108: note = Synthetic
EIVLTQSPGTLSLSPGERATLSCRASQNISTAYLAWYQQKPGQAPRLLIYGTSRRATGIPDRFSGSGSGTDFTLTISRLEPEDFAVYYCQLYGTSSVTFGQGTKLEIK

SEQ ID NO: 240 - protein, 108 aa, synthetic construct
  REGION 1..108: note = Synthetic
EIVLTQSPGTLSLSPGERATLSCRASQSVSTAYLAWYQQKPGQAPRLLIYGTSRRATGIPDRFSGSGSGTDFTLTISRLEPEDFAVYYCQLYGATSVTFGQGTKLEIK

SEQ ID NO: 241 - protein, 108 aa, synthetic construct; identical to SEQ ID NO: 234
  REGION 1..108: note = Synthetic

SEQ ID NO: 242 - protein, 108 aa, synthetic construct
  REGION 1..108: note = Synthetic
EIVLTQSPGTLSLSPGERATLSCRASQGVSTAYLAWYQQKPGQAPRLLIYGTSRRATGIPDRFSGSGSGTDFTLTISRLEPEDFAVYYCQLYGATSVTFGQGTKLEIK

SEQ ID NO: 243 - protein, 108 aa, synthetic construct
  REGION 1..108: note = Synthetic
EIVLPQSPGTLSLSPGERATLSCRASQGVSSAYLAWYQQKPGQAPRLLIYGTSRRATGIPDRFSGSGSGTDFTLTISRLEPEDFAVYYCQLYGSTSVTFGQGTKLEIK

SEQ ID NO: 244 - protein, 118 aa, synthetic construct
  REGION 1..118: note = Synthetic
EVQLVESGGGLVQPKGSLKLSCAASGFTFNTYGMHWVRQAPGKGLEWVAHIRSKSSNYATYYADSVKDRFTISRDDSQSMLYLQMNNLKTEDTAMYYCVRWFRAMDYWGQGTSVTVSS

SEQ ID NO: 245 - protein, 120 aa, synthetic construct
  REGION 1..120: note = Synthetic
EVQLIESGGGLVQPKGSLKLSCAASGFTFNMYAMDWVRQAPGKGLEWVARIRSKGSNFETNYADSVKDRFTISRDDSQSMVYLQMINLKTEDTAMYYCVRHRGGAWFAYWGQGTLVSVSA

SEQ ID NO: 246 - protein, 121 aa, synthetic construct
  REGION 1..121: note = Synthetic
QVQLVETGGGLVRPGNSLKLSCVTSGFTFSNYRMHWLRQPPGKRLEWIAVITVKSNNYGANYAESVKGRFAISRDDSKSSVYLEMNRLREEDTATYFCSRERAYGNPFDYWGQGTTLTVSS

SEQ ID NO: 247 - protein, 118 aa, synthetic construct
  REGION 1..118: note = Synthetic
EVQLVESGGGLVQPKGSLKLSCAASGFTFNMYAMNWVRQAPGQGLEWVARIRSKSNNYATYYADSVKDRFIISRDDSESMVYLQMSNLRAADTAMYYCVRHLRAMDYWGQGTSVTVSS

SEQ ID NO: 248 - protein, 118 aa, synthetic construct
  REGION 1..118: note = Synthetic
EVQLVESGGGLVQPKGSLKLSCAASGFSFNMYAMNWVRQAPGKGLEWVARIRTKSNHYSTYYADSVKDRFTISRDDSASMFYLQMNNLKTEDTAMYFCVRHLRAMDYWGQGTSVTVSS

SEQ ID NO: 249 - protein, 120 aa, synthetic construct
  REGION 1..120: note = Synthetic
EVQLIESGGGLVQPKGSLKLSCAASGFTFNMYAMDWVRQAPGKGLEWVARIRSKGSNFETNYADSVKDRFTISRDDSQSMVYLQMNNLKTEDTAMYYCVRHRGGAWFAYWGQGTLVTVSA

SEQ ID NO: 250 - protein, 118 aa, synthetic construct
  REGION 1..118: note = Synthetic
EVQLVESGGGLVRPKGSLKLSCAASGFSFNTYAMNWVRQAPGKGLEWIVWIRSKSHNYATYYADSVKDRFTISRDDSESMLYLQMNNLKTEDTAMYYCVRHLRAMDYWGQGTSVTVSS

SEQ ID NO: 251 - protein, 118 aa, synthetic construct
  REGION 1..118: note = Synthetic
EVRLVESGGGLVQPKGSLKLSCEASGFSFNMYAMNWVRQAPGKGLEWITHIRSKSNNYATYYADSVKDRFIISRDDSESMVYLQMNNLKTEDTAMYYCVRLLRALDYWGQGTSVTVSS

SEQ ID NO: 252 - protein, 118 aa, synthetic construct
  REGION 1..118: note = Synthetic
EVQLVESGGGLVQPKGSLKLSCAASGFTFNMYGMHWVRQAPGKGLEWVAHIRSKSSNYATYYADSVKDRLTISRDDSQSMLYLQMNNLKTEDTAMYYCVRWFRAMDYWGQGTSVTVSS

SEQ ID NO: 253 - protein, 118 aa, synthetic construct
  REGION 1..118: note = Synthetic
EVQLVESGGGLVKPGGSLRLSCAASGFTFSMYGMHWVRQAPGKGLEWVGHIRSKSSNYATYYADSVKDRFTISRDDSKNTLYLQMNSLKTEDTAVYYCTTWFRAMDYWGQGTLVTVSS

SEQ ID NO: 254 - protein, 118 aa, synthetic construct
  REGION 1..118: note = Synthetic
EVQLVESGGGLVQPGGSLRLSCAASGFTFSMYGMHWVRQAPGKGLEWVSHIRSKSSNYATYYADSVKDRFTISRDNAKNSLYLQMNSLRAEDTAVYYCARWFRAMDYWGQGTLVTVSS

SEQ ID NO: 255 - protein, 118 aa, synthetic construct
  REGION 1..118: note = Synthetic
EVQLVESGGGLVQPGGSLKLSCAASGFTFSMYGMHWVRQASGKGLEWVGHIRSKSSNYATYYADSVKDRFTISRDDSKNTAYLQMNSLKTEDTAVYYCTRWFRAMDYWGQGTLVTVSS

SEQ ID NO: 256 - protein, 118 aa, synthetic construct
  REGION 1..118: note = Synthetic
EVQLVESGGGLVQPGGSLRLSCAASGFTFSMYGMHWVRQAPGKGLEWVGHIRSKSSNYATYYADSVKDRFTISRDDSKNSLYLQMNSLKTEDTAVYYCARWFRAMDYWGQGTLVTVSS

SEQ ID NO: 257 - protein, 118 aa, synthetic construct; identical to SEQ ID NO: 254
  REGION 1..118: note = Synthetic

SEQ ID NO: 258 - protein, 118 aa, synthetic construct
  REGION 1..118: note = Synthetic
EVQLVESGGGLEQPGGSLRLSCAGSGFTFRMYGMHWVRQAPGKGLEWVSHIRSKSSNYATYYADSVKDRFTISRDNSKNTLYLQMNSLRAEDTAVYYCAKWFRAMDYWGQGTTVTVSS

SEQ ID NO: 259 - protein, 118 aa, synthetic construct
  REGION 1..118: note = Synthetic
EVQLVESGGGLVQPGGSLKLSCAASGFTFSMYGMHWVRQASGKGLEWVGHIRSKSSNYATYYADSVKDRFTISRDDSKNTAYLQMNSLKTEDTAVYYCTRWFRAMDYWGQGTTVTVSS

SEQ ID NO: 260 - protein, 107 aa, synthetic construct
  REGION 1..107: note = Synthetic
DIVMTQSHKFMSTSVGDRVSITCKASQDVSTAVAWYQEKPGQSPKLLIYWASTRHTGVPDRFTGSGSGTDYTLTISSVQAEDLALYYCQQHYSTPLTFGAGTKLELK

SEQ ID NO: 261 - protein, 111 aa, synthetic construct
  REGION 1..111: note = Synthetic
DIVVTQSPASLAVSLGQRATISCRASKSVSTSGYSYMHWYQQKPGQPPKLLIYLASNLESGVPARFSGSGSGTDFTLNIHPVEEEDVAIYYCQHSRELPLTFGAGTKLELK

SEQ ID NO: 262 - protein, 107 aa, synthetic construct
  REGION 1..107: note = Synthetic
DIQMTQSPSSLSASLGERVSLTCRASQEISGYLSWLQQKPDGTIKRLIYAASTLDSGVPKRFSGSRSGSDYSLTISSLESEDFADYYCLQYGSYPYTFGGGTKLEIK

SEQ ID NO: 263 - protein, 111 aa, synthetic construct
  REGION 1..111: note = Synthetic
DIVLTQSPASLTVSLGQRATISCRASKSVSTSGYSYMHWYQQKPGQPPKLLIYLASNLESGVPARFSGSGSGTDFTLNIHPVEEEDAATYYCQHSRELPITFGSGTKLEIK

SEQ ID NO: 264 - protein, 111 aa, synthetic construct
  REGION 1..111: note = Synthetic
DIVLTQSPASLVVSLGQRATISCRASQSVSTSGYSYMHWYQQKPGQPPKLLIYLASNVQSGVPARFSGSGSGTDFTLNIHPVEEEDVATYYCHHNRDLPFTFGSGTKLEIK

SEQ ID NO: 265 - protein, 111 aa, synthetic construct; identical to SEQ ID NO: 261
  REGION 1..111: note = Synthetic

SEQ ID NO: 266 - protein, 111 aa, synthetic construct
  REGION 1..111: note = Synthetic
DIVLTQSPASLAVSLGQRATISCRASKSVSASGYSYMHWYQQKPGQPPKLLIYLASNLQSGVPARFSGSGSGTDFTLNIHPVEEEDAATYYCQHSRELPPTFGGGTKLEIK

SEQ ID NO: 267 - protein, 111 aa, synthetic construct
  REGION 1..111: note = Synthetic
DIVLTQSPASLAVFLGQRATISCRASKSVSTSGYSYMHWYQQKAGQPPKLLIYLASNLESGVPARFSGSGSGTDFTLNIHPVEEEDAATYYCHHSRELPITFGSGTKLEMK

SEQ ID NO: 268 - protein, 107 aa, synthetic construct; identical to SEQ ID NO: 260
  REGION 1..107: note = Synthetic

SEQ ID NO: 269 - protein, 107 aa, synthetic construct
  REGION 1..107: note = Synthetic
EIVLTQSPGTLSLSPGERATLSCKASQDVSTAVAWYQQKPGQAPRLLIYWASTRHTGIPDRFSGSGSGTDFTLTISRLEPEDFAVYYCQQHYSTPLTFGQGTKVEIK

SEQ ID NO: 270 - protein, 107 aa, synthetic construct
  REGION 1..107: note = Synthetic
DIVMTQSPLSLPVTPGEPASISCKASQDVSTAVAWYLQKSGQSPQLLIYWASTRHTGVPDRFSGSGSGTDFTLKISRVEAEDVGFYYCQQHYSTPLTFGQGTKLEIK

SEQ ID NO: 271 - protein, 107 aa, synthetic construct
  REGION 1..107: note = Synthetic
DIVMTQSPDSLAVSLGERATINCKASQDVSTAVAWYQQKPGQPPKLLIYWASTRHTGVPDRFSGSGSGTDFTLTISSLQAEDVAVYYCQQHYSTPLTFGGGTKVEIK

SEQ ID NO: 272 - protein, 107 aa, synthetic construct
  REGION 1..107: note = Synthetic
DIQMTQSPSSLSASVGDRVTITCKASQDVSTAVAWYQQKPGKVPKLLIYWASTRHTGVPSRFSGSGSGTDFTLTISSLQPEDVATYYCQQHYSTPLTFGGGTKVEIK

SEQ ID NO: 273 - protein, 107 aa, synthetic construct
  REGION 1..107: note = Synthetic
DIQLTQSPSFLSASVGDRVTITCKASQDVSTAVAWYQQKPGKAPKLLIYWASTRHTGVPSRFSGSGSGTEFTLTISSLQPEDFATYYCQQHYSTPLTFGGGTKVEIK

SEQ ID NO: 274 - protein, 107 aa, synthetic construct
  REGION 1..107: note = Synthetic
DIQMTQSPSSLSASVGDRVTITCKASQDVSTAVAWYQQKPGKAPKLLLYWASTRHTGVPSRFSGSGSGTDYTLTISSLQPEDFATYYCQQHYSTPLTFGGGTKVEIK

SEQ ID NO: 275 - protein, 107 aa, synthetic construct
  REGION 1..107: note = Synthetic
AIRMTQSPFSLSASVGDRVTITCKASQDVSTAVAWYQQKPAKAPKLFIYWASTRHTGVPSRFSGSGSGTDYTLTISSLQPEDFATYYCQQHYSTPLTFGGGTKVEIK

(19) United States
(12) Patent Application Publication
(10) Pub. No.: US 2024/0024472 A1
(43) Pub. Date: Jan. 25, 2024

(54) Anti-Interleukin-4 Receptor (IL-4R) Antibody Formulations

(71) Applicant: Regeneron Pharmaceuticals, Inc., Tarrytown, NY (US)

(72) Inventor: Xiaolin Tang, Old Tappan, NJ (US)

(21) Appl. No.: 18/142,196

(22) Filed: May 2, 2023

Related U.S. Application Data
(60) Provisional application No. 63/337,532, filed on May 2, 2022.

(51) Int. Cl. A61K 39/395; A61K 47/10; C07K 16/28; A61K 9/08; A61K 47/42

(52) U.S. Cl. CPC A61K 39/39591; A61K 47/10; C07K 16/2866; A61K 9/08; A61K 47/42

(57) ABSTRACT

Pharmaceutical formulations comprising an antibody that specifically binds to human interleukin-4 receptor alpha (hIL-4Rα) are provided. The formulations may contain, in addition to an anti-IL-4Rα antibody, one or more buffers, at least one amino acid, at least one sugar, and a surfactant comprising a polyethylene glycol or a poloxamer. In one aspect, the pharmaceutical formulations do not have appreciable subvisible particle formation in the presence of lipase, and exhibit a substantial degree of antibody stability during storage and after being subjected to thermal and other physical stresses.

### CROSS-REFERENCE TO RELATED APPLICATIONS

This application claims the benefit under 35 USC § 119(e) of U.S. Provisional Application No. 63/337,532, filed May 2, 2022, the contents of which are incorporated by reference herein.

### REFERENCE TO A SEQUENCE LISTING

This application incorporates by reference a computer readable Sequence Listing in ST.26 XML format, titled 11200US01_Sequence, created on May 2, 2023 and containing 369,229 bytes.

### FIELD OF THE INVENTION

The present invention relates to the field of therapeutic antibody formulations. More specifically, the present invention relates to the field of pharmaceutical formulations comprising a human antibody that specifically binds to human interleukin-4 receptor alpha.

### BACKGROUND

Interleukin-4 (IL-4) and interleukin-13 (IL-13) are key cytokines in driving allergic and T helper cell type 2 (Th2) polarized inflammatory processes. IL-4 and IL-13 signaling is mediated through heterodimeric receptor complexes, in which IL-4 receptor alpha (IL-4Rα) is a shared receptor subunit for both IL-4 and IL-13 signaling. Thus, IL-4Rα is an attractive therapeutic target because it provides a single target for blocking both IL-4 and IL-13 signaling. Antibodies to hIL-4Rα are described in, for example, U.S. Pat. Nos. 5,717,072, 7,186,809 and 7,605,237.

Therapeutic macromolecules (e.g., antibodies) must be formulated in a manner that not only makes the molecules suitable for administration to patients, but also maintains their stability during storage. For example, therapeutic antibodies in liquid solution are prone to degradation, aggregation and/or undesired chemical modifications unless the solution is formulated properly. The stability of an antibody in liquid formulation depends not only on the kinds of excipients used in the formulation, but also on the amounts and proportions of the excipients relative to one another. Therapeutic formulations also may be subject to the formation of particulate matter over time during storage. Particles may be visible or subvisible; subvisible particles typically are under 150 microns or 100 microns in diameter. Formulations having high protein concentrations, e.g., concentrations of 30 mg/mL or higher, are more prone to aggregation and subvisible particle formation.

Polysorbate 20 and polysorbate 80 are commonly used in formulating biologic macromolecules such as antibodies. However, it has been reported that polysorbates, including polysorbate 20 and polysorbate 80, can degrade in the presence of lipase, which over time results in the formation of subvisible particles in a formulation. Without intending to be bound by theory, it is believed that a putative phospholipase B-like 2 (PLBL2), which is highly conserved in hamster, rat, mice, human and bovine, copurifies with some classes of proteins under certain processes. One solution to this problem is to employ a protein purification process that removes or decreases the activity of the lipase. See, e.g., U.S. Pat. No. 10,342,876. However, there remains a need for alternatives for formulating antibodies, such as anti-hIL-4Rα antibodies, in such a way that avoids or minimizes subvisible particle formation, while still maintaining sufficient stability and suitability for administration to patients.

### SUMMARY

Provided herein are stable liquid pharmaceutical formulations comprising an anti-IL-4Rα antibody and one or more excipients, as well as delivery devices, kits and unit dosage forms comprising such formulations.

In one aspect, the present disclosure provides a stable liquid pharmaceutical formulation comprising: (i) a human antibody, wherein the antibody specifically binds to human interleukin-4 receptor alpha (hIL-4Rα); (ii) one or more buffers; (iii) a thermal stabilizer; (iv) a viscosity reducer; and (v) a surfactant comprising a polyethylene glycol or a poloxamer at a concentration of less than 0.2% w/v, or a polyethylene glycol at a concentration of less than 1% w/v, wherein the formulation has a pH of from 5.7 to 6.1.

In one aspect, the present disclosure provides a stable liquid pharmaceutical formulation comprising: (i) a human antibody, wherein the antibody specifically binds to human interleukin-4 receptor alpha (hIL-4Rα) and comprises a heavy chain variable region (HCVR) comprising one or more sequences disclosed in Table 1; (ii) acetate at a concentration of from 10 mM to 15 mM; (iii) histidine at a concentration of from 15 mM to 25 mM; (iv) sucrose at a concentration of from 2.5% w/v to 7.5% w/v; (v) arginine at a concentration of from 20 mM to 80 mM; and (vi) a surfactant comprising a polyethylene glycol or a poloxamer at a concentration of less than 0.2% w/v, or a polyethylene glycol at a concentration of less than 1% w/v, wherein the formulation has a pH of from 5.7 to 6.1.

In some embodiments, the antibody comprises a heavy chain variable region (HCVR) comprising complementarity determining regions HCDR1, HCDR2, and HCDR3 comprising the amino acid sequences of SEQ ID NOs: 3, 4, and 5, respectively, and a light chain variable region (LCVR) comprising complementarity determining regions LCDR1, LCDR2, and LCDR3 comprising the amino acid sequences of SEQ ID NOs: 6, 7, and 8, respectively. In some embodiments, the antibody comprises a HCVR comprising the amino acid sequence of SEQ ID NO: 1, and a LCVR comprising the amino acid sequence of SEQ ID NO: 2.

In some embodiments, the antibody has a human IgG heavy chain constant region. In some cases, the heavy chain constant region is of isotype IgG1. In some cases, the heavy chain constant region is of isotype IgG4.

In some embodiments, the antibody comprises a heavy chain comprising the amino acid sequence of SEQ ID NO: 9, and a light chain comprising the amino acid sequence of SEQ ID NO: 10.

In some embodiments, the surfactant is a polyethylene glycol. In some cases, the polyethylene glycol is PEG3350. In some embodiments, the surfactant is present at a concentration of from 0.0075% w/v to less than 1% w/v, from 0.0075% w/v to 0.75% w/v, from 0.01% w/v to 0.5% w/v, or from 0.01% w/v to 0.25% w/v, In some embodiments, the surfactant is present at a concentration of from 0.01% w/v to 0.19% w/v, e.g., from 0.01% w/v to 0.15% w/v or from 0.05% w/v to 0.15% w/v. In some embodiments, the surfactant is present at a concentration of no more than 0.15% w/v.

In some embodiments, the surfactant is a poloxamer. In some cases, the poloxamer is poloxamer 188. In some embodiments, the surfactant is present at a concentration of from 0.0075% w/v to 0.19% w/v, or from 0.01% w/v to 0.19% w/v, e.g., from 0.01% w/v to 0.15% w/v or from 0.05% w/v to 0.15% w/v. In some embodiments, the surfactant is present at a concentration of no more than 0.15% w/v.

In some embodiments, the buffer comprises an acetate buffer and/or a histidine buffer. In some embodiments, the buffer comprises acetate at a concentration of from 10 mM to 15 mM and/or histidine at a concentration of from 15 mM to 25 mM. In some embodiments, the acetate is present at a concentation of 12.5 mM±1.25 mM. In some embodiments, the histidine is present at a concentration of 20 mM±2 mM. In some embodiments, the thermal stabilizer is sucrose at a concentration of 5% w/v±0.5% w/v. In some embodiments, the viscosity reducer is arginine at a concentration of 25 mM±2.5 mM. In some embodiments, the arginine is present at a concentration of 50 mM±2.5 mM. In some embodiments, the arginine is present at a concentration of 75 mM±2.5 mM.

In some embodiments, the antibody is present at a concentration of up to 200 mg/mL. In some embodiments, the antibody is present at a concentration of from 15 mg/mL to 200 mg/mL. In some embodiments, the antibody is present at a concentration of from 100 mg/mL to 200 mg/mL. In some cases, the antibody is present at a concentration of 150 mg/mL±15 mg/mL. In some cases, the antibody is present at a concentration of 175 mg/mL±15 mg/mL.

In one aspect, the present disclosure provides a stable liquid pharmaceutical formulation comprising: (i) a human antibody at a concentration of 150 mg/mL±10 mg/mL, wherein the antibody specifically binds to human interleukin-4 receptor alpha (hIL-4Rα) and comprises a heavy chain variable region (HCVR) comprising the amino acid sequence of SEQ ID NO: 1, and a light chain variable region (LCVR) comprising the amino acid sequence of SEQ ID NO: 2; (ii) acetate at a concentation of 12.5 mM±1.25 mM; (iii) histidine at a concentration of 20 mM±2 mM; (iv) sucrose at a concentration of 5% w/v±0.5% w/v; (v) arginine at a concentration of 25 mM±2.5 mM; and (vi) a surfactant comprising PEG3350 or poloxamer 188 at a concentration of from 0.01% w/v to 0.19% w/v, wherein the formulation has a pH of 5.9±0.3 or a pH of 5.9±0.2. In some cases, the antibody comprises a heavy chain comprising the amino acid sequence of SEQ ID NO: 9, and a light chain comprising the amino acid sequence of SEQ ID NO: 10.

In one aspect, the present disclosure provides a stable liquid pharmaceutical formulation comprising: (i) a human antibody at a concentration of 175 mg/mL±10 mg/mL, wherein the antibody specifically binds to human interleukin-4 receptor alpha (hIL-4Rα) and comprises a heavy chain variable region (HCVR) comprising the amino acid sequence of SEQ ID NO: 1, and a light chain variable region (LCVR) comprising the amino acid sequence of SEQ ID NO: 2; (ii) acetate at a concentation of 12.5 mM±1.25 mM; (iii) histidine at a concentration of 20 mM±2 mM; (iv) sucrose at a concentration of 5% w/v±0.5% w/v; (v) arginine at a concentration of 25 mM±2.5 mM; and (vi) a surfactant comprising PEG3350 or poloxamer 188 at a concentration of from 0.01% w/v to 0.19% w/v, wherein the formulation has a pH of 5.9±0.3 or a pH of 5.9±0.2. In some cases, the antibody comprises a heavy chain comprising the amino acid sequence of SEQ ID NO: 9, and a light chain comprising the amino acid sequence of SEQ ID NO: 10.

In some embodiments, the formulation comprises a phospholipase. In some embodiments, the phospholipase protein is phospholipase B-like 2 protein.

In some embodiments, the stable liquid pharmaceutical formulation as discussed herein comprises no more than 4% high molecular weight (HMW) species following agitation for 24 hours at 25° C., as determined by SE-UPLC. In some embodiments, the formulation comprises no more than 4% high molecular weight (HMW) species following agitation for 24 hours at 25° C. and storage at (i) 5° C. for 6, 12, or 18 months, or (ii) 25° C. for 6 months, as determined by SE-UPLC.

In some embodiments, the formulation comprises no more than 3% high molecular weight (HMW) species following agitation by vortexing for up to 120 minutes at room temperature, as determined by size exclusion-ultra performance liquid chromatography (SE-UPLC). In some cases, the formulation comprises no more than 2.5% high molecular weight (HMW) species following agitation by vortexing for up to 120 minutes at room temperature, as determined by SE-UPLC.

In some embodiments, the formulation comprises no more than 8% high molecular weight (HMVV) species after 30 days of storage at 45° C., as determined by SE-UPLC. In some cases, the formulation comprises no more than 9% high molecular weight (HMW) species after 40 days of storage at 45° C., as determined by SE-UPLC. In some cases, the formulation comprises no more than 11% high molecular weight (HMW) species after 50 days of storage at 45° C., as determined by SE-UPLC.

In some embodiments, the formulation comprises no more than 1000, no more than 500, no more than 300, or no more than 150 particles having a diameter of ≥10 μm in a volume of 2.25 mL after storage for 6 months at 5° C., as determined by microscopy, and wherein the formulation contains a phospholipase protein.

In some embodiments, the formulation comprises no more than 1000, no more than 500, no more than 300, or no more than 150 particles having a diameter of ≥10 μm in a volume of 2.25 mL after storage for 36 months at 5° C., as determined by microscopy, and wherein the formulation contains a phospholipase protein.

In some embodiments, the formulation comprises no more than 500, no more than 300, no more than 250, or no more than 50 particles having a diameter of ≥25 μm in a volume of 2.25 mL after storage for 6 months at 5° C., as determined by microscopy, and wherein the formulation contains a phospholipase protein.

In some embodiments, the formulation comprises no more than 500, no more than 300, no more than 250, or no more than 50 particles having a diameter of ≥25 μm in a volume of 2.25 mL after storage for 6 months at 5° C., as determined by microscopy, and wherein the formulation contains a phospholipase protein.

In some embodiments, the formulation does not include an esterase inhibitor.

In some embodiments, the stable liquid pharmaceutical formulation is contained in a glass vial.

In some embodiments, the stable liquid pharmaceutical formulation is contained in a syringe. In some cases, the syringe comprises a fluorocarbon-coated plunger. In some cases, the syringe is a low tungsten syringe. In some cases, the syringe is a prefilled syringe. In some cases, the syringe is a prefilled staked needle syringe.

In some embodiments, the stable liquid pharmaceutical formulation is contained in a large volume device or bolus injector.

In one aspect, the present disclosure provides a pen or autoinjector delivery device containing a stable liquid pharmaceutical formulation as discussed above or herein. In some cases, the deliver device is a disposable pen delivery device. In some cases, the delivery device is a reusable pen delivery device.

In one aspect, the present disclosure provides a container containing a stable liquid pharmaceutical formulation as discussed above or herein.

In one aspect, the present disclosure provides a kit comprising (i) a container containing a stable liquid pharmaceutical formulation as discussed above or herein, and (ii) labeling for use of the pharmaceutical formulation. In some cases, the labeling recites subcutaneous administration of the pharmaceutical formulation. In some cases, the labeling recites intravenous administration of the pharmaceutical formulation.

In one aspect, the present disclosure provides a unit dosage form comprising a stable liquid pharmaceutical formulation as discussed above or herein, wherein the antibody is present in an amount of from 1 mg to 500 mg. In some cases, the antibody is present in an amount of about 100 mg. In some cases, the antibody is present in an amount of about 200 mg. In some cases, the antibody is present in an amount of about 300 mg. In some embodiments, the formulation of the unit dosage form is contained in a syringe. In some cases, the syringe is a prefilled syringe. In some embodiments, the formulation of the unit dosage form is contained in a prefilled pen. In some embodiments, the formulation of the unit dosage form is contained in an autoinjector.

In one aspect, the present disclosure provides a safety system delivery device containing a stable liquid pharmaceutical formulation as discussed above or herein. In some cases, the safety system delivery device includes a safety sleeve configured to extend by manual operation. In some cases, the safety system delivery device includes a safety sleeve configured to automatically extend following injection of the stable liquid pharmaceutical formulation.

In various embodiments, any of the features or components of embodiments discussed above or herein may be combined, and such combinations are encompassed within the scope of the present disclosure. Any specific value discussed above or herein may be combined with another related value discussed above or herein to recite a range with the values representing the upper and lower ends of the range, and such ranges are encompassed within the scope of the present disclosure. Each of the values discussed above or herein may be expressed with a variation of 1%, 5%, 10% or 20%. For example, a concentration of 10 mM may be expressed as 10 mM±0.1 mM (1% variation), 10 mM±0.5 mM (5% variation), 10 mM±1 mM (10% variation) or 10 mM±2 mM (20% variation).

Other embodiments will become apparent from a review of the detailed description.

### BRIEF DESCRIPTION OF THE DRAWINGS

FIG. 1 shows the number of particles ≥10 μm (upper panel) and ≥25 μm (lower panel) identified by microscopy in a formulation of 150 mg/mL of an anti-IL-4R antibody containing a lipase and either PEG3350 or poloxamer 188 (at concentrations of 0.02%, 0.04%, or 0.1% w/v) and stored at 5° C. for up to 36 months.

FIGS. 2A and 2B show the impact of PEG3350 concentrations (FIG. 2A) and poloxamer 188 concentrations (FIG. 2B) on the stability (as a percentage of high molecular weight (HMW species measured by SE-UPLC) of a formulation of 150 mg/mL of an anti-IL-4R antibody against agitation stress at room temperature for a period of from 30-120 minutes.

FIG. 3 shows the impact of polysorbate 20, polysorbate 80, PEG3350, and poloxamer 188 (at varying concentrations) on the stability (as a percentage of high molecular weight (HMW species measured by SE-UPLC) of a formulation of 150 mg/mL of an anti-IL-4R antibody against thermal stress (45° C.) for a period of up to 56 days.

### DETAILED DESCRIPTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. As used herein, the term “about,” when used in reference to a particular recited numerical value or range of values, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the present invention, exemplary methods and materials are now described. All patents, applications and non-patent publications mentioned in this specification are incorporated herein by reference in their entireties.

### Pharmaceutical Formulations

As used herein, the expression “pharmaceutical formulation” means a combination of at least one active ingredient (e.g., an anti-IL-4Rα antibody, etc. which is capable of exerting a biological effect in a human or non-human animal), and at least one inactive ingredient which, when combined with the active ingredient and/or one or more additional inactive ingredients, is suitable for therapeutic administration to a human or non-human animal. The term “formulation,” as used herein, means “pharmaceutical formulation” unless specifically indicated otherwise. The present disclosure provides pharmaceutical formulations comprising at least one therapeutic polypeptide. According to certain embodiments of the present disclosure, the therapeutic polypeptide is an antibody that binds specifically to human interleukin-4 receptor alpha (hIL-4Rα) or an antigen-binding fragment thereof. More specifically, the present disclosure includes pharmaceutical formulations that comprise: (i) a human antibody that specifically binds to hIL-4Rα; (ii) one or more buffers; (iii) a thermal stabilizer; (iv) a surfactant (e.g., organic cosolvent); and (v) a viscosity modifier. Additional components may be included in the formulations of the present disclosure if such components do not significantly interfere with the viscosity and stability of the formulation. Specific exemplary components and formulations included within the present disclosure are described in detail below.

The pharmaceutical formulations of the present disclosure may, in certain embodiments, be fluid formulations. As used herein, the expression “fluid formulation” means a mixture of at least two components that exists predominantly in the fluid state at about 2° C. to about 45° C. Fluid formulations include, inter alia, liquid formulations. Fluid formulations may be of low, moderate or high viscosity depending on their particular constituents.

### Antibodies That Specifically Bind Human IL-4 Receptor Alpha

The pharmaceutical formulations of the present disclosure may comprise a human antibody, or an antigen-binding fragment thereof, that binds specifically to human interleukin-4 receptor alpha (IL-4Rα). References to IL-4R antibodies, herein, also refer to antibodies that bind hIL-4Rα.

The term “antibody,” as used herein, is generally intended to refer to immunoglobulin molecules comprising four polypeptide chains, two heavy (H) chains and two light (L) chains inter-connected by disulfide bonds, as well as multimers thereof (e.g., IgM); however, immunoglobulin molecules consisting of only heavy chains (i.e., lacking light chains) are also encompassed within the definition of the term “antibody.” Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementary determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

In certain embodiments of the disclosure, the anti-IL-4R antibodies of the disclosure are human antibodies. The term “human antibody,” as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences. In various embodiments, the anti-IL-4R antibody is a human IgG antibody. In various embodiments, the anti-IL-4R antibody is a human antibody of isotype IgG1, IgG2, IgG3 or IgG4, or mixed isotype. In some embodiments, the anti-IL-4R antibody is a human IgG1 antibody. In some embodiments, the anti-IL-4R antibody is a human IgG4 antibody. In any of the embodiments discussed above or herein, the anti-IL-4R antibody may comprise a human kappa light chain. In any of the embodiments discussed above or herein, the anti-IL-4R antibody may comprise a human lambda light chain.

The antibodies of the disclosure may, in some embodiments, be recombinant human antibodies. The term “recombinant human antibody,” as used herein, is intended to include all human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The terms “antigen-binding portion” or “antigen-binding fragment” of an antibody (or simply “antibody portion” or “antibody fragment”), as used herein, refer to one or more fragments of an antibody that retain the ability to specifically bind to hIL-4Rα.

An “isolated antibody,” as used herein, is intended to refer to an antibody that is substantially free of other antibodies having different antigenic specificities (e.g., an isolated antibody that specifically binds hIL-4Rα is substantially free of antibodies that specifically bind antigens other than hIL-4Rα).

The term “specifically binds,” or the like, means that an antibody or antigen-binding fragment thereof forms a complex with an antigen that is relatively stable under physiologic conditions. Specific binding can be characterized by a dissociation constant of at least about 1×10⁻⁶ M or greater. Methods for determining whether two molecules specifically bind are well known in the art and include, for example, equilibrium dialysis, surface plasmon resonance, and the like. An isolated antibody that specifically binds hIL-4Rα may, however, have cross-reactivity to other antigens, such as IL-4Rα molecules from other species (orthologs). In the context of the present disclosure, multispecific (e.g., bispecific) antibodies that bind to hIL-4Rα as well as one or more additional antigens are deemed to “specifically bind” hIL-4Rα. Moreover, an isolated antibody may be substantially free of other cellular material and/or chemicals. However, in some instances, the isolated anibody may be copurified with a phospholipase expressed by a mammalian cell line (e.g., CHO cells) from which the anti-IL-4R antibody is produced.

According to certain embodiments of the present disclosure, the anti-hIL-4R antibody, or antigen-binding fragment thereof, comprises heavy chain complementarity determining regions HCDR1-HCDR2-HCDR3, respectively, comprising the amino acid sequences of SEQ ID NOs: 3-4-5. According to certain embodiments of the present disclosure, the anti-hIL-4R antibody, or antigen-binding fragment thereof, comprises light chain complementarity determining regions LCDR1-LCDR2-LCDR3, respectively, comprising the amino acid sequences of SEQ ID NOs: 6-7-8. In certain embodiments, the anti-hIL-4R antibody, or antigen-binding fragment thereof, comprises the CDRs HCDR1-HCDR2-HCDR3-LCDR1-LCDR2-LCDR3, respectively, comprising the amino acid sequences of SEQ ID NOs:3-4-5-6-7-8. In some embodiments, the IL-4R antagonist is an anti-IL-4Rα antibody or antigen-binding fragment thereof that comprises three HCDRs (HCDR1, HCDR2 and HCDR3) and three LCDRs (LCDR1, LCDR2 and LCDR3), wherein the HCDR1 comprises or consists of the amino acid sequence GFTFRDYA (SEQ ID NO:3), the HCDR2 comprises or consists of the amino acid sequence ISGSGGNT (SEQ ID NO:4), the HCDR3 comprises or consists of the amino acid sequence AKDRLSITIRPRYYGLDV (SEQ ID NO:5), the LCDR1 comprises or consists of the amino acid sequence QSLLYSIGYNY (SEQ ID NO:6), the LCDR2 comprises or consists of the amino acid sequence LGS (SEQ ID NO:7), and the LCDR3 comprises or consists of the amino acid sequence MQALQTPYT (SEQ ID NO:8).

In certain embodiments, the anti-hIL-4R antibody, or antigen-binding fragment thereof, comprises heavy chain complementarity determining regions HCDR1-HCDR2-HCDR3, respectively, comprising the amino acid sequences of SEQ ID NOs: 3-4-5 and has a heavy chain variable region (HCVR) having at least 90% sequence identity (e.g., at least 91%, 92%, 93%, 94%, 95%, 96%, 97%, 98%, or 99% sequence identity) to the amino acid sequence of SEQ ID NO: 1. In certain embodiments, the anti-hIL-4R antibody, or antigen-binding fragment thereof, comprises light chain complementarity determining regions LCDR1-LCDR2-LCDR3, respectively, comprising the amino acid sequences of SEQ ID NOs: 6-7-8 and has a light chain variable region (LCVR) having at least 90% sequence identity (e.g., at least 91%, 92%, 93%, 94%, 95%, 96%, 97%, 98%, or 99% sequence identity) to the amino acid sequence of SEQ ID NO: 2. In certain embodiments, the anti-hIL-4R antibody, or antigen-binding fragment thereof, comprises: heavy chain complementarity determining regions HCDR1-HCDR2-HCDR3, respectively, comprising the amino acid sequences of SEQ ID NOs: 3-4-5 and has a heavy chain variable region (HCVR) having at least 90% sequence identity (e.g., at least 91%, 92%, 93%, 94%, 95%, 96%, 97%, 98%, or 99% sequence identity) to the amino acid sequence of SEQ ID NO: 1; and light chain complementarity determining regions LCDR1-LCDR2-LCDR3, respectively, comprising the amino acid sequences of SEQ ID NOs: 6-7-8 and has a light chain variable region (LCVR) having at least 90% sequence identity (e.g., at least 91%, 92%, 93%, 94%, 95%, 96%, 97%, 98%, or 99% sequence identity) to the amino acid sequence of SEQ ID NO: 2.

In certain embodiments, the anti-hIL-4R antibody, or antigen-binding fragment thereof, comprises a heavy chain variable region (HCVR) comprising the amino acid sequence of SEQ ID NO: 1. In certain embodiments, the anti-hIL-4R antibody, or antigen-binding fragment thereof, comprises a light chain variable region (LCVR) comprising the amino acid sequence of SEQ ID NO: 2. In certain embodiments, the anti-hIL-4R antibody, or antigen-binding fragment thereof, comprises a HCVR/LCVR amino acid sequence pair comprising the amino acid sequences of SEQ ID NOs: 1/2. In some embodiments, the anti-IL-4R antibody comprises a HCVR/LCVR comprising the amino acid sequences of SEQ ID NOs: 1/2, respectively, and a human IgG1 heavy chain constant region. In some embodiments, the anti-IL-4R antibody comprises a HCVR/LCVR comprising the amino acid sequences of SEQ ID NOs: 1/2, respectively, and a human IgG4 heavy chain constant region. In some embodiments, the anti-IL-4R antibody comprises a HCVR/LCVR comprising the amino acid sequences of SEQ ID NOs: 1/2, respectively, and a human IgG heavy chain constant region. In some embodiments, the anti-IL-4R antibody comprises a HCVR/LCVR comprising the amino acid sequences of SEQ ID NOs: 1/2, respectively, and a human IgG1 or IgG4 heavy chain constant region. In some embodiments, the anti-IL-4R antibody comprises a heavy chain comprising the amino acid sequence of SEQ ID NO: 9 and a light chain comprising the amino acid sequence of SEQ ID NO: 10. In some embodiments, the anti-IL-4R antibody is dupilumab.

Other anti-IL-4R antibodies that can be used in the context of the methods of the present disclosure include, e.g., the antibody referred to and known in the art as AMG317 (Corren et al., 2010, Am J Respir Crit Care Med., 181(8):788-796), or MEDI 9314, or any of the anti-IL-4Rα antibodies as set forth in U.S. Pat. Nos. 7,186,809, 7,605,237, 7,638,606, 8,092,804, 8,679,487, 8,877,189, 10,774,141; US Patent Application Publication No. US2021/0238294; or International Patent Publication Nos. WO2019/228405, WO2020/096381, WO 2020/135471, WO2020/135710, or WO 2020/239134, the contents of each of which are incorporated by reference herein.

In some embodiments, the anti-IL-4R antibody comprises one or more CDR, HCVR, and/or LCVR sequences set forth in Table 1 below.

The amount of antibody, or antigen-binding fragment thereof, contained within the pharmaceutical formulations of the present disclosure may vary depending on the specific properties desired of the formulations, as well as the particular circumstances and purposes for which the formulations are intended to be used. In certain embodiments, the pharmaceutical formulations may contain about 1 mg/mL to about 500 mg/mL of antibody; about 5 mg/mL to about 250 mg/mL of antibody; about 5 mg/mL to about 200 mg/mL of antibody; about 15 mg/mL to about 200 mg/mL; about 25 mg/mL to about 200 mg/mL of antibody; about 50 mg/mL to about 200 mg/mL of antibody; about 100 mg/mL to about 200 mg/mL; about 125 mg/mL to about 175 mg/mL; or about 150 mg/mL to about 200 mg/mL of antibody. For example, the formulations of the present disclosure may be liquid formulations that comprise about 1 mg/mL; about 2 mg/mL; about 5 mg/mL; about 10 mg/mL; about 15 mg/mL; about 20 mg/mL; about 25 mg/mL; about 30 mg/mL; about 35 mg/mL; about 40 mg/mL; about 45 mg/mL; about 50 mg/mL; about 55 mg/mL; about 60 mg/mL; about 65 mg/mL; about 70 mg/mL; about 75 mg/mL; about 80 mg/mL; about 85 mg/mL; about 90 mg/mL; about 95 mg/mL; about 100 mg/mL; about 105 mg/mL; about 110 mg/mL; about 115 mg/mL; about 120 mg/mL; about 125 mg/mL; about 130 mg/mL; about 135 mg/mL; about 140 mg/mL; about 145 mg/mL; about 150 mg/mL; about 155 mg/mL; about 160 mg/mL; about 165 mg/mL; about 170 mg/mL; about 175 mg/mL; about 180 mg/mL; about 185 mg/mL; about 190 mg/mL; about 195 mg/mL; or about 200 mg/mL of an antibody or an antigen-binding fragment thereof, that binds specifically to hIL-4Rα. In certain embodiments, the pharmaceutical formulations are liquid formulations that may contain 5±0.5 mg/mL to 200±20 mg/mL of antibody; 15±1.5 mg/mL to 200±20 mg/mL of antibody; 25±2.5 mg/mL to 200±20 mg/mL of antibody; 50±5 mg/mL to 200±20 mg/mL of antibody; 100±10 mg/mL to 200±20 mg/mL of antibody; 150±10 mg/mL of antibody; or 175±10 mg/mL. In some embodiments, the pharmaceutical formulations contain from 140±5 mg/mL to 160±5 mg/mL of the anti-IL-4R antibody. In some cases, the pharmaceutical formulations contain 165 mg/mL±5 mg/mL to 185 mg/mL±5 mg/mL of the anti-IL-4R antibody. In some cases, the pharmaceutical formulations contain 150 mg/mL±5 mg/mL of the anti-IL-4R antibody. In some cases, the pharmaceutical formulations contain 175 mg/mL±5 mg/mL of the anti-IL-4R antibody.

### Bioequivalents

The present disclosure encompasses antibodies having amino acid sequences that vary from those of the exemplary molecules disclosed herein but that retain the ability to bind hIL-4R. Such variant molecules may comprise one or more additions, deletions, or substitutions of amino acids when compared to parent sequence, but exhibit biological activity that is essentially equivalent to that of the antibodies discussed herein.

The present disclosure includes antigen-binding molecules that are bioequivalent to any of the exemplary antibodies set forth herein. In some embodiments, the antigen-binding molecule is a bioequivalent of dupilumab. Two antibodies are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antibodies are bioequivalent if there are no clinically meaningful differences in their safety, purity, and potency.

In one embodiment, two antibodies are bioequivalent if a patient can be switched one or more times between the reference product and the biological product without an expected increase in the risk of adverse effects, including a clinically significant change in immunogenicity, or diminished effectiveness, as compared to continued therapy without such switching.

Bioequivalence may be demonstrated by in vivo and in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antigen-binding protein.

### Formulation Excipients and pH

The pharmaceutical formulations of the present disclosure comprise one or more excipients. The term “excipient,” as used herein, means any non-therapeutic agent added to the formulation to provide a desired consistency, viscosity or stabilizing effect.

The pharmaceutical formulations of the present disclosure may also comprise a buffer or buffer system, which serves to maintain a stable pH and to help stabilize the anti-IL-4R antibody. In some embodiments, the buffer or buffer system comprises at least one buffer that has a buffering range that overlaps fully or in part the range of pH 5.5 to 6.3. In various embodiments, the pH of the formulation is 5.5, 5.6, 5.7, 5.8, 5.9, 6.0, 6.1, 6.2 or 6.3. In some embodiment, the formulations have a pH of 5.9±0.3. In some embodiment, the formulations have a pH of 5.9±0.2. In some embodiment, the formulations have a pH of 5.9±0.1. In certain embodiments, the buffer comprises a histidine buffer. In certain embodiments, the buffer comprises an acetate buffer. In certain embodiments, the buffer (e.g., histidine and/or acetate) is present at a concentration of from about 1 mM to about 40 mM, about 5 mM to about 30 mM, about 10 mM to about 15 mM; or about 15 mM to about 25 mM. In some embodiments, the buffer includes a histidine buffer at a concentration of from 15 mM to 25 mM. In some embodiments, the buffer includes a histidine buffer at a concentration of 20 mM±2 mM. In some cases, the histidine buffer is present at a concentration of 15 mM, 16 mM, 17 mM, 18 mM, 19 mM, 20 mM, 21 mM, 22 mM, 23 mM, 24 mM, or 25 mM. In some embodiments, the buffer comprises an acetate buffer at a concentration of from 10 mM to 15 mM. In some embodiments, the buffer comprises an acetate buffer at a concentation of 12.5 mM±1.25 mM. In some cases, the acetate buffer is present at a concentration of 10 mM, 10.5 mM, 11 mM, 11.5 mM, 12 mM, 12.5 mM, 13 mM, 13.5 mM, 14 mM, 14.5 mM, or 15 mM. In some embodiments, the formulations of the present disclosure include both histidine and acetate buffers at any of the concentrations discussed above. In some cases, the formulations contain a histidine buffer at a concentration of from 15 mM to 25 mM, and an acetate buffer at a concentration of from 10 mM to 15 mM. In some cases, the formulations contain a histidine buffer at a concentration of 20 mM±2 mM, and an acetate buffer at a concentration of 12.5 mM±1.25 mM.

The pharmaceutical formulations of the present disclosure may also comprise one or more carbohydrates, e.g., one or more sugars. The sugar can be a reducing sugar or a non-reducing sugar. “Reducing sugars” include, e.g., sugars with a ketone or aldehyde group and contain a reactive hemiacetal group, which allows the sugar to act as a reducing agent. Specific examples of reducing sugars include fructose, glucose, glyceraldehyde, lactose, arabinose, mannose, xylose, ribose, rhamnose, galactose and maltose. Non-reducing sugars can comprise an anomeric carbon that is an acetal and is not substantially reactive with amino acids or polypeptides to initiate a Maillard reaction. Specific examples of non-reducing sugars include sucrose, trehalose, sorbose, sucralose, melezitose and raffinose. Sugar acids include, for example, saccharic acids, gluconate and other polyhydroxy sugars and salts thereof. In some embodiments, the sugar is sucrose. In some cases, the sugar (e.g., sucrose) acts as a thermal stabilizer for the anti-IL-4R antibody.

The amount of sugar (e.g., sucrose) contained within the pharmaceutical formulations of the present disclosure will vary depending on the specific circumstances and intended purposes for which the formulations are used. In certain embodiments, the formulations may contain about 0.1% to about 20% sugar; about 0.5% to about 20% sugar; about 1% to about 20% sugar; about 2% to about 15% sugar; about 3% to about 8% sugar; or about 4% to about 6% sugar. For example, the pharmaceutical formulations of the present disclosure may comprise about 0.5%; about 1.0%; about 1.5%; about 2.0%; about 2.5%; about 3.0%; about 3.5%; about 4.0%; about 4.5%; about 5.0%; about 5.5%; about 6.0%; about 6.5%; about 7.0%; about 7.5%; about 8.0%; about 8.5%; about 9.0%; about 9.5%; about 10.0%; about 15%; or about 20% sugar (e.g., sucrose). In some embodiments, the formulations contain about 5% sugar (e.g., sucrose). In some embodiments, the formulations contain about 5%±0.5% sugar (e.g., sucrose). Each of the percentages noted above corresponds to a percent weight/volume (w/v).

The pharmaceutical formulations of the present disclosure may also comprise one or more organic cosolvents in a type and in an amount that stabilizes the anti-IL-4R antibody under conditions of rough handling or agitation, such as, e.g., orbital shaking. In some embodiments, the organic cosolvent is a surfactant. As used herein, the term “surfactant” means a substance which reduces the surface tension of a fluid in which it is dissolved and/or reduces the interfacial tension between oil and water. Surfactants can be ionic or non-ionic. Specific non-ionic surfactants that can be included in the formulations of the present disclosure include, e.g., poloxamers such as poloxamer 188 and polyethylene glycols (PEGs) such as PEG3350.

The amount of surfactant contained within the pharmaceutical formulations of the present disclosure may vary depending on the specific properties desired of the formulations, as well as the particular circumstances and purposes for which the formulations are intended to be used. In certain embodiments, the formulations may contain at least about 0.0075% surfactant. In certain embodiments, the formulations may contain at least about 0.01% surfactant. In certain embodiments, the formulations may contain about 0.0075% to about 0.5% surfactant. In certain embodiments, the formulations may contain less than 0.2% surfactant. In certain embodiments, the formulations may contain about 0.0075% to about 0.19% surfactant; about 0.01% to about 0.19% surfactant; about 0.0075% to about 0.15% surfactant; about 0.01% to about 0.15% surfactant; about 0.0075% to about 0.12% surfactant; about 0.01% to about 0.12% surfactant; about 0.0075% to about 0.11% surfactant; about 0.01% to about 0.11% surfactant; about 0.0075% to about 0.1% surfactant; about 0.01% to about 0.1% surfactant; about 0.0075% to about 0.09% surfactant; or about 0.01% to about 0.09% surfactant. For example, the formulations of the present disclosure may comprise about 0.0075%; about 0.01%; about 0.02%; about 0.03%; about 0.04%; about 0.05%; about 0.06%; about 0.07%; about 0.08%; about 0.09%; about 0.1%; about 0.11%; about 0.12%; about 0.13%; about 0.14%; about 0.15%; about 0.16%; about 0.17%; about 0.18%; or about 0.19% surfactant (e.g., poloxamer 188 or PEG3350). In some embodiments, the formulations contain less than 0.2% poloxamer (e.g., poloxamer 188). In some embodiments, the formulations contain about 0.0075% to 0.19% poloxamer 188. In some embodiments, the formulations contain about 0.01% to 0.19% poloxamer 188. In some embodiments, the formulations contain less than 1% polyethylene glycol (e.g., PEG3350). In some embodiments, the formulations contain about 0.0075% to about 0.5% PEG3350. In some embodiments, the formulations contain about 0.01% to 0.19% PEG3350. Each of the percentages noted above corresponds to a percent weight/volume (w/v).

In certain embodiments, the pharmaceutical formulations of the disclosure comprise at least one amino acid. In some embodiments, the amino acid is arginine. In some embodiments, the arginine is provided in the form of arginine hydrochloride. In some cases, the amino acid (e.g., arginine) acts as a viscosity modifier for the anti-IL-4R antibody formulations.

The amount of amino acid contained within the pharmaceutical formulations of the present disclosure may vary depending on the specific properties desired of the formulations, as well as the particular circumstances and purposes for which the formulations are intended to be used. In certain embodiments, the formulations may contain about 1 mM to about 200 mM of an amino acid; about 5 mM to about 150 mM; about 10 mM to about 100 mM of an amino acid; about 20 mM to about 80 mM of an amino acid; about 20 mM to about 30 mM of an amino acid; about 45 mM to about 55 mM of an amino acid; or about 70 mM to about 80 mM of an amino acid. For example, the pharmaceutical formulations of the present disclosure may comprise about 5 mM; about 10 mM; about 15 mM; about 20 mM; about 25 mM; about 30 mM; about 35 mM; about 40 mM; about 45 mM; about 50 mM; about 55 mM; about 60 mM; about 65 mM; about 70 mM; about 75 mM; about 80 mM; about 85 mM; about 90 mM; about 95 mM; or about 100 mM of an amino acid (e.g., arginine). In some embodiments, the formulations contain about 25 mM of an amino acid (e.g., arginine). In some embodiments, the formulations contain about 50 mM of an amino acid (e.g., arginine). In some embodiments, the formulations contain about 75 mM of an amino acid (e.g., arginine).

### Exemplary Formulations

According to one aspect of the present disclosure, the pharmaceutical formulation comprises: (i) a human antibody that specifically binds to hIL-4R (e.g., an antibody comprising one or more sequences disclosed in Table 1 below); (ii) acetate; (iii) histidine; (iv) sucrose; (v) arginine; and (v) a surfactant comprising a polyethylene glycol or a poloxamer.

In some cases, the stable liquid pharmaceutical formulation comprises: (i) a human antibody that specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentration of from 10 mM to 15 mM; (iii) histidine at a concentration of from 15 mM to 25 mM; (iv) sucrose at a concentration of from 2.5% w/v to 7.5% w/v; (v) arginine at a concentration of from 20 mM to 80 mM; and (vi) a surfactant comprising a polyethylene glycol or a poloxamer at a concentration of from 0.01% w/v to 0.19% w/v, wherein the formulation has a pH of from 5.7 to 6.1.

In some cases, the stable liquid pharmaceutical formulation comprises: (i) a human antibody that specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentration of from 10 mM to 15 mM; (iii) histidine at a concentration of from 15 mM to 25 mM; (iv) sucrose at a concentration of from 2.5% w/v to 7.5% w/v; (v) arginine at a concentration of from 20 mM to 80 mM; and (vi) a surfactant comprising a polyethylene glycol at a concentration of from 0.01% w/v to less than 1% w/v (e.g., from 0.01% to 0.75% w/v, or from 0.01% to 0.5% w/v), wherein the formulation has a pH of from 5.7 to 6.1.

In some cases, the stable liquid pharmaceutical formulation comprises: (i) a human antibody at a concentration of from 15 mg/mL to 200 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentration of from 10 mM to 15 mM; (iii) histidine at a concentration of from 15 mM to 25 mM; (iv) sucrose at a concentration of from 2.5% w/v to 7.5% w/v; (v) arginine at a concentration of from 20 mM to 80 mM; and (vi) a surfactant comprising a polyethylene glycol or a poloxamer at a concentration of from 0.01% w/v to 0.19% w/v, wherein the formulation has a pH of from 5.7 to 6.1.

In some cases, the stable liquid pharmaceutical formulation comprises: (i) a human antibody at a concentration of from 15 mg/mL to 200 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentration of from 10 mM to 15 mM; (iii) histidine at a concentration of from 15 mM to 25 mM; (iv) sucrose at a concentration of from 2.5% w/v to 7.5% w/v; (v) arginine at a concentration of from 20 mM to 80 mM; and (vi) PEG3350 or poloxamer 188 at a concentration of from 0.01% w/v to 0.19% w/v, wherein the formulation has a pH of from 5.7 to 6.1.

In some cases, the stable liquid pharmaceutical formulation comprises: (i) a human antibody at a concentration of from 15 mg/mL to 200 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentation of 12.5 mM±1.25 mM; (iii) histidine at a concentration of 20 mM±2 mM; (iv) sucrose at a concentration of 5% w/v±0.5% w/v; (v) arginine at a concentration of 25 mM±2.5 mM; and (vi) a surfactant comprising PEG3350 at a concentration of from 0.01% w/v to 0.19% w/v or at a concentration of from 0.01% w/v to 0.5% w/v, wherein the formulation has a pH of 5.9±0.3 or a pH of 5.9±0.2.

In some cases, the stable liquid pharmaceutical formulation comprises: (i) a human antibody at a concentration of from 15 mg/mL to 200 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentation of 12.5 mM±1.25 mM; (iii) histidine at a concentration of 20 mM±2 mM; (iv) sucrose at a concentration of 5% w/v±0.5% w/v; (v) arginine at a concentration of 25 mM±2.5 mM; and (vi) a surfactant comprising poloxamer 188 at a concentration of from 0.01% w/v to 0.19% w/v, wherein the formulation has a pH of 5.9±0.3 or a pH of 5.9±0.2.

In some cases, the stable liquid pharmaceutical formulation comprises: (i) a human antibody at a concentration of from 100 mg/mL to 200 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentration of from 10 mM to 15 mM; (iii) histidine at a concentration of from 15 mM to 25 mM; (iv) sucrose at a concentration of from 2.5% w/v to 7.5% w/v; (v) arginine at a concentration of from 20 mM to 80 mM; and (vi) a surfactant comprising a polyethylene glycol or a poloxamer at a concentration of from 0.01% w/v to 0.19% w/v, wherein the formulation has a pH of from 5.7 to 6.1.

In some cases, the stable liquid pharmaceutical formulation comprises: (i) a human antibody at a concentration of from 100 mg/mL to 200 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentration of from 10 mM to 15 mM; (iii) histidine at a concentration of from 15 mM to 25 mM; (iv) sucrose at a concentration of from 2.5% w/v to 7.5% w/v; (v) arginine at a concentration of from 20 mM to 80 mM; and (vi) PEG3350 or poloxamer 188 at a concentration of from 0.01% w/v to 0.19% w/v, wherein the formulation has a pH of from 5.7 to 6.1.

In some cases, the stable liquid pharmaceutical formulation comprises: (i) a human antibody at a concentration of from 100 mg/mL to 200 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentation of 12.5 mM±1.25 mM; (iii) histidine at a concentration of 20 mM±2 mM; (iv) sucrose at a concentration of 5% w/v±0.5% w/v; (v) arginine at a concentration of 25 mM±2.5 mM; and (vi) a surfactant comprising PEG3350 at a concentration of from 0.01% w/v to 0.19% w/v or at a concentration of from 0.01% w/v to 0.5% w/v, wherein the formulation has a pH of 5.9±0.3 or a pH of 5.9±0.2.

In some cases, the stable liquid pharmaceutical formulation comprises: (i) a human antibody at a concentration of from 100 mg/mL to 200 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentation of 12.5 mM±1.25 mM; (iii) histidine at a concentration of 20 mM±2 mM; (iv) sucrose at a concentration of 5% w/v±0.5% w/v; (v) arginine at a concentration of 25 mM±2.5 mM; and (vi) a surfactant comprising poloxamer 188 at a concentration of from 0.01% w/v to 0.19% w/v, wherein the formulation has a pH of 5.9±0.3 or a pH of 5.9±0.2.

In some cases, the stable liquid pharmaceutical formulation comprises: (i) a human antibody at a concentration of 150 mg/mL±10 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentration of from 10 mM to 15 mM; (iii) histidine at a concentration of from 15 mM to 25 mM; (iv) sucrose at a concentration of from 2.5% w/v to 7.5% w/v; (v) arginine at a concentration of from 20 mM to 80 mM; and (vi) a surfactant comprising a polyethylene glycol or a poloxamer at a concentration of from 0.01% w/v to 0.19% w/v, wherein the formulation has a pH of from 5.7 to 6.1.

In some cases, the stable liquid pharmaceutical formulation comprises: (i) a human antibody at a concentration of 150 mg/mL±10 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentration of from 10 mM to 15 mM; (iii) histidine at a concentration of from 15 mM to 25 mM; (iv) sucrose at a concentration of from 2.5% w/v to 7.5% w/v; (v) arginine at a concentration of from 20 mM to 80 mM; and (vi) PEG3350 or poloxamer 188 at a concentration of from 0.01% w/v to 0.19% w/v, wherein the formulation has a pH of from 5.7 to 6.1.

In some cases, the stable liquid pharmaceutical formulation comprises: (i) a human antibody at a concentration of 150 mg/mL±10 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentation of 12.5 mM±1.25 mM; (iii) histidine at a concentration of 20 mM±2 mM; (iv) sucrose at a concentration of 5% w/v±0.5% w/v; (v) arginine at a concentration of 25 mM±2.5 mM; and (vi) a surfactant comprising PEG3350 at a concentration of from 0.01% w/v to 0.19% w/v or at a concentration of from 0.01% w/v to 0.5% w/v, wherein the formulation has a pH of 5.9±0.3 or a pH of 5.9±0.2.

In some cases, the stable liquid pharmaceutical formulation comprises: (i) a human antibody at a concentration of 150 mg/mL±10 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentation of 12.5 mM±1.25 mM; (iii) histidine at a concentration of 20 mM±2 mM; (iv) sucrose at a concentration of 5% w/v±0.5% w/v; (v) arginine at a concentration of 25 mM±2.5 mM; and (vi) a surfactant comprising poloxamer 188 at a concentration of from 0.01% w/v to 0.19% w/v, wherein the formulation has a pH of 5.9±0.3 or a pH of 5.9±0.2.

In some cases, the stable liquid pharmaceutical formulation comprises: (i) a human antibody at a concentration of 175 mg/mL±10 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentration of from 10 mM to 15 mM; (iii) histidine at a concentration of from 15 mM to 25 mM; (iv) sucrose at a concentration of from 2.5% w/v to 7.5% w/v; (v) arginine at a concentration of from 20 mM to 80 mM; and (vi) a surfactant comprising a polyethylene glycol or a poloxamer at a concentration of from 0.01% w/v to 0.19% w/v, wherein the formulation has a pH of from 5.7 to 6.1.

In some cases, the stable liquid pharmaceutical formulation comprises: (i) a human antibody at a concentration of 175 mg/mL±10 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentration of from 10 mM to 15 mM; (iii) histidine at a concentration of from 15 mM to 25 mM; (iv) sucrose at a concentration of from 2.5% w/v to 7.5% w/v; (v) arginine at a concentration of from 20 mM to 80 mM; and (vi) PEG3350 or poloxamer 188 at a concentration of from 0.01% w/v to 0.19% w/v, wherein the formulation has a pH of from 5.7 to 6.1.

In some cases, the stable liquid pharmaceutical formulation comprises: (i) a human antibody at a concentration of 175 mg/mL±10 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentation of 12.5 mM±1.25 mM; (iii) histidine at a concentration of 20 mM±2 mM; (iv) sucrose at a concentration of 5% w/v±0.5% w/v; (v) arginine at a concentration of 25 mM±2.5 mM; and (vi) a surfactant comprising PEG3350 at a concentration of from 0.01% w/v to 0.19% w/v or at a concentration of from 0.01% w/v to 0.5% w/v, wherein the formulation has a pH of 5.9±0.3 or a pH of 5.9±0.2.

In some cases, the stable liquid pharmaceutical formulation comprises: (i) a human antibody at a concentration of 175 mg/mL±10 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentation of 12.5 mM±1.25 mM; (iii) histidine at a concentration of 20 mM±2 mM; (iv) sucrose at a concentration of 5% w/v±0.5% w/v; (v) arginine at a concentration of 25 mM±2.5 mM; and (vi) a surfactant comprising poloxamer 188 at a concentration of from 0.01% w/v to 0.19% w/v, wherein the formulation has a pH of 5.9±0.3 or a pH of 5.9±0.2.

In some cases, the stable liquid pharmaceutical formulation comprises: (i) a human antibody at a concentration of 175 mg/mL±10 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentation of 12.5 mM±1.25 mM; (iii) histidine at a concentration of 20 mM±2 mM; (iv) sucrose at a concentration of 5% w/v±0.5% w/v; (v) arginine at a concentration of 50 mM±2.5 mM; and (vi) a surfactant comprising PEG3350 at a concentration of from 0.01% w/v to 0.19% w/v or at a concentration of from 0.01% w/v to 0.5% w/v, wherein the formulation has a pH of 5.9±0.3 or a pH of 5.9±0.2.

In some cases, the stable liquid pharmaceutical formulation comprises: (i) a human antibody at a concentration of 175 mg/mL±10 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentation of 12.5 mM±1.25 mM; (iii) histidine at a concentration of 20 mM±2 mM; (iv) sucrose at a concentration of 5% w/v±0.5% w/v; (v) arginine at a concentration of 50 mM±2.5 mM; and (vi) a surfactant comprising poloxamer 188 at a concentration of from 0.01% w/v to 0.19% w/v, wherein the formulation has a pH of 5.9±0.3 or a pH of 5.9±0.2.

In some cases, the stable liquid pharmaceutical formulation comprises: (i) a human antibody at a concentration of 175 mg/mL±10 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentation of 12.5 mM±1.25 mM; (iii) histidine at a concentration of 20 mM±2 mM; (iv) sucrose at a concentration of 5% w/v±0.5% w/v; (v) arginine at a concentration of 75 mM±2.5 mM; and (vi) a surfactant comprising PEG3350 at a concentration of from 0.01% w/v to 0.19% w/v or at a concentration of from 0.01% w/v to 0.5% w/v, wherein the formulation has a pH of 5.9±0.3 or a pH of 5.9±0.2.

In some cases, the stable liquid pharmaceutical formulation comprises: (i) a human antibody at a concentration of 175 mg/mL±10 mg/mL, wherein the antibody specifically binds to human IL-4Rα and comprises a heavy chain variable region (HCVR) comprising the amino acid sequence of SEQ ID NO: 1 and a light chain variable region (LCVR) comprising the amino acid sequence of SEQ ID NO: 2; (ii) acetate at a concentation of 12.5 mM±1.25 mM; (iii) histidine at a concentration of 20 mM±2 mM; (iv) sucrose at a concentration of 5% w/v±0.5% w/v; (v) arginine at a concentration of 75 mM±2.5 mM; and (vi) a surfactant comprising poloxamer 188 at a concentration of from 0.01% w/v to 0.19% w/v, wherein the formulation has a pH of 5.9±0.3 or a pH of 5.9±0.2.

In any of the various embodiments of the pharmaceutical formulations discussed above or herein, the human IL-4R antibody may comprise a human IgG1 heavy chain constant region.

In any of the various embodiments of the pharmaceutical formulations discussed above or herein, the human IL-4R antibody may comprise a human IgG4 heavy chain constant region.

In some embodiments, the human IL-4R antibody may comprise a heavy chain comprising the amino acid sequence of SEQ ID NO: 9 and a light chain comprising the amino acid sequence of SEQ ID NO: 10.

Additional non-limiting examples of pharmaceutical formulations encompassed by the present disclosure are set forth elsewhere herein, including the working Examples presented below.

### Stability of the Pharmaceutical Formulations

The pharmaceutical formulations of the present disclosure exhibit high levels of stability. The term “stable,” as used herein in reference to the pharmaceutical formulations, means that the antibodies within the pharmaceutical formulations retain an acceptable degree of structure and/or function and/or biological activity after storage for a defined amount of time. A formulation may be stable even though the antibody contained therein does not maintain 100% of its structure and/or function and/or biological activity after storage for a defined amount of time. Under certain circumstances, maintenance of about 90%, about 95%, about 96%, about 97%, about 98% or about 99% of an antibody's structure and/or function and/or biological activity after storage for a defined amount of time may be regarded as “stable.”

Stability can be measured, inter alia, by determining the percentage of antibody that forms an aggregate within the formulation after storage for a defined amount of time at a defined temperature, or under stress conditions (e.g., agitation), wherein stability is inversely proportional to the percent aggregate that is formed. The percentage of aggregated antibody can be determined by, inter alia, size exclusion chromatography (e.g., size exclusion high performance liquid chromatography [SE-HPLC] or size exclusion ultra-performance liquid chromatography [SE-UPLC]). An “acceptable degree of stability”, as that phrase is used herein, means that at most about 15%, 10%, 5%, 4%, 3%, 2.5%, 2%, 1.5%, 1%, 0.5%, or 0.1% of the antibody can be detected in an aggregate in the formulation after storage for a defined amount of time at a given temperature, or under specified stress conditions. The defined amount of time after which stability is measured can be at least 2 weeks, at least 28 days, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, at least 30 months, at least 36 months, or more. The temperature at which the pharmaceutical formulation may be stored when assessing stability can be any temperature from about −80° C. to about 45° C., e.g., storage at about −80° C., about −30° C., about −20° C., about 0° C., about 4°-8° C., about 5° C., about 25° C., about 35° C., about 37° C. or about 45° C. The “stress condition” to which the formulated antibody may be subjected may be agitation stress (e.g., vortexing) for a period of 10 minutes, 20 minutes, 30 minutes, 40 minutes, 50 minutes, 60 minutes, 70 minutes, 80 minutes, 90 minutes, 100 minutes, 110 minutes, 120 minutes, 150 minutes, 180 minutes, or more. For example, a pharmaceutical formulation comprising an anti-IL-4R antibody may be deemed stable if after nine months of storage at 5° C., less than about 2%, 1.75%, 1.5%, 1.25%, 1%, 0.75%, 0.5%, 0.25%, or 0.1% of the antibody is detected in an aggregated form. A pharmaceutical formulation may also be deemed stable if after 56 days of storage at 45° C., less than about 12% of the antibody is detected in an aggregated form. A pharmaceutical formulation may also be deemed stable if after 42 days of storage at 45° C., less than about 10% or less than about 9% of the antibody is detected in an aggregated form. A pharmaceutical formulation may also be deemed stable if after 28 days of storage at 45° C., less than about 8% or less than about 7.5% or less than about 7% of the antibody is detected in an aggregated form. A pharmaceutical formulation may also be deemed stable if after 14 days of storage at 45° C., less than about 6% of the antibody is detected in an aggregated form. A pharmaceutical formulation may also be deemed stable if after three months of storage at −20° C., −30° C., or −80° C. less than about 2%, 1.9%, 1.8%, 1.7%, 1.6%, 1.5%, 1%, 0.5%, or 0.1% of the antibody is detected in an aggregated form. A pharmaceutical formulation may also be deemed stable if after 120 minutes of agitation (e.g., via vortexing) at room temperature less than 3% or less than 2.5% of the antibody is detected in an aggregated form. In some cases, a pharmaceutical formulation may be deemed stable if it comprises no more than 4% high molecular weight (HMW) species following agitation for 24 hours at 25° C., as determined by SE-UPLC. In some cases, the formulation may be deemed stable if it comprises no more than 4% high molecular weight (HMW) species following agitation for 24 hours at 25° C. and storage at (i) 5° C. for 6, 12, or 18 months, or (ii) 25° C. for 6 months, as determined by SE-UPLC.

Stability can also be measured, inter alia, by determining particulate formulation within the formulation after storage for a defined amount of time at a defined temperature. Particle formation can be determined, for example, by microscopy techniques or by micro-flow imaging techniques.

It is known that mammalian cell lines routinely used to produce recombinant antibodies may contain enzymes that may be copurified with the antibody and be present in a pharmaceutical formulation of the antibody. In some cases, such enzymes may degrade excipients of the pharmaceutical formulation over time, and thereby lead to formation of subvisible particles (e.g., on the order of 10-25 μm or larger). For example, phospholipases are lipases that hydrolyze phospholipids into fatty acids and other products. Phospholipases fall into four broad categories: phospholipase A (including phospholipase A1 and phospholipase A2), phospholipase B, and the phosphodiesterases phosphodiesterase C and phosphodiesterase D. In addition to the canonical phospholipases, phospholipase B-like enzymes, which reside at the lysosome lumen, are thought to be involved in lipid catalysis. For example, phospholipase B-like 2 (PLBL2) is postulated to have esterase activity based upon sequence homology and subcellular localization (Jensen et al., Biochem. J. 402: 449-458 (2007)). PLBL2 is highly conserved in hamster, rat, mice, human and bovine, and may copurify under certain processes with some classes of proteins-of-interest (e.g., a therapeutic antibody such as an anti-IL-4R antibody) manufactured in a mammalian cell line. Although it is possible to remove such enzymes via purification or to include an inhibitor (e.g., an esterase inhibitor) to counteract the activity of the enzyme (as disclosed for example in U.S. Pat. No. 10,342,876), selection of excipients that prevent particle formation while also stabilizing the antibody in the pharmaceutical formulation are also of interest. Thus, in some embodiments, the formulations of the present disclosure comprise a detectable amount of a phospholipase (e.g., PLBL2). Methods of detecting and quantifying the presence and activity of phospholipase are known in the art. In some embodiments, the phospholipase is detected by immunoassay (e.g., ELISA). In some embodiments, the phospholipase is detected by liquid chromatography-mass spectrometry (LC-MS)

Accordingly, a pharmaceutical formulation (containing a phospholipase) may be deemed stable if after storage for a period of time (e.g., 6, 12, 18, 24 or 36 months or more) at a defined temperature (e.g., 5° C.), no more than a specified number of particles (e.g., 3000 particles, 1000 particles, 500 particles, 250 particles, 100 particles, or 50 particles per container for 10 micron) are identified within a volume of 2.25 mL. For example, a pharmaceutical formulation may be deemed stable if after 24 months of storage at 5° C. no more than 1000 particles are identified within a volume of 2.25 mL via microscopy. A pharmaceutical formulation may also be deemed stable if after 24 months of storage at 5° C. no more than 500 particles are identified within a volume of 2.25 mL via microscopy. A pharmaceutical formulation may also be deemed stable if after 24 months of storage at 5° C. no more than 250 particles are identified within a volume of 2.25 mL via microscopy. A pharmaceutical formulation may also be deemed stable if after 24 months of storage at 5° C. no more than 150 particles are identified within a volume of 2.25 mL via microscopy. A pharmaceutical formulation may also be deemed stable if after 36 months of storage at 5° C. no more than 1000 particles are identified within a volume of 2.25 mL via microscopy. A pharmaceutical formulation may also be deemed stable if after 36 months of storage at 5° C. no more than 500 particles are identified within a volume of 2.25 mL via microscopy. A pharmaceutical formulation may also be deemed stable if after 36 months of storage at 5° C. no more than 250 particles are identified within a volume of 2.25 mL via microscopy. A pharmaceutical formulation may also be deemed stable if after 36 months of storage at 5° C. no more than 150 particles are identified within a volume of 2.25 mL via microscopy. A pharmaceutical formulation may also be deemed stable if after 36 months of storage at 5° C. no more than 100 particles are identified within a volume of 2.25 mL via microscopy. A pharmaceutical formulation may also be deemed stable if after 36 months of storage at 5° C. no more than 50 particles are identified within a volume of 2.25 mL via microscopy.

Stability can also be measured by, inter alia, determining the percentage of native antibody remaining in the formulation after storage for a defined amount of time at a given temperature. The percentage of native antibody can be determined by, inter alia, size exclusion chromatography (e.g., size exclusion high performance liquid chromatography [SE-HPLC]). An “acceptable degree of stability,” as that phrase is used herein, means that at least 90% of the native form of the antibody can be detected in the formulation after storage for a defined amount of time at a given temperature. In certain embodiments, at least about 90%, 91%, 92%, 93%, 94%, 95%, 96%, 97%, 98%, 99% or 100% of the native form of the antibody can be detected in the formulation after storage for a defined amount of time at a given temperature. The defined amount of time after which stability is measured can be at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, at least 30 months, at least 36 months, or more. The temperature at which the pharmaceutical formulation may be stored when assessing stability can be any temperature from about −80° C. to about 45° C., e.g., storage at about −80° C., about −30° C., about −20° C., about 0° C., about 2°−8° C., 4°−8° C., about 5° C., about 25° C., about 35° C., about 37° C., or about 45° C.

Stability can also be measured, inter alia, by determining the percentage of antibody that migrates in a more acidic fraction during ion exchange (“acidic form”) than in the main fraction of antibody (“main charge form”), wherein stability is inversely proportional to the fraction of antibody in the acidic form. While not wishing to be bound by theory, deamidation of the antibody may cause the antibody to become more negatively charged and thus more acidic relative to the non-deamidated antibody (see, e.g., Robinson, N., Protein Deamidation, PNAS, Apr. 16, 2002, 99(8):5283-5288). The percentage of “acidified” antibody can be determined by ion exchange chromatography (e.g., cation exchange high performance liquid chromatography [CEX-HPLC] or cation exchange ultra-performance liquid chromatography [CEX-UPLC]). An “acceptable degree of stability”, as that phrase is used herein, means that at most 50%, 45%, 40%, 35%, 30%, 25%, 20%, 15%, 10%, 5%, 4%, 3%, 2%, 1%, 0.5%, or 0.1% of the antibody can be detected in an acidic form in the formulation after storage for a defined amount of time at a given temperature. The defined amount of time after which stability is measured can be at least 2 weeks, at least 28 days, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, at least 30 months, at least 36 months, or more. The temperature at which the pharmaceutical formulation may be stored when assessing stability can be any temperature from about −80° C. to about 45° C., e.g., storage at about −80° C., about −30° C., about −20° C., about 0° C., about 4°-8° C., about 5° C., about 25° C., or about 45° C.

Measuring the binding affinity of the antibody to its target may also be used to assess stability. For example, a formulation of the present disclosure may be regarded as stable if, after storage at e.g., −80° C., −30° C., −20° C., 5° C., 25° C., 37° C., 45° C., etc. for a defined amount of time (e.g., 14 days to 9 months), the anti-IL-4R antibody contained within the formulation binds to hIL-4Rα with an affinity that is at least 80%, 85%, 90%, 95%, or more of the binding affinity of the antibody prior to said storage. Binding affinity may be determined by any method, such as e.g., ELISA or plasmon resonance. Biological activity may be determined by, for example, measuring the downstream activity of the IL-4R system in the presence of the antibody, and comparing the activity to the activity of the IL-4R system in the absence of antibody.

References to stability of the pharmaceutical formulations “after” a specified period of time are intended to mean that a measurement of a stability parameter (e.g., % native form, % HMW species, or % acidic form) is taken at or about the end of the specific time period, and is not intended to mean that the pharmaceutical formulation necessarily maintains the same degree of stability for the measured parameter thereafter. For example, reference to a particular stability after 12 months means that the measurement of stability was taken at or about 12 months after the start of the study. Additional methods for assessing the stability of an antibody in formulation are demonstrated in the Examples presented below.

As illustrated in the Examples below, the present disclosure is based, in part, on the discovery that the combination of claimed excipients with an anti-IL-4R antibody produces a formulation that is stable over long periods (e.g., 36 months) of storage.

### Containers and Methods of Administration

The pharmaceutical formulations of the present disclosure may be contained within any container suitable for storage of medicines and other therapeutic compositions. For example, the pharmaceutical formulations may be contained within a sealed and sterilized plastic or glass container having a defined volume such as a vial, ampule, syringe, cartridge, bottle or IV bag. Different types of vials can be used to contain the formulations of the present disclosure including, e.g., clear and opaque (e.g., amber) glass or plastic vials. Likewise, any type of syringe can be used to contain and/or administer the pharmaceutical formulations of the present disclosure. In some embodiments, the pharmaceutical formulation is contained in a prefilled syringe. In some embodiments, the pharmaceutical formulation is contained in a prefilled staked needle syringe.

The pharmaceutical formulations of the present disclosure may be contained within “normal tungsten” syringes or “low tungsten” syringes. As will be appreciated by persons of ordinary skill in the art, the process of making glass syringes generally involves the use of a hot tungsten rod which functions to pierce the glass thereby creating a hole from which liquids can be drawn and expelled from the syringe. This process results in the deposition of trace amounts of tungsten on the interior surface of the syringe. Subsequent washing and other processing steps can be used to reduce the amount of tungsten in the syringe. As used herein, the term “normal tungsten” means that the syringe contains greater than 500 parts per billion (ppb) of tungsten. The term “low tungsten” means that the syringe contains less than 500 ppb of tungsten. For example, a low tungsten syringe, according to the present disclosure, can contain less than about 490, 480, 470, 460, 450, 440, 430, 420, 410, 390, 350, 300, 250, 200, 150, 100, 90, 80, 70, 60, 50, 40, 30, 20, 10 or fewer ppb of tungsten.

The rubber plungers used in syringes, and the rubber stoppers used to close the openings of vials, may be coated to prevent contamination of the medicinal contents of the syringe or vial and/or to preserve their stability. Thus, pharmaceutical formulations of the present disclosure, according to certain embodiments, may be contained within a syringe that comprises a coated plunger, or within a vial that is sealed with a coated rubber stopper. For example, the plunger or stopper may be coated with a fluorocarbon film. Examples of coated stoppers and/or plungers suitable for use with vials and syringes containing the pharmaceutical formulations of the present disclosure are mentioned in, e.g., U.S. Pat. Nos. 4,997,423; 5,908,686; 6,286,699; 6,645,635; and 7,226,554, the contents of which are incorporated by reference herein in their entireties. Particular exemplary coated rubber stoppers and plungers that can be used in the context of the present disclosure are commercially available under the tradename “FluroTec®,” available from West Pharmaceutical Services, Inc. (Lionville, PA). According to certain embodiments of the present disclosure, the pharmaceutical formulations may be contained within a low tungsten syringe that comprises a fluorocarbon-coated plunger. In some embodiments, the container is a syringe, such as an Ompi EZ-Fill™ syringe or a BD Neopak™ syringe. In some cases, the syringe is a 1 mL long glass syringe with a 1 mL iWest piston, a 27G thin wall needle and an FM30 needle shield or a BD260 needle shield. In some cases, the syringe is a 2.25 mL glass syringe (e.g., Nuova Ompi). In various embodiments, the syringe is a 0.5 mL, 0.6 mL, 0.7 mL, 0.8 mL, 0.9 mL, 1.0 mL, 1.1 mL, 1.2 mL, 1.3 mL, 1.4 mL, 1.5 mL, 1.6 mL, 1.7 mL, 1.8 mL, 1.9 mL, 2.0 mL, 2.1 mL, 2.2 mL, 2.3 mL, 2.4 mL, 2.5 mL, 2.6 mL, 2.7 mL, 2.8 mL, 2.9 mL, 3.0 mL, 3.5 mL, 4.0 mL, 4.5 mL, 5.0 mL, 5.5 mL, 6.0 mL, 6.5 mL, 7.0 mL, 7.5 mL, 8.0 mL, 8.5 mL, 9.0 mL, 9.5 mL, or 10 mL syringe (e.g., a glass syringe).

The pharmaceutical formulations can be administered to a patient by parenteral routes such as injection (e.g., subcutaneous, intravenous, intramuscular, intraperitoneal, etc.) or percutaneous, mucosal, nasal, pulmonary and/or oral administration. Numerous reusable pen and/or autoinjector delivery devices can be used to subcutaneously deliver the pharmaceutical formulations of the present disclosure. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (sanofi-aventis, Frankfurt, Germany), to name only a few. Examples of disposable pen and/or autoinjector delivery devices having applications in subcutaneous delivery of a pharmaceutical composition of the present disclosure include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L. P.), and the HUMIRA™ Pen (Abbott Labs, Abbott Park, IL), to name only a few. In some cases, the pharmaceutical formulation is contained in a syringe specifically adapted for use with an autoinjector. Subcutaneous injections may be administered using a 20-30 gauge needle, or a 25-30 gauge needle. In some cases, subcutaneous injections may be administered using a 25 gauge needle. In some cases, subcutaneous injections may be administered using a 27 gauge needle. In some cases, subcutaneous injections may be administered using a 29 gauge needle.

Another type of delivery device can include a safety system. Such devices can be relatively inexpensive, and operate to manually or automatically extend a safety sleeve over a needle once injection is complete. Examples of safety systems can include the ERIS device by West Pharmaceutical, or the UltraSafe device by Becton Dickinson. In addition, the use of a large volume device (“LVD”), or bolus injector, to deliver the pharmaceutical formulations of the present disclosure is also contemplated herein. In some cases, the LVD or bolus injector may be configured to inject a medicament into a patient. For example, an LVD or bolus injector may be configured to deliver a “large” volume of medicament (typically about 2 mL to about 10 mL).

In some embodiments, a pharmaceutical composition comprising an anti-IL-4R antibody is administered using a drug delivery device that is a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

An exemplary sleeve-triggered auto-injector with manual needle insertion is described in International Publication WO2015/004052. Exemplary audible end-of-dose feedback mechanisms are described in International Publications WO2016/193346 and WO2016/193348. An exemplary needle-safety mechanism after using an auto-injector is described in International Publication WO2016/193352. An exemplary needle sheath remover mechanism for a syringe auto-injector is described in International Publication WO2016/193353. An exemplary support mechanism for supporting an axial position of a syringe is described in International Publication WO2016/193355.

The pharmaceutical formulations of the present disclosure can also be contained in a unit dosage form. The term “unit dosage form,” as used herein, refers to a physically discrete unit suitable as a unitary dosage for the patient to be treated, each unit containing a predetermined quantity of active compound calculated to produce the desired therapeutic effect in association with the required pharmaceutical carrier, diluent, or excipient. In various embodiments, the unit dosage form is contained within a container as discussed herein. Actual dosage levels of the active ingredient (e.g., an anti-IL-4R antibody) in the formulations of the present disclosure may be varied so as to obtain an amount of the active ingredient which is effective to achieve the desired therapeutic response for a particular patient, composition, and mode of administration, without adverse effect to the patient. The selected dosage level will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. The term “diluent” as used herein refers to a solution suitable for altering or achieving an exemplary or appropriate concentration or concentrations as described herein.

In various embodiments, the unit dosage form contains an amount of the active ingredient (e.g., an anti-IL-4R antibody) intended for a single use. In various embodiments, the amount of the active ingredient in the unit dosage form is from about 0.1 mg to about 5000 mg, from about 100 mg to about 1000 mg, and from about 100 mg to about 500 mg, from about 100 mg to about 400 mg, from about 100 mg to about 200 mg, from about 250 mg to about 350 mg, from about 125 mg to about 175 mg, from about 275 mg to about 325 mg, or ranges or intervals thereof. For example, ranges of values using a combination of any of the above recited values (or values contained within the above recited ranges) as upper and/or lower limits are intended to be included. In a particular embodiment, the formulation often is supplied as a liquid in unit dosage form. In some embodiments, the unit dosage form contains about 100 mg of the active ingredient. In some embodiments, the unit dosage form contains about 150 mg. In some embodiments, the unit dosage form contains about 200 mg. In some embodiments, the unit dosage form contains about 300 mg. In some embodiments, the unit dosage form contains about 350 mg. In some embodiments, the unit dosage form contains about 600 mg. In some embodiments, a unit dosage form according to the present disclosure is suitable for subcutaneous administration to a patient.

The present disclosure also includes methods of preparing a unit dosage form. In an exemplary embodiment, a method for preparing a pharmaceutical unit dosage form includes combining the formulation of any of foregoing embodiments in a suitable container (e.g., those containers discussed herein).

### Therapeutic Uses of the Pharmaceutical Formulations

The pharmaceutical formulations of the present disclosure are useful, inter alia, for the treatment, prevention and/or amelioration of any disease or disorder associated with IL-4R activity.

The therapeutic methods of the present disclosure comprise administering to a subject any formulation comprising an anti-hIL-4R antibody as disclosed herein. The subject to which the pharmaceutical formulation is administered can be, e.g., any human or non-human animal that is in need of such treatment, prevention and/or amelioration, or who would otherwise benefit from the inhibition or attenuation of IL-4R and/or IL-4R-mediated activity. The present disclosure further includes the use of any of the pharmaceutical formulations disclosed herein in the manufacture of a medicament for the treatment, prevention and/or amelioration of any disease or disorder associated with IL-4R activity.

In some embodiments, the disease or disorder associated with IL-4R activity is an inflammatory condition, allergic condition, lung/respiratory disorder, gastrointestinal disorder, or dermatological disorder. In some embodiments, the disease or disorder is a Type 2 inflammatory disorder. In some embodiments, the disease or disorder is an atopic disease. Non-limiting examples of diseases and disorders associated with IL-4R activity include allergy (e.g., food allergy or environmental allergy), allergic reactions, allergic bronchopulmonary aspergillosis, alopecia areata, asthma (including mild, moderate, or severe asthma, persistent asthma, or allergic asthma), arthritis (including septic arthritis), atopic dermatitis (including moderate or severe atopic dermatitis), atopic keratoconjunctivitis, autoimmune hemolytic anemia, autoimmune lymphoproliferative syndrome, autoimmune uveitis, Barrett's esophagus, benign prostate hyperplasia, bronchiectasis, bullous pemphigoid, Churg-Strauss syndrome, chronic idiopathic urticaria, chronic inducible urticaria, cold inducible urticaria, contact dermatitis (e.g., allergic contact dermatitis), COPD, eosinophilic esophagitis, eosinophilic duodenitis, eosinophilic gastroenteritis, Grave's disease, herpetiformis, hypertrophic scarring, inflammatory bowel disease, Kawasaki disease, nasal polyposis, nephrosis, Netherton's syndrome, pre-eclampsia, prurigo nodularis, pruritus (e.g., chronic pruritus of unknown origin), rhinitis (e.g., allergic rhinitis), rhinosinusitis (e.g., allergic fungal rhinosinusitis, chronic rhinosinusitis with or without nasal polyposis), scleroderma, sickle cell disease, Sjogren's syndrome, tuberculosis, ulcerative colitis, and Whipple's Disease.

In some embodiments, the present disclosure provides kits comprising a pharmaceutical formulation (e.g., a container with the formulation or a unit dosage form), as discussed herein, and packaging or labeling (e.g., a package insert) with instructions to use the pharmaceutical formulation for the treatment of a disease or disorder, as discussed above. In some cases, the instructions provide for use of a unit dosage form, as discussed herein, for the treatment of a disease or disorder.

A summary of the sequences and the corresponding SEQ ID NOs referenced herein is shown in Table 1, below.

TABLE 1
Informal Sequence Listing

| SEQ | | |
|---|---|---|
| ID | | |
| NO | Sequence | Description |
| 1 | EVQLVESGGGLEQPGGSLRLSCAGSGFTFRDYAMTWVRQAPGKGLEWVSSISGSG | Dupilumab |
| | GNTYYADSVKGRFTISRDNSKNTLYLQMNSLRAEDTAVYYCAKDRLSITIRPRYYGLD | HCVR |
| | VWGQGTTVTVS | amino acid |
| | | sequence |
| 2 | DIVMTQSPLSLPVTPGEPASISCRSSQSLLYSIGYNYLDWYLQKSGQSPQLLIYLGSNR | Dupilumab |
| | ASGVPDRFSGSGSGTDFTLKISRVEAEDVGFYYCMQALQTPYTFGQGTKLEIK | LCVR |
| | | amino acid |
| | | sequence |
| 3 | GFTFRDYA | Dupilumab |
| | | HCDR1 |
| | | amino acid |
| | | sequence |
| 4 | ISGSGGNT | Dupilumab |
| | | HCDR2 |
| | | amino acid |
| | | sequence |
| 5 | AKDRLSITIRPRYYGLDV | Dupilumab |
| | | HCDR3 |
| | | amino acid |
| | | sequence |
| 6 | QSLLYSIGYNY | Dupilumab |
| | | LCDR1 |
| | | amino acid |
| | | sequence |
| 7 | LGS | Dupilumab |
| | | LCDR2 |
| | | amino acid |
| | | sequence |
| 8 | MQALQTPYT | Dupilumab |
| | | LCDR3 |
| | | amino acid |
| | | sequence |
| 9 | EVQLVESGGGLEQPGGSLRLSCAGSGFTFRDYAMTWVRQAPGKGLEWVSSISGSG | Dupilumab |
| | GNTYYADSVKGRFTISRDNSKNTLYLQMNSLRAEDTAVYYCAKDRLSITIRPRYYGLD | heavy |
| | VWGQGTTVTVSSASTKGPSVFPLAPCSRSTSESTAALGCLVKDYFPEPVTVSWNSGA | chain |
| | LTSGVHTFPAVLQSSGLYSLSSVVTVPSSSLGTKTYTCNVDHKPSNTKVDKRVESKYGP | amino acid |
| | PCPPCPAPEFLGGPSVFLFPPKPKDTLMISRTPEVTCVVVDVSQEDPEVQFNWYVDG | sequence |
| | VEVHNAKTKPREEQFNSTYRVVSVLTVLHQDWLNGKEYKCKVSNKGLPSSIEKTISKA | |
| | KGQPREPQVYTLPPSQEEMTKNQVSLTCLVKGFYPSDIAVEWESNGQPENNYKTTP | |
| | PVLDSDGSFFLYSRLTVDKSRWQEGNVFSCSVMHEALHNHYTQKSLSLSLGK | |
| 10 | DIVMTQSPLSLPVTPGEPASISCRSSQSLLYSIGYNYLDWYLQKSGQSPQLLIYLGSNR | Dupilumab |
| | ASGVPDRFSGSGSGTDFTLKISRVEAEDVGFYYCMQALQTPYTFGQGTKLEIKRTVAA | light |
| | PSVFIFPPSDEQLKSGTASVVCLLNNFYPREAKVQWKVDNALQSGNSQESVTEQDSK | chain |
| | DSTYSLSSTLTLSKADYEKHKVYACEVTHQGLSSPVTKSFNRGEC | amino acid |
| | | sequence |
| 11 | MKVLQEPTCVSDYMSISTCEWKMNGPTNCSTELRLLYQLVFLLSEAHTCIPENNGGA | Human |
| | GCVCHLLMDDVVSADNYTLDLWAGQQLLWKGSFKPSEHVKPRAPGNLTVHTNVS | IL-4Rα |
| | DTLLLTWSNPYPPDNYLYNHLTYAVNIWSENDPADFRIYNVTYLEPSLRIAASTLKSGI | |
| | SYRARVRAWAQCYNTTWSEWSPSTKWHNSYREPFEQH | |
| 12 | EIVLTQSPGTLSLSPGERATLSCRASQSVSNSYLAWYQQKPGQAPRLLIFGASSRATGI | SCB-VL-39 |
| | PDRFSGSGSGTDFTLTISRLEPEDFAVYYCQQYGSSPPWTFGQGTKVEIK | |
| 13 | EIVLTQSPGTLSLSPGERATLSCRASQSVSSSYLAWYQQKPGQAPRLLIYGASSRATGI | SCB-VL-40 |
| | PDRFSGSGSGTDFTLTISRLEPEDFAVYYCQQYGSSPPWTFGQGTKVEIK | |
| 14 | EIVLTQSPGTLSLSPGERATLSCRASQSVSSSYLAWYQQKPGQAPRLLIFGASSRAPGI | SCB-VL-41 |
| | PDRFSGSGSGTDFTLTISRLEPEDFAVYYCQQYGSSPPWTFGQGTKVEIK | |
| 15 | EIVLTQSPGTLSLSPGERATLSCRASQSVSNSYLAWYQQKPGQAPRLLIYGASSRATGI | SCB-VL-42 |
| | PDRFSGSGSGTDFTLTISRLEPEDFAVYYCQQYGSSPPWTFGQGTKVEIK | |
| 16 | EIVLTQSPGTLSLSPGERATLSCRASQSVSNSYLAWYQQKPGQAPRLLIFGASSRAPGI | SCB-VL-43 |
| | PDRFSGSGSGTDFTLTISRLEPEDFAVYYCQQYGSSPPWTFGQGTKVEIK | |
| 17 | EIVLTQSPGTLSLSPGERATLSCRASQSVSSSYLAWYQQKPGQAPRLLIYGASSRAPGI | SCB-VL-44 |
| | PDRFSGSGSGTDFTLTISRLEPEDFAVYYCQQYGSSPPWTFGQGTKVEIK | |
| 18 | EIVLTQSPGTLSLSPGERATLSCRASQSVSSSYLAWYQQKPGQAPRLLIFGASSRATGI | SCB-VL-45 |
| | PDRFSGSGSGTDFTLTISRLEPEDFAVYYCQQYDHSPPWTFGQGTKVEIK | |
| 19 | EIVLTQSPGTLSLSPGERATLSCRASQSVSSSYLAWYQQKPGQAPRLLIFGASSRATGI | SCB-VL-46 |
| | PDRFSGSGSGTDFTLTISRLEPEDFAVYYCQQYGSSAGWTFGQGTKVEIK | |
| 20 | EIVLTQSPGTLSLSPGERATLSCRASQSVSSSYLAWYQQKPGQAPRLLIFGASSRATGI | SCB-VL-47 |
| | PDRFSGSGSGTDFTLTISRLEPEDFAVYYCQQYDHSAGWTFGQGTKVEIK | |
| 21 | EIVLTQSPGTLSLSPGERATLSCRASQSVSNSYLAWYQQKPGQAPRLLIFGASSRATGI | SCB-VL-48 |
| | PDRFSGSGSGTDFTLTISRLEPEDFAVYYCQQYDHSPPWTFGQGTKVEIK | |
| 22 | EIVLTQSPGTLSLSPGERATLSCRASQSVSSSYLAWYQQKPGQAPRLLIYGASSRATGI | SCB-VL-49 |
| | PDRFSGSGSGTDFTLTISRLEPEDFAVYYCQQYDHSPPWTFGQGTKVEIK | |
| 23 | EIVLTQSPGTLSLSPGERATLSCRASQSVSSSYLAWYQQKPGQAPRLLIFGASSRAPGI | SCB-VL-50 |
| | PDRFSGSGSGTDFTLTISRLEPEDFAVYYCQQYDHSPPWTFGQGTKVEIK | |
| 24 | EIVLTQSPGTLSLSPGERATLSCRASQSVSSSYLAWYQQKPGQAPRLLIYGASSRAPGI | SCB-VL-51 |
| | PDRFSGSGSGTDFTLTISRLEPEDFAVYYCQQYDHSAGWTFGQGTKVEIK | |
| 25 | EIVLTQSPGTLSLSPGERATLSCRASQSVSNSYLAWYQQKPGQAPRLLIFGASSRAPGI | SCB-VL-52 |
| | PDRFSGSGSGTDFTLTISRLEPEDFAVYYCQQYDHSAGWTFGQGTKVEIK | |
| 26 | EIVLTQSPGTLSLSPGERATLSCRASQSVSNSYLAWYQQKPGQAPRLLIYGASSRATGI | SCB-VL-53 |
| | PDRFSGSGSGTDFTLTISRLEPEDFAVYYCQQYDHSAGWTFGQGTKVEIK | |
| 27 | EIVLTQSPGTLSLSPGERATLSCRASQSVSSSYLAWYQQKPGQAPRLLIFGASSRAPGI | SCB-VL-54 |
| | PDRFSGSGSGTDFTLTISRLEPEDFAVYYCQQYDHSAGWTFGQGTKVEIK | |
| 28 | EIVLTQSPGTLSLSPGERATLSCRASQSVSSSYLAWYQQKPGQAPRLLIYGASSRATGI | SCB-VL-55 |
| | PDRFSGSGSGTDFTLTISRLEPEDFAVYYCQQYDHSAGWTFGQGTKVEIK | |
| 29 | EIVLTQSPGTLSLSPGERATLSCRASQSVSNSYLAWYQQKPGQAPRLLIFGASSRATGI | SCB-VL-56 |
| | PDRFSGSGSGTDFTLTISRLEPEDFAVYYCQQYDHSAGWTFGQGTKVEIK | |
| 30 | EIVLTQSPGTLSLSPGERATLSCRASQSVSSSYLAWYQQKPGQAPRLLIFGASSRATGI | SCB-VL-57 |
| | PDRFSGSGSGTDFTLTISRLEPEDFAVYYCQQYGSSPPWTFGQGTKVEIK | |
| 31 | EIVLTQSPGTLSLSPGERATLSCRASQSVSNSYLAWYQQKPGQAPRLLIYGASSRAPGI | SCB-VL-58 |
| | PDRFSGSGSGTDFTLTISRLEPEDFAVYYCQQYDHSAGWTFGQGTKVEIK | |
| 32 | EVQLVESGGGLVHPGGSLRLSCAGSGFTFSRNAMFWVRQAPGKGLEWVSGIGTGG | SCB-VH-59 |
| | ATNYADSVKGRFTISRDNAKNSLYLQMNSLRAEDMAVYYCARGRYYFDYWGQGTL | |
| | VTVSS | |
| 33 | EVQLVQSGGGLVQPGGSLRLSCAGSGFTFSRNAMFWVRQAPGKGLEWVSGIGTG | SCB-VH-60 |
| | GATNYADSVKGRFTISRDNAKNSLYLQMNSLRAEDMAVYYCARGRYYFDYWGQGT | |
| | LVTVSS | |
| 34 | EVQLVQSGGGLVHPGGSLRLSCAASGFTFSRNAMFWVRQAPGKGLEWVSGIGTGG | SCB-VH-61 |
| | ATNYADSVKGRFTISRDNAKNSLYLQMNSLRAEDMAVYYCARGRYYFDYWGQGTL | |
| | VTVSS | |
| 35 | EVQLVQSGGGLVHPGGSLRLSCAGSGFTFSRNAMFWVRQAPGKGLEWVSGIGTGG | SCB-VH-62 |
| | ATSYADSVKGRFTISRDNAKNSLYLQMNSLRAEDMAVYYCARGRYYFDYWGQGTLV | |
| | TVSS | |
| 36 | EVQLVQSGGGLVHPGGSLRLSCAGSGFTFSRNAMFWVRQAPGKGLEWVSGIGTGG | SCB-VH-63 |
| | ATNYADSVKGRFTISRDNAKNSLYLQMNSLRAEDTAVYYCARGRYYFDYWGQGTLV | |
| | TVSS | |
| 37 | EVQLVESGGGLVQPGGSLRLSCAGSGFTFSRNAMFWVRQAPGKGLEWVSGIGTGG | SCB-VH-64 |
| | ATNYADSVKGRFTISRDNAKNSLYLQMNSLRAEDMAVYYCARGRYYFDYWGQGTL | |
| | VTVSS | |
| 38 | EVQLVESGGGLVHPGGSLRLSCAASGFTFSRNAMFWVRQAPGKGLEWVSGIGTGG | SCB-VH-65 |
| | ATNYADSVKGRFTISRDNAKNSLYLQMNSLRAEDMAVYYCARGRYYFDYWGQGTL | |
| | VTVSS | |
| 39 | EVQLVQSGGGLVQPGGSLRLSCAASGFTFSRNAMFWVRQAPGKGLEWVSGIGTGG | SCB-VH-66 |
| | ATNYADSVKGRFTISRDNAKNSLYLQMNSLRAEDMAVYYCARGRYYFDYWGQGTL | |
| | VTVSS | |
| 40 | EVQLVQSGGGLVHPGGSLRLSCAGSGFTFSRNAMFWVRQAPGKGLEWVSGIGTGG | SCB-VH-67 |
| | ATSYADSVKGRFTISRDNAKNSLYLQMNSLRAEDTAVYYCARGRYYFDYWGQGTLV | |
| | TVSS | |
| 41 | EVQLVQSGGGLVHPGGSLRLSCAGSGFTFSRNAMFWVRQAPGKGLEWVSGIGTGG | SCB-VH-68 |
| | ATNYADSVKGRFTISRDNAKNSLYLQMNSLRAEDMAVYYCARGRYYFPWWGQGTL | |
| | VTVSS | |
| 42 | EVQLVESGGGLVHPGGSLRLSCAGSGFTFSRNAMFWVRQAPGKGLEWVSGIGTGG | SCB-VH-69 |
| | ATNYADSVKGRFTISRDNAKNSLYLQMNSLRAEDMAVYYCARGRYYFPWWGQGTL | |
| | VTVSS | |
| 43 | EVQLVQSGGGLVQPGGSLRLSCAGSGFTFSRNAMFWVRQAPGKGLEWVSGIGTG | SCB-VH-70 |
| | GATNYADSVKGRFTISRDNAKNSLYLQMNSLRAEDMAVYYCARGRYYFPWWGQG | |
| | TLVTVSS | |
| 44 | EVQLVQSGGGLVHPGGSLRLSCAASGFTFSRNAMFWVRQAPGKGLEWVSGIGTGG | SCB-VH-71 |
| | ATNYADSVKGRFTISRDNAKNSLYLQMNSLRAEDMAVYYCARGRYYFPWWGQGTL | |
| | VTVSS | |
| 45 | EVQLVQSGGGLVHPGGSLRLSCAGSGFTFSRNAMFWVRQAPGKGLEWVSGIGTGG | SCB-VH-72 |
| | ATSYADSVKGRFTISRDNAKNSLYLQMNSLRAEDMAVYYCARGRYYFPWWGQGTL | |
| | VTVSS | |
| 46 | EVQLVQSGGGLVHPGGSLRLSCAGSGFTFSRNAMFWVRQAPGKGLEWVSGIGTGG | SCB-VH-73 |
| | ATNYADSVKGRFTISRDNAKNSLYLQMNSLRAEDTAVYYCARGRYYFPWWGQGTL | |
| | VTVSS | |
| 47 | EVQLVQSGGGLVHPGRSLRLSCAGSGFTFSRNAMFWVRQAPGKGLEWVSGIGTGG | SCB-VH-74 |
| | ATNYADSVKGRFTISRDNAKNSLYLQMNSLRAEDMAVYYCARGRYYFDYWGQGTL | |
| | VTVSS | |
| 48 | EVQLVQSGGGLVHPGGSLRLTCAGSGFTFSRNAMFWVRQAPGKGLEWVSGIGTG | SCB-VH-75 |
| | GATNYADSVKGRFTISRDNAKNSLYLQMNSLRAEDMAVYYCARGRYYFDYWGQGT | |
| | LVTVSS | |
| 49 | EVQLVQSGGGLVHPGGSLRLSCAGSGFTFSRNAMHWVRQAPGKGLEWVSGIGTG | SCB-VH-76 |
| | GATNYADSVKGRFTISRDNAKNSLYLQMNSLRAEDMAVYYCARGRYYFDYWGQGT | |
| | LVTVSS | |
| 50 | EVQLVQSGGGLVHPGGSLRLSCAGSGFTFSRNAMFWVRQAPGEGLEWVSGIGTGG | SCB-VH-77 |
| | ATNYADSVKGRFTISRDNAKNSLYLQMNSLRAEDMAVYYCARGRYYFDYWGQGTL | |
| | VTVSS | |
| 51 | EVQLVQSGGGLVHPGGSLRLSCAGSGFTFSRNAMFWVRQAPGKGLEWVSGIGTGG | SCB-VH-78 |
| | ATNYADSVKGRFTISRDEAKNSLYLQMNSLRAEDMAVYYCARGRYYFDYWGQGTLV | |
| | TVSS | |
| 52 | EVQLVQSGGGLVHPGGSLRLSCAGSGFTFSRNAMFWVRQAPGKGLEWVSGIGTGG | SCB-VH-79 |
| | ATNYADSVKGRFTISRDNAKNSLYLQMNSLRAGDMAVYYCARGRYYFDYWGQGTL | |
| | VTVSS | |
| 53 | EVQLVQSGGGLVHPGGSLRLSCAGSGFTFDDYAMFWVRQAPGKGLEWVSGIGTG | SCB-VH-80 |
| | GATNYADSVKGRFTISRDNAKNSLYLQMNSLRAEDMAVYYCARGRYYFDYWGQGT | |
| | LVTVSS | |
| 54 | EVQLVQSGGGLVQPGGSLRLSCAASGFTFSRNAMFWVRQAPGKGLEWVSGIGTGG | SCB-VH-81 |
| | ATSYADSVKGRFTISRDNAKNSLYLQMNSLRAEDTAVYYCARGRYYFPWWGQGTLV | |
| | TVSS | |
| 55 | EVQLVESGGGLVHPGGSLRLSCAASGFTFSRNAMFWVRQAPGKGLEWVSGIGTGG | SCB-VH-82 |
| | ATSYADSVKGRFTISRDNAKNSLYLQMNSLRAEDTAVYYCARGRYYFPWWGQGTLV | |
| | TVSS | |
| 56 | EVQLVESGGGLVQPGGSLRLSCAGSGFTFSRNAMFWVRQAPGKGLEWVSGIGTGG | SCB-VH-83 |
| | ATSYADSVKGRFTISRDNAKNSLYLQMNSLRAEDTAVYYCARGRYYFPWWGQGTLV | |
| | TVSS | |
| 57 | EVQLVESGGGLVQPGGSLRLSCAASGFTFSRNAMFWVRQAPGKGLEWVSGIGTGG | SCB-VH-84 |
| | ATNYADSVKGRFTISRDNAKNSLYLQMNSLRAEDTAVYYCARGRYYFPWWGQGTL | |
| | VTVSS | |
| 58 | EVQLVESGGGLVQPGGSLRLSCAASGFTFSRNAMFWVRQAPGKGLEWVSGIGTGG | SCB-VH-85 |
| | ATSYADSVKGRFTISRDNAKNSLYLQMNSLRAEDMAVYYCARGRYYFPWWGQGTL | |
| | VTVSS | |
| 59 | EVQLVQSGGGLVHPGGSLRLSCAASGFTFSRNAMFWVRQAPGKGLEWVSGIGTGG | SCB-VH-86 |
| | ATSYADSVKGRFTISRDNAKNSLYLQMNSLRAEDTAVYYCARGRYYFPWWGQGTLV | |
| | TVSS | |
| 60 | EVQLVQSGGGLVQPGGSLRLSCAGSGFTFSRNAMFWVRQAPGKGLEWVSGIGTG | SCB-VH-87 |
| | GATSYADSVKGRFTISRDNAKNSLYLQMNSLRAEDTAVYYCARGRYYFPWWGQGTL | |
| | VTVSS | |
| 61 | EVQLVESGGGLVHPGGSLRLSCAGSGFTFSRNAMFWVRQAPGKGLEWVSGIGTGG | SCB-VH-88 |
| | ATSYADSVKGRFTISRDNAKNSLYLQMNSLRAEDTAVYYCARGRYYFPWWGQGTLV | |
| | TVSS | |
| 62 | EVQLVQSGGGLVHPGGSLRLSCAGSGFTFSRNAMFWVRQAPGKGLEWVSGIGTGG | SCB-VH-89 |
| | ATSYADSVKGRFTISRDNAKNSLYLQMNSLRAEDTAVYYCARGRYYFPWWGQGTLV | |
| | TVSS | |
| 63 | EVQLVESGGGLVQPGGSLRLSCAASGFTFSRNAMFWVRQAPGKGLEWVSGIGTGG | SCB-VH-90 |
| | ATNYADSVKGRFTISRDNAKNSLYLQMNSLRAEDMAVYYCARGRYYFPWWGQGTL | |
| | VTVSS | |
| 64 | EVQLVESGGGLVQPGGSLRLSCAASGFTFSRNAMFWVRQAPGKGLEWVSGIGTGG | SCB-VH-91 |
| | ATSYADSVKGRFTISRDNAKNSLYLQMNSLRAEDTAVYYCARGRYYFDYWGQGTLV | |
| | TVSS | |
| 65 | EVQLVQSGGGLVHPGGSLRLSCAGSGFTFSRNAMFWVRQAPGKGLEWVSGIGTGG | SCB-VH-92 |
| | ATNYADSVKGRFTISRDNAKNSLYLQMNSLRAEDMAVYYCARGRYYFDYWGQGTL | |
| | VTVSS | |
| 66 | EVQLVESGGGLVQPGGSLRLSCAASGFTFSRNAMFWVRQAPGKGLEWVSGIGTGG | SCB-VH-93 |
| | ATSYADSVKGRFTISRDNAKNSLYLQMNSLRAEDTAVYYCARGRYYFPWWGQGTLV | |
| | TVSS | |
| 67 | QVQLVQSGAEVKKPGASVKVSCKASGYAFTSYYMHWARQAPGQGLEWMGIINPS | MEDI-1-VH |
| | GGSTSYAQKFQGRVTMTRDTSTSTVYMELSSLRSEDTAVYYCARGKWWLDYWGKG | |
| | TLVTVSS | |
| 68 | QSVLTQPPSVSAAPGQKVTISCSGGSSNIGNSYVSWYQQLPGTAPKLLIYDNNKRPS | MEDI-1-VL |
| | GIPDRFSGSKSGTSATLAITGLQTGDEADYYCGTWDTSLSANYVFGTGTKLTVL | |
| 69 | QVQLVQSGAEVKKPGASVKVSCKASGYAFTSYYMHWARQAPGQGLEWMGIINPS | MEDI-2-VH |
| | GGSTSYAQKFQGRVTMTRDTSTSTVYMELSSLRSEDTAVYYCARGKWWLYNWGKG | |
| | TLVTVSS | |
| 70 | QSVLTQPPSVSAAPGQKVTISCSGGSSNIGNSYVSWYQQLPGTAPKLLIYDNNKRPS | MEDI-2-VL |
| | GIPDRFSGSKSGTSATLAITGLQTGDEADYYCGTWDTSQPPNPLFGTGTKLTVL | |
| 71 | QVQLVQSGAEVKKPGASVKVSCKASGYAFTSYYMHWARQAPGQGLEWMGIINPS | MEDI-3-VH |
| | GGSTSYAQKFQGRVTMTRDTSTSTVYMELSSLRSEDTAVYYCARGKLLKNPWGKGT | |
| | LVTVSS | |
| 72 | QSVLTQPPSVSAAPGQKVTISCSGGSSNIGNSYVSWYQQLPGTAPKLLIYDNNKRPS | MEDI-3-VL |
| | GIPDRFSGSKSGTSATLAITGLQTGDEADYYCGTWFGTPASNYVFGTGTKLTVL | |
| 73 | QVQLVQSGAEVKKPGASVKVSCKASGYAFTSYYMHWARQAPGQGLEWMGIINPS | MEDI-4-VH |
| | GGSTSYAQKFQGRVTMTRDTSTSTVYMELSSLRSEDTAVYYCARGKWWLYNWGKG | |
| | TLVTVSS | |
| 74 | QSVLTQPPSVSAAPGQKVTISCSGGSSNIGNSYVSWYQQLPGTAPKLLIYDNNKRPS | MEDI-4-VL |
| | GIPDRFSGSKSGTSATLAITGLQTGDEADYYCGTWDTSSPPQPIFGTGTKLTVL | |
| 75 | QVQLVQSGAEVKKPGASVKVSCKASGYAFTSYYMHWARQAPGQGLEWMGIINPS | MEDI-5-VH |
| | GGSTSYAQKFQGRVTMTRDTSTSTVYMELSSLRSEDTAVYYCARGKWWLYDWGKG | |
| | TLVTVSS | |
| 76 | QSVLTQPPSVSAAPGQKVTISCSGGSSNIGNSYVSWYQQLPGTAPKLLIYDNNKRPS | MEDI-5-VL |
| | GIPDRFSGSKSGTSATLAITGLQTGDEADYYCGTWDTSSPPQPIFGTGTKLTVL | |
| 77 | QVQLVQSGAEVKKPGASVKVSCKASGYAFTSYYMHWARQAPGQGLEWMGIINPS | MEDI-6-VH |
| | GGSTSYAQKFQGRVTMTRDTSTSTVYMELSSLRSEDTAVYYCARGKYWMYDWGKG | |
| | TLVTVSS | |
| 78 | QSVLTQPPSVSAAPGQKVTISCSGGSSNIGNSYVSWYQQLPGTAPKLLIYDNNKRPS | MEDI-6-VL |
| | GIPDRFSGSKSGTSATLAITGLQTGDEADYYCGTWDTSTTYHPIFGTGTKLTVL | |
| 79 | QVQLVQSGAEVKKPGASVKVSCKASGYAFTSYYMHWARQAPGQGLEWMGIINPS | MEDI-7-VH |
| | GGSTSYAQKFQGRVTMTRDTSTSTVYMELSSLRSEDTAVYYCARGKWWWQYWGK | |
| | GTLVTVSS | |
| 80 | QSVLTQPPSVSAAPGQKVTISCSGGSSNIGNSYVSWYQQLPGTAPKLLIYDNNKRPS | MEDI-7-VL |
| | GIPDRFSGSKSGTSATLAITGLQTGDEADYYCGTWDTSSPPQPIFGTGTKLTVL | |
| 81 | QVQLVQSGAEVKKPGASVKVSCKASGYAFTSYYMHWARQAPGQGLEWMGIINPS | MEDI-8-VH |
| | GGSTSYAQKFQGRVTMTRDTSTSTVYMELSSLRSEDTAVYYCARGKWWWQYWGK | |
| | GTLVTVSS | |
| 82 | QSVLTQPPSVSAAPGQKVTISCSGGSSNIGNSYVSWYQQLPGTAPKLLIYDNNKRPS | MEDI-8-VL |
| | GIPDRFSGSKSGTSATLAITGLQTGDEADYYCGTWDTSTTYHPIFGTGTKLTVL | |
| 83 | QVQLVQSGAEVKKPGASVKVSCKASGYAFTSYYMHWARQAPGQGLEWMGIINPS | MEDI-9-VH |
| | GGSTSYAQKFQGRVTMTRDTSTSTVYMELSSLRSEDTAVYYCARGKWWLYNWGKG | |
| | TLVTVSS | |
| 84 | QSVLTQPPSVSAAPGQKVTISCSGGSSNIGNSYVSWYQQLPGTAPKLLIYDNNKRPS | MEDI-9-VL |
| | GIPDRFSGSKSGTSATLAITGLQTGDEADYYCGTWDTSTTMYPLFGTGTKLTVL | |
| 85 | QVQLVQSGAEVKKPGASVKVSCKASGYAFTSYYMHWARQAPGQGLEWMGIINPS | MEDI-10-VH |
| | GGSTSYAQKFQGRVTMTRDTSTSTVYMELSSLRSEDTAVYYCARGKWWLYDWGKG | |
| | TLVTVSS | |
| 86 | QSVLTQPPSVSAAPGQKVTISCSGGSSNIGNSYVSWYQQLPGTAPKLLIYDNNKRPS | MEDI-10-VL |
| | GIPDRFSGSKSGTSATLAITGLQTGDEADYYCGTWDTSTVLTPIFGTGTKLTVL | |
| 87 | QVQLVQSGAEVKKPGASVKVSCKASGYAFTSYYMHWARQAPGQGLEWMGIINPS | MEDI-11-VH |
| | GGSTSYAQKFQGRVTMTRDTSTSTVYMELSSLRSEDTAVYYCARGKWWFYDWGKG | |
| | TLVTVSS | |
| 88 | QSVLTQPPSVSAAPGQKVTISCSGGSSNIGNSYVSWYQQLPGTAPKLLIYDNNKRPS | MEDI-11-VL |
| | GIPDRFSGSKSGTSATLAITGLQTGDEADYYCGTWDTSPSMIPLFGTGTKLTVL | |
| 89 | QVQLVQSGAEVKKPGASVKVSCKASGYAFTSYYMHWARQAPGQGLEWMGIINPS | MEDI-12-VH |
| | GGSTSYAQKFQGRVTMTRDTSTSTVYMELSSLRSEDTAVYYCARGKWWFYDWGKG | |
| | TLVTVSS | |
| 90 | QSVLTQPPSVSAAPGQKVTISCSGGSSNIGNSYVSWYQQLPGTAPKLLIYDNNKRPS | MEDI-12-VL |
| | GIPDRFSGSKSGTSATLAITGLQTGDEADYYCGTWDTSTTMYPLFGTGTKLTVL | |
| 91 | QVQLVQSGAEVKKPGASVKVSCKASGYAFTSYYMHWARQAPGQGLEWMGIINPS | MEDI-13-VH |
| | GGSTSYAQKFQGRVTMTRDTSTSTVYMELSSLRSEDTAVYYCARGKWWLYDWGKG | |
| | TLVTVSS | |
| 92 | QSVLTQPPSVSAAPGQKVTISCSGGSSNIGNSYVSWYQQLPGTAPKLLIYDNNKRPS | MEDI-13-VL |
| | GIPDRFSGSKSGTSATLAITGLQTGDEADYYCGTWDTSTTLQPLFGTGTKLTVL | |
| 93 | QVQLVQSGAEVKKPGASVKVSCKASGYAFTSYYMHWARQAPGQGLEWMGIINPS | MEDI-14-VH |
| | GGSTSYAQKFQGRVTMTRDTSTSTVYMELSSLRSEDTAVYYCARGKWWLYNWGKG | |
| | TLVTVSS | |
| 94 | QSVLTQPPSVSAAPGQKVTISCSGGSSNIGNSYVSWYQQLPGTAPKLLIYDNNKRPS | MEDI-14-VL |
| | GIPDRFSGSKSGTSATLAITGLQTGDEADYYCGTWDTSPPTKPLFGTGTKLTVL | |
| 95 | QVQLVQSGAEVKKPGASVKVSCKASGYAFTSYYMHWARQAPGQGLEWMGIINPS | MEDI-15-VH |
| | GGSTSYAQKFQGRVTMTRDTSTSTVYMELSSLRSEDTAVYYCARGKWWLYNWGKG | |
| | TLVTVSS | |
| 96 | QSVLTQPPSVSAAPGQKVTISCSGGSSNIGNSYVSWYQQLPGTAPKLLIYDNNKRPS | MEDI-15-VL |
| | GIPDRFSGSKSGTSATLAITGLQTGDEADYYCGTWDTSTHRHPLFGTGTKLTVL | |
| 97 | QVQLVQSGAEVKKPGASVKVSCKASGYAFTSYYMHWARQAPGQGLEWMGIINPS | MEDI-16-VH |
| | GGSTSYAQKFQGRVTMTRDTSTSTVYMELSSLRSEDTAVYYCARGKWWLYNWGKG | |
| | TLVTVSS | |
| 98 | QSVLTQPPSVSAAPGQKVTISCSGGSSNIGNSYVSWYQQLPGTAPKLLIYDNNKRPS | MEDI-16-VL |
| | GIPDRFSGSKSGTSATLAITGLQTGDEADYYCGTWDTSTTYHPIFGTGTKLTVL | |
| 99 | QVQLVQSGAEVKKPGASVKVSCKASGYAFTSYYMHWARQAPGQGLEWMGIINPS | MEDI-17-VH |
| | GGSTSYAQKFQGRVTMTRDTSTSTVYMELSSLRSEDTAVYYCARGKWWWQHWGK | |
| | GTLVTVSS | |
| 100 | QSVLTQPPSVSAAPGQKVTISCSGGSSNIGNSYVSWYQQLPGTAPKLLIYDNNKRPS | MEDI-17-VL |
| | GIPDRFSGSKSGTSATLAITGLQTGDEADYYCGTWDTSPVDRPIFGTGTKLTVL | |
| 101 | QVQLVQSGAEVKKPGASVKVSCKASGYAFTSYYMHWARQAPGQGLEWMGIINPS | MEDI-18-VH |
| | GGSTSYAQKFQGRVTMTRDTSTSTVYMELSSLRSEDTAVYYCARGKWWWQHWGK | |
| | GTLVTVSS | |
| 102 | QSVLTQPPSVSAAPGQKVTISCSGGSSNIGNSYVSWYQQLPGTAPKLLIYDNNKRPS | MEDI-18-VL |
| | GIPDRFSGSKSGTSATLAITGLQTGDEADYYCGTWDTSTTPMPVFGTGTKLTVL | |
| 103 | QVQLVQSGAEVKKPGASVKVSCKASGYAFTSYYMHWARQAPGQGLEWMGIINPS | MEDI-19-VH |
| | GGSTSYAQKFQGRVTMTRDTSTSTVYMELSSLRSEDTAVYYCARGKWWWQHWGK | |
| | GTLVTVSS | |
| 104 | QSVLTQPPSVSAAPGQKVTISCSGGSSNIGNSYVSWYQQLPGTAPKLLIYDNNKRPS | MEDI-19-VL |
| | GIPDRFSGSKSGTSATLAITGLQTGDEADYYCGTWDTSTTYHPIFGTGTKLTVL | |
| 105 | QVQLVQSGAEVKKPGASVKVSCKASGYAFTSYYMHWARQAPGQGLEWMGIINPS | MEDI-20-VH |
| | GGSTSYAQKFQGRVTMTRDTSTSTVYMELSSLRSEDTAVYYCARGKYWMYDWGKG | |
| | TLVTVSS | |
| 106 | QSVLTQPPSVSAAPGQKVTISCSGGSSNIGNSYVSWYQQLPGTAPKLLIYDNNKRPS | MEDI-20-VL |
| | GIPDRFSGSKSGTSATLAITGLQTGDEADYYCGTWDTSTVWEWPFGTGTKLTVL | |
| 107 | QVQLVQSGAEVKKPGASVKVSCKASGYAFTSYYMHWARQAPGQGLEWMGIINPS | MEDI-21-VH |
| | GGSASYAQKFQGRVTMTRDTSTSTVYMELSSLRSEDTAVYYCARGKYWMYDWGK | |
| | GTLVTVSS | |
| 108 | QSVLTQPPSVSAAPGQKVTISCSGGSSNIGNSYVSWYQQLPGTAPKLLIYDNNKRPS | MEDI-21-VL |
| | GIPDRFSGSKSGTSATLAITGLQTGDEAVYFCGTWDTSTVWEWPFGTGTKLTVL | |
| 109 | QVQLVQSGAEVKKPGASVKVSCKASGYAFTSYYMHWARQAPGQGLEWMGIINPS | MEDI-22-VH |
| | GGSTSYAQKFQGRVTMTRDTSTSTVYMELSSLRSEDTAVYYCARGKYWMYDWGKG | |
| | TLVTVSS | |
| 110 | QPVLTQPPSVSAAPGQKVTISCSGGSSNIGNSYVSWYQQLPGTAPKLLIYDNNKRPS | MEDI-22-VL |
| | GIPDRFSGSKSGTSATLAITGLQTGDEADYFCGTWDTSTVWEWPFGTGTKLTVL | |
| 111 | QVQLVQSGAEVRKPGASVKVSCKASGYAFTSYYMHWARQAPGQGLEWMGIINPS | MEDI-23-VH |
| | GGSTSYAQKFQGRVTMTRDTSTSTVYMELSSLRSEDTAVYYCARGKYWMYDWGKG | |
| | TLVTVSS | |
| 112 | QSVLTQPPSVSAAPGQKVTISCSGGSSNIGNNYVSWYQQLPGTAPKLLIYDNNKRPP | MEDI-23-VL |
| | GIPDRFSGSKSGTSATLAITGLQTGDEADYYCGTWDTSTVWEWPFGTGTKLTVL | |
| 113 | QVQLVQSGAEVKKPGASVKVSCKASGYAFTSYYMHWARQAPGQGLEWMGIINPR | MEDI-24-VH |
| | GGSTSYAQKFQGRVTMTRDTSTSTVYMELSSLRSEDTAVYYCARGKYWMYDWGKG | |
| | TLVTVSS | |
| 114 | QSVLTQPPSVSAAPGQKVTISCSGGSSNIGNSYVSWYQQLPGTAPKLLIYDNNKRPS | MEDI-24-VL |
| | GIPDRFSGSKSGTSATLAITGLQTGDEADYFCGTWDTSTVWEWPFGTGTKLTVL | |
| 115 | QVQLVQSGAEVKKPGASVKVSCKASGYAFTSYYMHWARQAPGQGLEWMGIINPR | MEDI-25-VH |
| | GGSASYAQKFQGRVSMTRDTSTSTVYMELSSLRSEDTAVYYCARGKYWMYDWGK | |
| | GTLVTVSS | |
| 116 | QSVLTQPPSVSAAPGQKVTISCSGGSSNIGNSYVSWYQQLPGTAPKLLIYDNNKRPS | MEDI-25-VL |
| | GIPDRFSGSKSGTTATLAITGLQTGDEADYYCGTWVTSTVWEWPFGTGTKLTVL | |
| 117 | QVQLVQSGAEVKKPGASVKVSCKASGYAFTSYYMHWARQAPGQGLEWMGIINPS | MEDI-26-VH |
| | GGSTSYAQKFQGRVTMTRDTSTSTVYMELSSLRSEDTAVYYCARGKYWMYDWGKG | |
| | TLVTVSS | |
| 118 | QSVLTQPPSVSAAPGQKVTISCSGGSSNIGNSYVSWYQQLPGTAPKLLIYDNNKRPS | MEDI-26-VL |
| | GIPDRFSGSKSGTSATLAITGLQTGDEADYFCGTWDTSTVWEWPFGTGTKLTVL | |
| 119 | QVQLVQSGAEVRKPGASVKVSCKASGYAFTSYYMHWARQAPGQGLEWMGIINPS | MEDI-27-VH |
| | GGSTSYAQKFQGRVTMTRDTSTSTVYMELSSLRPEDTAVYYCARGKYWMYDWGK | |
| | GTQVTVSS | |
| 120 | QSVLTQPPLVSAAPGQKVTISCSGGSSNIGNSYVSWYQRLPGTAPKLLIYDNNKRPSG | MEDI-27-VL |
| | IPDRFSGSKSGTSATLAITGLQTGDEADYYCGTWDTSTVWEWPFGTGTKLTVL | |
| 121 | QVQLVQSGAEVKKPGASVKVSCKASGYAFTSYYMHWARQAPGQGLEWMGIINPS | MEDI-28-VH |
| | GGSTSYAQKFQGRVTMTRDTSTSTVYMELSSLRSEDTAVYYCARGKYWMYDWGN | |
| | GTLVTVSS | |
| 122 | LPVLTQPPSVSAAPGQKVTISCSGGSSSIGNSYVSWYQQLPGAAPKLLIYDNNKRPSG | MEDI-28-VL |
| | IPDRFSGFRSGTSATLAITGLQTGDEADYYCGTWDTSPVWEWPFGTGTKLTVL | |
| 123 | QVQLVQSGAEVKKPGASVKVSCKASGYAFTSYYMHWARQAPGQGLEWMGIINPS | MEDI-29-VH |
| | GGSTSYAQKFQGRVTMTRDTSTSTVYMELSSLRSEDTAVYYCARGKYWMYDWGKG | |
| | TRVTVSS | |
| 124 | QSVLTQPPSVSAAPGQKVTISCSGGSSNIGNSYVSWYQQLPGTAPKLLIYDNNKRPS | MEDI-29-VL |
| | GIPDRFSGSKSGTSATLAITGLQTGDEADYYCGTWDTSPVWEWPFGTGTKLTVL | |
| 125 | QVQLVQSGAEVKKPGASVKVSCKASGYAFTSYYMHWARQAPGQGLEWMGIINPS | MEDI-30-VH |
| | GGSTSYAQKFQGRVTMTRDTSTSTVYMELSSLRSEDTAVYYCARGKYWMYDWGKG | |
| | TLVTVSS | |
| 126 | QSVLTQPPSVSAAPGQKVTISCSGGSSNIGNSYVSWYQRLPGAAPKLLIYDNNKRPS | MEDI-30-VL |
| | GIPDRFSGSKSGTSATLAITGLQTGDEADYYCGTWDTSTVWEWPFGTGTKLTVL | |
| 127 | QVQLVQSGAEVKKPGASVKVSCKASGYAFTSYYMHWARQAPGQGLEWMGIINPS | MEDI-31-VH |
| | GGSTSYAQKFQGRVTMTRDTSTSTVYMELSSLRSEDTAVYYCARGKYWMYDWGKG | |
| | TLVTVSS | |
| 128 | QSVLTQPPSVSAAPGQKVTISCSGGSSSIGNSYVSWYQQLPGTAPKLLIYDNNKRPSG | MEDI-31-VL |
| | IPDRFSGSKSGTSATLAITGLQTGDEADYYCGTWATSPVWEWPFGTGTKLTVL | |
| 129 | QVQLVQSGAEVKKPGASVKVSCKASGYAFTSYYMHWARQAPGQGLEWMGIINPS | MEDI-32-VH |
| | GGSTSYAQKFQGRVTMTRDTSTSTVYMELSSLRSEDTAVYYCARGKYWMYDWGKG | |
| | TLVTVSS | |
| 130 | QSVLTQPPSVSAAPGQKVTISCSGGSSNIGNSYVSWYQQLPGTAPKLLIYDNNKRPS | MEDI-32-VL |
| | GIPDRFSGSKSGTSATLAITGLQTGDEADYFCGTWDTSTAWEWPFGTGTKLTVL | |
| 131 | QVQLVQSGAEEKKPGASVKVSCKASGYAFTSYYMHWARQAPGQGLEWMGIINPS | MEDI-33-VH |
| | GGSTSYAQKFQGRVTMTRDTSTSTVYMELSSLRSEDTAVYYCARGKYWMYDWGKG | |
| | TLVTVSS | |
| 132 | QSALTQPPSVSAAPGQKVTISCSGGSSNIGNSYVSWYQQLPGTAPKLLIYDNNKRPS | MEDI-33-VL |
| | GIPDRFSGSKSGTSATLAITGLQTGDEADYFCGTWDTSTVWEWPFGTGTKLTVL | |
| 133 | QVQLVQSGAEVKKPGASVKVSCKASGYAFTSYYMHWARQAPGQGLEWMGIINPS | MEDI-34-VH |
| | GGSTSYAQKFQGRVSMTRDTSTSTVYMELSSLRSEDTAVYYCARGKYWMYDWGKG | |
| | TLVTVSS | |
| 134 | QSVLTQPPSVSAAPGQKVTISCSGGSSNIGNSYVSWYQQLPGTAPKLLIYDNNKRPS | MEDI-34-VL |
| | GIPDRFSGSKSGTSATLAITGLQTGDEADYFCGTWDTSTVWEWPFGTGTKLTVL | |
| 135 | QVQLVQSGAEVKKPGASVKVSCKASGYAFTSYYMHWARQAPGQGLEWMGIINPS | MEDI-35-VH |
| | GGSTSYAQKFQGRVTMTRDTSTSTVYMELSSLRSEDTAVYYCARGKYWMYDWGKG | |
| | TLVTVSS | |
| 136 | QSVLTQPPSVSAAPGQKVTISCSGGSSNIGNSYVSWYQQLPGTAPKLLIYDNNKRPS | MEDI-35-VL |
| | GIPDRFSGSKSGTSATLAITGLQTGDEADYYCGTWDTSPVWEWPFGTGTKLTVL | |
| 137 | QVQLVQSGAEVKKPGASVKVSCKASGYAFTSYYMHWARQAPGQGLEWMGIINPS | MEDI-36-VH |
| | GGSASYAQKFQGRVTMTRDTSTSTVYMELSSLRSEDTAVYYCARGKYWMYDWGK | |
| | GTLVTVSS | |
| 138 | QSVLTQPPSVSAAPGQKVTISCSGGSSNIGNSYVSWYQQLPGTAPKLLIYDNNKRPS | MEDI-36-VL |
| | GIPDRFSGSKSGTSATLAITGLQTGDEADYYCGTWDSSTVWEWPFGTGTKLTVL | |
| 139 | QVQLVQSGAEVKKPGASVKVSCKASGYAFTSYYMHWARQAPGQGLEWMGIINPR | MEDI-37-VH |
| | GGSTSYAQKFQGRVAMTRDTSTSTVYMELSSLRPEDTAVYYCARGKYWMYDWGK | |
| | GTLVTVSS | |
| 140 | QSVLTQPPSVSAAPGQKVTISCSGGGSSIGNSYVSWYQQLPGTAPKLLIYDNNKRPS | MEDI-37-VL |
| | GVPDRFSGSKSGTSATLAITGLQTGDEADYYCGTWDTSPVWEWPFGTGTKLTVL | |
| 141 | QVQLVQSGAEVKKPGASVKVSCKASGYAFTSYYMHWARQAPGQGLEWMGIINPS | MEDI-38-VH |
| | GGSASYAQKFQGRVTMTRDTSTSTVYMELSSLRSEDTAVYYCARGKYWMYDWGK | |
| | GTLVTVSS | |
| 142 | QSVLTQPPSVSAAPGQKVTISCSGGSSNIGNSYVSWYQQLPGTAPKLLIYDNNKRPS | MEDI-38-VL |
| | GIPDRFSGSKSGTSATLAITGLQTGDEADYFCGTWDTSTVWEWPFGTGTKLTVL | |
| 143 | QVQLVQSGAEVKKPGASVKVSCKASGYAFTSYYMHWARQAPGQGLEWMGIINPR | MEDI-39-VH |
| | GGSTSYAQKFQGRVTMTRDTSTSTVYMELSSLRSEDTAVYYCARGKYWMYDWGKG | |
| | TLVTVSS | |
| 144 | QSVLTQPPSVSAAPGQKVTISCSGGSSNIGNSYVSWYQQLPGTAPKLLIYDNNKRPS | MEDI-39-VL |
| | GIPDRFSGSKSGTSATLAITGLQTGDEADYYCGTWDTSTAWEWPFGTGTKLTVL | |
| 145 | QVQLVQSGAEVKKPGASVKVSCKASGYAFTSYYMHWARQAPGQGLEWMGIINPS | MEDI-40-VH |
| | GGSTSYAQKFQGRVTMTRDTSTSTVYMELSSLRSEDTAVYYCARGKYWMYDWGKG | |
| | TLVTVSS | |
| 146 | QSVLTQPPSVSAAPGQKVTISCSGGSSNIGNSYVSWYQQLPGTAPKLLIYDNNKRPS | MEDI-40-VL |
| | GIPDRFSGSKSGTSATLAITGLQTGDEADYYCGTWDSSTVWEWPFGTGTKLTVL | |
| 147 | QVQLVQSGAEVRKPGASVKVSCKASGYAFTSYYMHWARQAPGQGLEWMGIINPS | MEDI-41-VH |
| | GGSTSYAQKFQGRVTMTRDTSTSTVYMELSSLRPEDTAVYYCARGKYWMYDWGK | |
| | GTLVTVSG | |
| 148 | QSVLTQPPSVSAAPGQKVTISCSGGSTNIGNSYVSWYQRLPGTAPKLLIYDNNKRPP | MEDI-41-VL |
| | GIPDRFSGSKSGTSATLAITGLQTGDEADYYCGTWDTSTVWEWPFGTGTKLTVL | |
| 149 | QVQLVQSGAEVKKPGASVKVSCKASGYAFTSYYMHWARQAPGQGLEWVGIINPSG | MEDI-42-VH |
| | GSTSYAQKFQGRVTMTRDTSTSTVYMELSSLRSGDTAVYYCARGKYWMYDWGKGT | |
| | LVTVSS | |
| 150 | QAVLTQPPSVSAAPGQKVTISCSGGSSNIGNSYVSWYQRLPGAAPKLLIYDNNKRPS | MEDI-42-VL |
| | GIPDRFSGSKSGTSATLAITGLQTGDEADYYCGTWDTSTGWEWPFGTGTKLTVL | |
| 151 | QVQLVQSGAEVKKPGASVKVSCKASGYAFTSYYMHWVRQAPGQGLEWMGIINPR | MEDI-37GL- |
| | GGSTSYAQKFQGRVTMTRDTSTSTVYMELSSLRSEDTAVYYCARGKYWMYDWGKG | VH |
| | TLVTVSS | |
| 152 | QSVLTQPPSVSAAPGQKVTISCSGGGSSIGNSYVSWYQQLPGTAPKLLIYDNNKRPS | MEDI-37GL- |
| | GIPDRFSGSKSGTSATLGITGLQTGDEADYYCGTWDTSPVWEWPFGTGTKLTVL | VL |
| 153 | EVQLLESGGGLVQPGGSLRLSCAVSGFTFSNYAMSWVRQAPGKGLEWVSAISSGGG | AJOU-1-VH |
| | NIYYADSVKGRFTISRDNSKNTLYLQMNSLRAEDTAVYYCAKLRRYFDYWGQGTLVT | |
| | VSS | |
| 154 | EVQLLESGGGLVQPGGSLRLSCAASGFTFSDYAMSWVRQAPGKGLEWVSAISSGGS | AJOU-2-VH |
| | SIYYADSVKGRFTISRDNSKNTLHLQMNSLRAEDTAVYYCARGPQRSATAVFDYWG | |
| | QGTLVTVSS | |
| 155 | EVQLLESGGGLVQPGGSLRLSCAASGFTFSNYAMSWVRQAPGKGLEWVSWISPNS | AJOU-3-VH |
| | GNIYYADSVKGRFTISRDNSKNTLYLQMNSLRAEDTAVYYCARRPLSAAWSHSSYYN | |
| | AMDVWGQGTLVTVSS | |
| 156 | EVQLLESGGGLVQPGGSLRLSCAASGFTFSGYAMSWVRQAPGKGLEWVSLISHSGS | AJOU-4-VH |
| | NTYYADSVKGRFTISRDNSKNTLYLQMNSLRAEDTAVYYCARPHRAFDYWGQGTLV | |
| | TVSS | |
| 157 | EVQLLESGGGLVQPGGSLRLSCAASGFTFSNYAMSWVRQAPGKGLEWVSGISHGS | AJOU-5-VH |
| | GSIYYADSVKGRFTISRDNSKNTLYLQMNSLRAEDTAVYYCARPHRAFDYWGQGTLV | |
| | TVSS | |
| 158 | EVQLLESGGGLVQPGGSLRLSCAASGFTFSNYAMSWVRQAPGKGLEWVSGISHGN | AJOU-6-VH |
| | GSIYYADSVKGRFTISRDNSKNTLYLQMNSLRAEDTAVYYCAKTGRHFDYWGQGTLV | |
| | TVSS | |
| 159 | EVQLLESGGGLVQPGGSLRLSCAASGFTFSNYAMSWVRQAPGKGLEWVSSISPSGS | AJOU-7-VH |
| | SIYYADSVKGRFTISRDNSKNTLYLQMNSLRAEDTAVYYCARSYRAFDYWGQGTLVT | |
| | VSS | |
| 160 | EVQLLESGGGLVQPGGSLRLSCAASGFTFSNYAMSWVRQAPGKGLEWVSAISPSGG | AJOU-8-VH |
| | SIYYADSVKGRFTISRDNSKNTLYLQMNSLRAEDTAVYYCARAKRAFDYWGQGTLVT | |
| | VSS | |
| 161 | EVQLLESGGGLVQPGGSLRLSCAASGFTFSNYAMSWVRQAPGKGLEWVSAISPGSG | AJOU-9-VH |
| | STYYADSVKGRFTISRDNSKNTLYLQMNSLRAEDTAVYYCAKFRRHFDYWGQGTLVT | |
| | VSS | |
| 162 | EVQLLESGGGLVQPGGSLRLSCAASGFTFSNYAMSWVRQAPGKGLEWVSAISSGGG | AJOU-10-VH |
| | NIYYADSVKGRFTISRDNSKNTLYLQMNSLRAEDTAVYYCARVHRAFDYWGQGTLV | |
| | TVSS | |
| 163 | EVQLLESGGGLVQPGGSLRLSCAASGFTFSNYAMSWVRQAPGKGLEWVSAITSSGR | AJOU-69-VH |
| | SIYYADSVKGRFTISRDNSKNTLYLQMNSLRAEDTAVYYCARVHRAFDYWGQGTLVT | |
| | VSS | |
| 164 | EVQLLESGGGLVQPGGSLRLSCAASGFTFSNYAMSWVRQAPGKGLEWVSAITSSGA | AJOU-70-VH |
| | NIYYADSVKGRFTISRDNSKNTLYLQMNSLRAEDTAVYYCARVHRAFDYWGQGTLV | |
| | TVSS | |
| 165 | EVQLLESGGGLVQPGGSLRLSCAASGFTFSNYAMSWVRQAPGKGLEWVSAITSSGG | AJOU-71-VH |
| | NIYYADSVKGRFTISRDNSKNTLYLQMNSLRAEDTAVYYCARVHRAFDYWGQGTLV | |
| | TVSS | |
| 166 | EVQLLESGGGLVQPGGSLRLSCAASGFTFSNYAMSWVRQAPGKGLEWVSAITAGG | AJOU-72-VH |
| | GSIYYADSVKGRFTISRDNSKNTLYLQMNSLRAEDTAVYYCARVHRAFDYWGQGTLV | |
| | TVSS | |
| 167 | EVQLLESGGGLVQPGGSLRLSCAASGFTFSRHAMAWVRQAPGKGLEWVSAITSSGR | AJOU-83-VH |
| | SIYYADSVKGRFTISRDNSKNTLYLQMNSLRAEDTAVYYCARVHRAFDYWGQGTLVT | |
| | VSS | |
| 168 | QSVLTQPPSASGTPGQRVTISCSGSSSNIGNNYVNWYQQLPGTAPKLLIYDNSHRPS | AJOU-33-VL |
| | GVPDRFSGSKSGTSASLAISGLRSEDEADYYCGTWDASLSAYVFGGGTKLTVL | |
| 169 | QSVLTQPPSASGTPGQRVTISCSGSSSNIGNNNVSWYQQLPGTAPKLLIYANSKRPS | AJOU-34-VL |
| | GVPDRFSGSKSGTSASLAISGLRSEDEADYYCGSWDDSLSAYVFGGGTKLTVL | |
| 170 | QSVLTQPPSAPGTPGQRVTISCTGSSSNIGSNSVNWYQQLPGTAPKLLIYDDSHRPS | AJOU-35-VL |
| | GVPDRFSGSKSGTSASLAISGLRSEDEADYYCDAWDSSLSAYVFGGGTKLTVL | |
| 171 | QSVLTQPPSASGTPGQRVTLSCTGSSSNIGSNYVSWYQQLPGTAPKLLIYADSQRPSG | AJOU-36-VL |
| | VPDRFSGSKSGTSASLAISGLRSEDEADYYCGTWDDSLSGYVFGGGTKLTVL | |
| 172 | QSVLTQPPSASGTPGQRVTISCSSSSSNIGSNYVSWYQQLPGTAPKLLIYSDSHRPSG | AJOU-37-VL |
| | VPDRFSGSKSGTSASLAISGLRSEDEADYYCGSWDYSLSAYVFGGGTKLTVL | |
| 173 | QSVLTQPPSASGTPGQRVTISCTGSSSNIGNNTVSWYQQLPGTAPKLLIYDNSHRPS | AJOU-38-VL |
| | GVPDRFSGSKSGTSASLAISGLQSEDEADYYCGSWDYSLSAYVFGGGTKLTVL | |
| 174 | QSVLTQPPSASGTPGQRVTISCTGSSSNIGNNDVNWYQQLPGTAPKLLIYYDSQRPS | AJOU-39-VL |
| | GVPDRFSGSKSGTSASLAISGLRSEDEADYYCATWDASLSAYVFGGGTKLTVL | |
| 175 | QSVLTQPPSASGTPGQRVTISCSGSSSNIGSNAVNWYQQLPGTAPKLLIYYDNQRPS | AJOU-40-VL |
| | GVPDRFSGSKSGTSASLAISGLRSEDEADYYCGTWDDSLNGYVFGGGTKLTVL | |
| 176 | QSVLTQPPSASGTPGQRVTISCSGSSSNIGNNAVTWYQQLPGTAPKLLIYDDSHRPS | AJOU-41-VL |
| | GVPDRFSGSKSGTSASLAISGLRSEDEADYYCGSWDYSLSAYVFGGGTKLTVL | |
| 177 | QSVLTQPPSASGTPGQRVTISCSGSSSNIGSNTFNWYQQLPGTAPKLLIYADSHRPSG | AJOU-42-VL |
| | VPDRFSGSKSGTSASLAISGLRSEDEADYYCGTWDYSLSGYVLGGGTKLTVL | |
| 178 | QSVLTQPPSASGTPGQRVTISCSGSSSNIGSNTFNWYQQLPGTAPKLLIYADSHRPSG | AJOU-77-VL |
| | VPDRFSGSKSGTSASLAISGLRSEDEADYYCGTWDYSLSGYVLGGGTKLTVL | |
| 179 | QSVLTQPPSASGTPGQRVTISCSGSSSNIGSNTFNWYQQLPGTAPKLLIYADSHRPSG | AJOU-78-VL |
| | VPDRFSGSKSGTSASLAISGLRSEDEADYYCGTWDYSLRGYVLGGGTKLTVL | |
| 180 | QSVLTQPPSASGTPGQRVTISCSGSSSNIGSNTFNWYQQLPGTAPKLLIYADSHRPSG | AJOU-79-VL |
| | VPDRFSGSKSGTSASLAISGLRSEDEADYYCGYWDYSLSGYVLGGGTKLTVL | |
| 181 | QSVLTQPPSASGTPGQRVTISCSGSSSNIGSNTFNWYQQLPGTAPKLLIYADSHRPSG | AJOU-80-VL |
| | VPDRFSGSKSGTSASLAISGLRSEDEADYYCGTWDYSLSGYVLGGGTKLTVL | |
| 182 | QSVLTQPPSASGTPGQRVTISCSGSSANSRTDGFNWYQQLPGTAPKLLIYADSHRPS | AJOU-86-VL |
| | GVPDRFSGSKSGTSASLAISGLRSEDEADYYCGTWDYSLSGYVLGGGTKLTVLG | |
| 183 | QSVLTQPPSASGTPGQRVTISCSGSAQFGSRDNFNWYQQLPGTAPKLLIYADSHRPS | AJOU-87-VL |
| | GVPDRFSGSKSGTSASLAISGLRSEDEADYYCGTWDYSLSGYVLGGGTKLTVLG | |
| 184 | QSVLTQPPSASGTPGQRVTISCSGSTKQMHNYQFNWYQQLPGTAPKLLIYADSHRP | AJOU-88-VL |
| | SGVPDRFSGSKSGTSASLAISGLRSEDEADYYCGTWDYSLSGYVLGGGTKLTVLG | |
| 185 | QSVLTQPPSASGTPGQRVTISCSGSLLRGENLQFNWYQQLPGTAPKLLIYADSHRPS | AJOU-89-VL |
| | GVPDRFSGSKSGTSASLAISGLRSEDEADYYCGTWDYSLSGYVLGGGTKLTVLG | |
| 186 | QSVLTQPPSASGTPGQRVTISCSGSPLFPDSGSFNWYQQLPGTAPKLLIYADSHRPSG | AJOU-90-VL |
| | VPDRFSGSKSGTSASLAISGLRSEDEADYYCGTWDYSLSGYVLGGGTKLTVLG | |
| 187 | QSVLTQPPSASGTPGQRVTISCSGSAALDLSPSFNWYQQLPGTAPKLLIYADSHRPSG | AJOU-91-VL |
| | VPDRFSGSKSGTSASLAISGLRSEDEADYYCGTWDYSLSGYVLGGGTKLTVLG | |
| 188 | QVQLVQSGAEVKKPGASVKVSCKASGYTFTNYGISWVRQAPGQGLEWMGWISVY | REGN-VH-3 |
| | NGKTNYAQKLQGRVTMTTDTSTTTAYMEMRSLRSDDTAVYYCARGSGYDLDYWG | |
| | QGTLVSVSS | |
| 189 | EVQLVESGGGLVQPGGSLRLSCAASGFTFSSFWMTWVRQAPGKGLEWVANIKQD | REGN-VH-19 |
| | GSEKYYVDSVKGRFTISRDNAKNSLYLQMNSLRAEDTAVYYCARDPGRTMVRGGIRY | |
| | YYGMDVWGQGTTVTVSS | |
| 190 | EVKLAESGGGLVQPGGSLRLSCAASGFTFSSHWMNWVRQAPGKGLEWVANIKQD | REGN-VH-35 |
| | GSDKYYVDSVKGRFTISRDNAKNSLYLQLNSLIAEDTAVYYCARDRGVRPPRGAFDIW | |
| | GQGTMVTVSS | |
| 191 | QVQLVQSGAEVKKPGASVKVSCKASGYTFNSYGISWVRQAPGQGLEWMGWIRTY | REGN-VH-51 |
| | NGNTNYAQKLQGRVTMTTDTSTSTAYMELRSLRSDDTAVYYCARDEARIVVAGTTP | |
| | YYYGMDVWGQGTTVTVSS | |
| 192 | QVQLVESGGGLVQPGGSLRLSCAVSGFTISDHYMSWIRQAPGKGLEWISYISSSGSKI | REGN-VH-67 |
| | YYADSVKGRFTISRDNAKNSLFLQMNSLRAEDTAVYYCARTRQLVGDYWGQGTLVT | |
| | VSS | |
| 193 | EVQLVESGGGLVQPGRSLRLSCAASGFTFDNYAMHWVRQAPGKGLEWVSGIRWN | REGN-VH-83 |
| | SGSIGYADSVKGRFTISRDNAKNSLYLQMNSLRAEDTALYYCAKEGGYSGYRPGPFFD | |
| | YWGQGTLVTVSS | |
| 194 | QVQLVQSGAEVKKPGASVKVSCKASGYTFTNYGISWVRQAPGQGLEWMGWISVY | REGN-VH-99 |
| | NGHTNYAQKLQGRVTMTTDTSTSTAYMELRSLRSDDTAVYYCARGSGYDFDSWGQ | |
| | GTLVTVSS | |
| 195 | QVQLVQSGAEVKKPGASVKVSCKASRYTFTSYDINWVRQATGQGLEWMGWMNP | REGN-VH-115 |
| | NSGNTGYAQKFQGRVTMTRNTSTSTAYMELSSLRSEDTAVYYCARVRRFFDYWGQ | |
| | GTLVTVSS | |
| 196 | QVQLVQSGPEVKKPGASVKVSCKASGYTFTNYGISWVRQAPGQGLEWMGWISVY | REGN-VH-147 |
| | NGNINYAQKLQGRVTMTTDTSTSTAYMDLRSLRSDDTAVYYCARGSGYDFDYWGQ | |
| | GTLVTVSS | |
| 197 | QVQLVQSGAEVKKPGASVKVSCKDSAYTFNRYGISWVRQAPGQGLEWMGWISAY | REGN-VH-163 |
| | TGNTVYAQKLQGRVTMTTDNSTSTAYMELRSLRSDDTAVYYCARDKSIFGVVRGFD | |
| | YWGQGTLVTVSS | |
| 198 | AIQMTQSPSSLSASVGDRVTITCRASQGIRNALGWYQQKPGKAPKLLIYAASSLQSG | REGN-VL-11 |
| | VPSRFSGSGSGTDFTLTFSSLQPEDFATYYCLQDFNYPYTFGQGTKLEIK | |
| 199 | DIQMTQSPSSVSASVGDRVTISCRASQGVSSWLAWYQQKPGNAPKLLISAASSIQSG | REGN-VL-27 |
| | VPSRFSGSGSGTDFTLTISSLQPEDFATYYCQQANSFPLTFGGGTKVEIK | |
| 200 | DIQMTQSPSSVSASVGDRVTITCRASQGISSWLAWYQQKPGKAPKLLIYAASSFQSG | REGN-VL-43 |
| | VPSRFSGSGSGTDFTLTISSLQPEDFATYFCQQANSFPLTFGGGTTVEIK | |
| 201 | DIQMTQSPSSVSASVGDRVTITCRASQDISIWLAWYQQSPGKAPKLLINVASRLQSG | REGN-VL-59 |
| | VPSRFSGSGSGTDFTLTINSLQPEDFVTYYCQQANSFPITFGQGTRLATK | |
| 202 | DIQLTQSPSFLSASVGDRVTITCWASQGISSYLAWYQQKPGKAPKLLIFAASTLQSGV | REGN-VL-75 |
| | PSRFSGSGSGTEFTLTISSLQPEDFATYYCQQLNSYPLTFGGGTKVEIR | |
| 203 | EIVMTQSPATLSVSPGERATLSCRASQSVNYNLAWYQHKPGQAPRLLIYGASTRATGI | REGN-VL-91 |
| | PARFSGSGSGTEFTLTISSLQSEDFAVYYCQQYNNWPLTFGGGTKVEIK | |
| 204 | AIQMTQSSSSLSASVGDRVTITCRASQAIRNALGWYQQKPGKAPKVLIYAASSLQSGI | REGN-VL-107 |
| | PSRFSGSGSGTDFTLTISSLQPEDFATYYCLQDYDYPYTFGQGTKLEIK | |
| 205 | DIQLTQSPSFLSASVGDRVTITCWASQGIISYLAWYQQKPGKAPKLLIYAASTLHSGVP | REGN-VL-123 |
| | SRFSGSGSGTEFTLTISSLQPEDFATYYCHQLKSYPITFGQGTRLEIK | |
| 206 | AIQMTQSPSSLSASVGDRVTITCRASQDIRNALGWYQQKPGKAPKLLIYAASSLQSG | REGN-VL-155 |
| | VPSRFSGSASGTDFTLTISSLQPEDFAAYYCLQDYNYPYTFGQGTKLEIK | |
| 207 | EIVMTQSPVTLSLSPGERATLPCRASQSVSSSLAWYQQKAGQSPRLLIYGASTRATGI | REGN-VL-171 |
| | PARFSGSGSGTEFTLTISNLQSEDFAVYYCQQYNNWPLTFGGGTKVEIK | |
| 208 | EVQLLESGGGLVQPGGSLRLSCAASGFTLSSYAMHWVRQAPGKGLEYVSGISSNGG | STSA-C27-VH |
| | STYYANSVKGRFTISRDNPKNTLFLQMSSLRAEDTAVYYCVRVKVGYRGGMDVWG | |
| | QGTTVTVSS | |
| 209 | EVQLLESGGGLVQPGGSLRLSCAASGFTLSSYAMHWVRQAPGKGLEYVSGISPSGSS | STSA-C27- |
| | TYYANSVKGRFTISRDNPKNTLFLQMSSLRAEDTAVYYCVRSKVRYRGGMDVWGQ | 6-33-VH |
| | GTTVTVSS | |
| 210 | EVQLLESGGGLVQPGGSLRLSCAASGFTLSSYAMHWVRQAPGKGLEYVSGISPSGVS | STSA-C27- |
| | TYYANSVKGRFTISRDNPKNTLFLQMSSLRAEDTAVYYCVRVKVKYRGGMDVWGQ | 7-33-VH |
| | GTTVTVSS | |
| 211 | EVQLLESGGGLVQPGGSLRLSCAASGFTLSSYAMHWVRQAPGKGLEYVSGISPTSGS | STSA-C27- |
| | TYYANSVKGRFTISRDNPKNTLFLQMSSLRAEDTAVYYCVRVKVRYRGGMDVWGQ | 24-56-VH |
| | GTTVTVSS | |
| 212 | EVQLLESGGGLVQPGGSLRLSCAASGFTLSSYAMHWVRQAPGKGLEYVSGISPTGTS | STSA-C27- |
| | TYYANSVKGRFTISRDNPKNTLFLQMSSLRAEDTAVYYCVRVKGAYRGGMDVWGQ | 47-56-VH |
| | GTTVTVSS | |
| 213 | EVQLLESGGGLVQPGGSLRLSCAASGFTLSSYAMHWVRQAPGKGLEYVSGISSSGSS | STSA-C27- |
| | TYYANSVKGRFTISRDNPKNTLFLQMSSLRAEDTAVYYCVRVKVAYRGGMDVWGQ | 33-33-VH |
| | GTTVTVSS | |
| 214 | EVQLLESGGGLVQPGGSLRLSCAASGFTLSSYAMHWVRQAPGKGLEYVSGISPSSTS | STSA-C27- |
| | TYYANSVKGRFTISRDNPKNTLFLQMSSLRAEDTAVYYCVRVKVLYRGGMDVWGQ | 56-56-VH |
| | GTTVTVSS | |
| 215 | EVQLLESGGGLVQPGGSLRLSCAASGFTLSSYAMHWVRQAPGKGLEYVSGISPSSAS | STSA-C27- |
| | TYYANSVKGRFTISRDNPKNTLFLQMSSLRAEDTAVYYCVRVKSKYRGGMDVWGQ | 78-78-VH |
| | GTTVTVSS | |
| 216 | EVQLLESGGGLVQPGGSLRLSCAASGFTLSSYAMHWVRQAPGKGLEYVSGISGNSAS | STSA-C27- |
| | TYYANSVKGRFTISRDNPKNTLFLQMSSLRAEDTAVYYCVRVKLKYRGGMDVWGQG | 82-58-VH |
| | TTVTVSS | |
| 217 | EVQLLESGGGLVQPGGSLRLSCAASGFTLSSYAMHWVRQAPGKGLEYVSGISHSGTS | STSA-C27- |
| | TYYANSVKGRFTISRDNPKNTLFLQMSSLRAEDTAVYYCVRVRVLYRGGMDVWGQ | 54-54-VH |
| | GTTVTVSS | |
| 218 | EVQLLESGGGLVQPGGSLRLSCAASGFTLSSYAMHWVRQAPGKGLEYVSGISPSGVS | STSA-C27- |
| | TYYANSVKGRFTISRDNPKNTLFLQMSSLRAEDTAVYYCVRVKVKYRGGMDVWGQ | 36-36-VH |
| | GTTVTVSS | |
| 219 | EVQLLESGGGLVQPGGSLRLSCAASGFTLSSYAMHWVRQAPGKGLEYVSGISSNGG | STSA-C27- |
| | STYYANSVKGRFTISRDNPKNTLFLQMSSLRAEDTAVYYCVRVFVRYRGGMDVWGQ | 53-53-VH |
| | GTTVTVSS | |
| 220 | EVQLLESGGGLVQPGGSLRLSCAASGFTLSSYAMHWVRQAPGKGLEYVSGISPTSAS | STSA-C27- |
| | TYYANSVKGRFTISRDNPKNTLFLQMSSLRAEDTAVYYCVRVKGRYRGGMDVWGQ | 67-67-VH |
| | GTTVTVSS | |
| 221 | EVQLLESGGGLVQPGGSLRLSCAASGFTLSSYAMHWVRQAPGKGLEYVSGISPTGGS | STSA-C27- |
| | TYYANSVKGRFTISRDNPKNTLFLQMSSLRAEDTAVYYCVRVKGRYRGGMDVWGQ | 55-55-VH |
| | GTTVTVSS | |
| 222 | EVQLLESGGGLVQPGGSLRLSCAASGFTLSSYAMHWVRQAPGKGLEYVSGISHSGN | STSA-C27- |
| | STYYANSVKGRFTISRDNPKNTLFLQMSSLRAEDTAVYYCVRVKRRYRGGMDVWGQ | 59-59-VH |
| | GTTVTVSS | |
| 223 | EVQLLESGGGLVQPGGSLRLSCAASGFTLSSYAMHWVRQAPGKGLEYVSGISPSSNS | STSA-C27- |
| | TYYANSVKGRFTISRDNPKNTLFLQMSSLRAEDTAVYYCVRVKVRYRGGMDVWGQ | 58-58-VH |
| | GTTVTVSS | |
| 224 | EVQLLESGGGLVQPGGSLRLSCAASGFTLSSYAMHWVRQAPGKGLEYVSGISSSGSS | STSA-C27- |
| | TYYANSVKGRFTISRDNPKNTLFLQMSSLRAEDTAVYYCVRVKPAYRGGMDVWGQ | 52-52-VH |
| | GTTVTVSS | |
| 225 | EVQLLESGGGLVQPGGSLRLSCAASGFTLSSYAMHWVRQAPGKGLEYVSGISYSSAS | STSA-C27- |
| | TYYANSVKGRFTISRDNPKNTLFLQMSSLRAEDTAVYYCVRVKVRYRGGMDVWGQ | Y2-Y2-VH |
| | GTTVTVSS | |
| 226 | ETTLTQSPDTLPLSPGDRASLSCRASQSVSSAYLAWYQQKPGQAPRLLIYGTSRRATG | STSA-C27- |
| | VPGRFSGSGSGTDFTLTISRLEPEDFAVYYCQLYGSSSVTFGQGTKLEIK | VL |
| 227 | EIVLTQSPGTLSLSPGERATLSCRASQGISSAYLAWYQQKPGQAPRLLIYGTSRRATGI | STSA-C27- |
| | PDRFSGSGSGTDFTLTISRLEPEDFAVYYCQLYGATSVTFGQGTKLEIK | 6-33-VL |
| 228 | EIVLTQSPGTLSLSPGERATLSCRASQGISSAYLAWYQQKPGQAPRLLIYGTSRRATGI | STSA-C27- |
| | PDRFSGSGSGTDFTLTISRLEPEDFAVYYCQLYGATSVTFGQGTKLEIK | 7-33-VL |
| 229 | EIVLTQSPGTLSLSPGERATLSCRASQSVSSAYLAWYQQKPGQAPRLLIYGTSRRATGI | STSA-C27- |
| | PDRFSGSGSGTDFTLTISRLEPEDFAVYYCQLYGASSVTFGQGTKLEIK | 24-56-VL |
| 230 | EIVLTQSPGTLSLSPGERATLSCRASQSVSSAYLAWYQQKPGQAPRLLIYGTSRRATGI | STSA-C27- |
| | PDRFSGSGSGTDFTLTISRLEPEDFAVYYCQLYGASSVTFGQGTKLEIK | 47-56-VL |
| 231 | EIVLTQSPGTLSLSPGERATLSCRASQGISSAYLAWYQQKPGQAPRLLIYGTSRRATGI | STSA-C27- |
| | PDRFSGSGSGTDFTLTISRLEPEDFAVYYCQLYGATSVTFGQGTKLEIK | 33-33-VL |
| 232 | EIVLTQSPGTLSLSPGERATLSCRASQSVSSAYLAWYQQKPGQAPRLLIYGTSRRATGI | STSA-C27- |
| | PDRFSGSGSGTDFTLTISRLEPEDFAVYYCQLYGASSVTFGQGTKLEIK | 56-56-VL |
| 233 | EIVLTQSPGTLSLSPGERATLSCRASQSISTAYLAWYQQKPGQAPRLLIYGTSRRATGIP | STSA-C27- |
| | DRFSGSGSGTDFTLTISRLEPEDFAVYYCQLYGASSVTFGQGTKLEIK | 78-78-VL |
| 234 | EIVLTQSPGTLSLSPGERATLSCRASQDISSAYLAWYQQKPGQAPRLLIYGTSRRATGI | STSA-C27- |
| | PDRFSGSGSGTDFTLTISRLEPEDFAVYYCQLYGATSVTFGQGTKLEIK | 82-58-VL |
| 235 | EIVLTQSPGTLSLSPGERATLSCRASQDVSSAYLAWYQQKPGQAPRLLIYGTSRRATGI | STSA-C27- |
| | PDRFSGSGSGTDFTLTISRLEPEDFAVYYCQLYGATSVTFGQGTKLEIK | 54-54-VL |
| 236 | EIVLTQSPGTLSLSPGERATLSCRASQNISTAYLAWYQQKPGQAPRLLIYGTSRRATGI | STSA-C27- |
| | PDRFSGSGSGTDFTLTISRLEPEDFAVYYCQLYGATSVTFGQGTKLEIK | 36-36-VL |
| 237 | EIVLTQSPGTLSLSPGERATLSCRASQDASNAYLAWYQQKPGQAPRLLIYGTSRRATG | STSA-C27- |
| | IPDRFSGSGSGTDFTLTISRLEPEDFAVYYCQLYGSSSVTFGQGTKLEIK | 53-53-VL |
| 238 | EIVLTQSPGTLSLSPGERATLSCRASQGVSSAYLAWYQQKPGQAPRLLIYGTSRRATGI | STSA-C27- |
| | PDRFSGSGSGTDFTLTISRLEPEDFAVYYCQLYGRSSVTFGQGTKLEIK | 67-67-VL |
| 239 | EIVLTQSPGTLSLSPGERATLSCRASQNISTAYLAWYQQKPGQAPRLLIYGTSRRATGI | STSA-C27- |
| | PDRFSGSGSGTDFTLTISRLEPEDFAVYYCQLYGTSSVTFGQGTKLEIK | 55-55-VL |
| 240 | EIVLTQSPGTLSLSPGERATLSCRASQSVSTAYLAWYQQKPGQAPRLLIYGTSRRATGI | STSA-C27- |
| | PDRFSGSGSGTDFTLTISRLEPEDFAVYYCQLYGATSVTFGQGTKLEIK | 59-59-VL |
| 241 | EIVLTQSPGTLSLSPGERATLSCRASQDISSAYLAWYQQKPGQAPRLLIYGTSRRATGI | STSA-C27- |
| | PDRFSGSGSGTDFTLTISRLEPEDFAVYYCQLYGATSVTFGQGTKLEIK | 58-58-VL |
| 242 | EIVLTQSPGTLSLSPGERATLSCRASQGVSTAYLAWYQQKPGQAPRLLIYGTSRRATGI | STSA-C27- |
| | PDRFSGSGSGTDFTLTISRLEPEDFAVYYCQLYGATSVTFGQGTKLEIK | 52-52-VL |
| 243 | EIVLPQSPGTLSLSPGERATLSCRASQGVSSAYLAWYQQKPGQAPRLLIYGTSRRATGI | STSA-C27- |
| | PDRFSGSGSGTDFTLTISRLEPEDFAVYYCQLYGSTSVTFGQGTKLEIK | Y2-Y2-VL |
| 244 | EVQLVESGGGLVQPKGSLKLSCAASGFTFNTYGMHWVRQAPGKGLEWVAHIRSKS | Y0188-1 VH |
| | SNYATYYADSVKDRFTISRDDSQSMLYLQMNNLKTEDTAMYYCVRWFRAMDYWG | |
| | QGTSVTVSS | |
| 245 | EVQLIESGGGLVQPKGSLKLSCAASGFTFNMYAMDWVRQAPGKGLEWVARIRSKG | Y0188-2 VH |
| | SNFETNYADSVKDRFTISRDDSQSMVYLQMINLKTEDTAMYYCVRHRGGAWFAYW | |
| | GQGTLVSVSA | |
| 246 | QVQLVETGGGLVRPGNSLKLSCVTSGFTFSNYRMHWLRQPPGKRLEWIAVITVKSN | Y0188-3 VH |
| | NYGANYAESVKGRFAISRDDSKSSVYLEMNRLREEDTATYFCSRERAYGNPFDYWG | |
| | QGTTLTVSS | |
| 247 | EVQLVESGGGLVQPKGSLKLSCAASGFTFNMYAMNWVRQAPGQGLEWVARIRSKS | Y0188-4 VH |
| | NNYATYYADSVKDRFIISRDDSESMVYLQMSNLRAADTAMYYCVRHLRAMDYWG | |
| | QGTSVTVSS | |
| 248 | EVQLVESGGGLVQPKGSLKLSCAASGFSFNMYAMNWVRQAPGKGLEWVARIRTKS | Y0188-6 VH |
| | NHYSTYYADSVKDRFTISRDDSASMFYLQMNNLKTEDTAMYFCVRHLRAMDYWG | |
| | QGTSVTVSS | |
| 249 | EVQLIESGGGLVQPKGSLKLSCAASGFTFNMYAMDWVRQAPGKGLEWVARIRSKG | Y0188-8 VH |
| | SNFETNYADSVKDRFTISRDDSQSMVYLQMNNLKTEDTAMYYCVRHRGGAWFAY | |
| | WGQGTLVTVSA | |
| 250 | EVQLVESGGGLVRPKGSLKLSCAASGFSFNTYAMNWVRQAPGKGLEWIVWIRSKSH | Y0188-9 VH |
| | NYATYYADSVKDRFTISRDDSESMLYLQMNNLKTEDTAMYYCVRHLRAMDYWGQ | |
| | GTSVTVSS | |
| 251 | EVRLVESGGGLVQPKGSLKLSCEASGFSFNMYAMNWVRQAPGKGLEWITHIRSKSN | Y0188-10 VH |
| | NYATYYADSVKDRFIISRDDSESMVYLQMNNLKTEDTAMYYCVRLLRALDYWGQGT | |
| | SVTVSS | |
| 252 | EVQLVESGGGLVQPKGSLKLSCAASGFTFNMYGMHWVRQAPGKGLEWVAHIRSKS | Y0188-14 VH |
| | SNYATYYADSVKDRLTISRDDSQSMLYLQMNNLKTEDTAMYYCVRWFRAMDYWG | |
| | QGTSVTVSS | |
| 253 | EVQLVESGGGLVKPGGSLRLSCAASGFTFSMYGMHWVRQAPGKGLEWVGHIRSKS | HV3-15- |
| | SNYATYYADSVKDRFTISRDDSKNTLYLQMNSLKTEDTAVYYCTTWFRAMDYWGQ | 14 VH |
| | GTLVTVSS | |
| 254 | EVQLVESGGGLVQPGGSLRLSCAASGFTFSMYGMHWVRQAPGKGLEWVSHIRSKS | HV3-48- |
| | SNYATYYADSVKDRFTISRDNAKNSLYLQMNSLRAEDTAVYYCARWFRAMDYWGQ | 14 VH |
| | GTLVTVSS | |
| 255 | EVQLVESGGGLVQPGGSLKLSCAASGFTFSMYGMHWVRQASGKGLEWVGHIRSKS | HV3-73*2- |
| | SNYATYYADSVKDRFTISRDDSKNTAYLQMNSLKTEDTAVYYCTRWFRAMDYWGQ | 14 VH |
| | GTLVTVSS | |
| 256 | EVQLVESGGGLVQPGGSLRLSCAASGFTFSMYGMHWVRQAPGKGLEWVGHIRSKS | HV3-72- |
| | SNYATYYADSVKDRFTISRDDSKNSLYLQMNSLKTEDTAVYYCARWFRAMDYWGQ | 14 VH |
| | GTLVTVSS | |
| 257 | EVQLVESGGGLVQPGGSLRLSCAASGFTFSMYGMHWVRQAPGKGLEWVSHIRSKS | Y01-14 VH |
| | SNYATYYADSVKDRFTISRDNAKNSLYLQMNSLRAEDTAVYYCARWFRAMDYWGQ | |
| | GTLVTVSS | |
| 258 | EVQLVESGGGLEQPGGSLRLSCAGSGFTFRMYGMHWVRQAPGKGLEWVSHIRSKS | 162-14 VH |
| | SNYATYYADSVKDRFTISRDNSKNTLYLQMNSLRAEDTAVYYCAKWFRAMDYWGQ | |
| | GTTVTVSS | |
| 259 | EVQLVESGGGLVQPGGSLKLSCAASGFTFSMYGMHWVRQASGKGLEWVGHIRSKS | VH73-14 VH |
| | SNYATYYADSVKDRFTISRDDSKNTAYLQMNSLKTEDTAVYYCTRWFRAMDYWGQ | |
| | GTTVTVSS | |
| 260 | DIVMTQSHKFMSTSVGDRVSITCKASQDVSTAVAWYQEKPGQSPKLLIYWASTRHT | Y0188-1 VL |
| | GVPDRFTGSGSGTDYTLTISSVQAEDLALYYCQQHYSTPLTFGAGTKLELK | |
| 261 | DIVVTQSPASLAVSLGQRATISCRASKSVSTSGYSYMHWYQQKPGQPPKLLIYLASNL | Y0188-2 VL |
| | ESGVPARFSGSGSGTDFTLNIHPVEEEDVAIYYCQHSRELPLTFGAGTKLELK | |
| 262 | DIQMTQSPSSLSASLGERVSLTCRASQEISGYLSWLQQKPDGTIKRLIYAASTLDSGVP | Y0188-3 VL |
| | KRFSGSRSGSDYSLTISSLESEDFADYYCLQYGSYPYTFGGGTKLEIK | |
| 263 | DIVLTQSPASLTVSLGQRATISCRASKSVSTSGYSYMHWYQQKPGQPPKLLIYLASNLE | Y0188-4 VL |
| | SGVPARFSGSGSGTDFTLNIHPVEEEDAATYYCQHSRELPITFGSGTKLEIK | |
| 264 | DIVLTQSPASLVVSLGQRATISCRASQSVSTSGYSYMHWYQQKPGQPPKLLIYLASNV | Y0188-6 VL |
| | QSGVPARFSGSGSGTDFTLNIHPVEEEDVATYYCHHNRDLPFTFGSGTKLEIK | |
| 265 | DIVVTQSPASLAVSLGQRATISCRASKSVSTSGYSYMHWYQQKPGQPPKLLIYLASNL | Y0188-8 VL |
| | ESGVPARFSGSGSGTDFTLNIHPVEEEDVAIYYCQHSRELPLTFGAGTKLELK | |
| 266 | DIVLTQSPASLAVSLGQRATISCRASKSVSASGYSYMHWYQQKPGQPPKLLIYLASNL | Y0188-9 VL |
| | QSGVPARFSGSGSGTDFTLNIHPVEEEDAATYYCQHSRELPPTFGGGTKLEIK | |
| 267 | DIVLTQSPASLAVFLGQRATISCRASKSVSTSGYSYMHWYQQKAGQPPKLLIYLASNL | Y0188-10 VL |
| | ESGVPARFSGSGSGTDFTLNIHPVEEEDAATYYCHHSRELPITFGSGTKLEMK | |
| 268 | DIVMTQSHKFMSTSVGDRVSITCKASQDVSTAVAWYQEKPGQSPKLLIYWASTRHT | Y0188-14 VL |
| | GVPDRFTGSGSGTDYTLTISSVQAEDLALYYCQQHYSTPLTFGAGTKLELK | |
| 269 | EIVLTQSPGTLSLSPGERATLSCKASQDVSTAVAWYQQKPGQAPRLLIYWASTRHTGI | Y01-14 VL |
| | PDRFSGSGSGTDFTLTISRLEPEDFAVYYCQQHYSTPLTFGQGTKVEIK | |
| 270 | DIVMTQSPLSLPVTPGEPASISCKASQDVSTAVAWYLQKSGQSPQLLIYWASTRHTG | 164-14 VL |
| | VPDRFSGSGSGTDFTLKISRVEAEDVGFYYCQQHYSTPLTFGQGTKLEIK | |
| 271 | DIVMTQSPDSLAVSLGERATINCKASQDVSTAVAWYQQKPGQPPKLLIYWASTRHT | KV4-14 VL |
| | GVPDRFSGSGSGTDFTLTISSLQAEDVAVYYCQQHYSTPLTFGGGTKVEIK | |
| 272 | DIQMTQSPSSLSASVGDRVTITCKASQDVSTAVAWYQQKPGKVPKLLIYWASTRHT | KV1-27- |
| | GVPSRFSGSGSGTDFTLTISSLQPEDVATYYCQQHYSTPLTFGGGTKVEIK | 14 VL |
| 273 | DIQLTQSPSFLSASVGDRVTITCKASQDVSTAVAWYQQKPGKAPKLLIYWASTRHTG | KV1-9- |
| | VPSRFSGSGSGTEFTLTISSLQPEDFATYYCQQHYSTPLTFGGGTKVEIK | 14 VL |
| 274 | DIQMTQSPSSLSASVGDRVTITCKASQDVSTAVAWYQQKPGKAPKLLLYWASTRHT | KV1-NL1- |
| | GVPSRFSGSGSGTDYTLTISSLQPEDFATYYCQQHYSTPLTFGGGTKVEIK | 14 VL |
| 275 | AIRMTQSPFSLSASVGDRVTITCKASQDVSTAVAWYQQKPAKAPKLFIYWASTRHTG | KV1D-43- |
| | VPSRFSGSGSGTDYTLTISSLQPEDFATYYCQQHYSTPLTFGGGTKVEIK | 14 VL |

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Prevention of Particle Formation in Lipase-Containing Formulations with PEG3350 or Poloxamer 188

Without intending to be bound by theory, it is believed that a putative phospholipase B-like 2 (PLBL2), which is highly conserved in hamster, rat, mice, human and bovine, copurifies under certain processes with some classes of proteins-of-interest produced in certain cell lines, and has esterase activity responsible for the hydrolysis of polysorbate 20 and 80. As an alternative to removing lipases through purification methods (e.g., as described in U.S. Pat. No. 10,342,876), formulations of IL-4R antibody were prepared using alternative surfactants, and particulate formation was measured over time. An anti-IL-4R antibody comprising the HCVR/LCVR amino acid sequence pair of SEQ ID NOs:1/2 at a concentration of 150 mg/mL of mAb1 was formulated with 20 mM histidine, 12.5 mM sodium acetate, 25 mM arginine-HCl, 5% w/v sucrose, and either PEG3350 or poloxamer 188 at varying concentrations, at pH 5.9. The formulations were stored in syringes at 5° C. for up to 36 months with periodic measurements taken of the number of particles (≥10 μm and ≥25 μm) present in the formulations, as determined by microscopy.

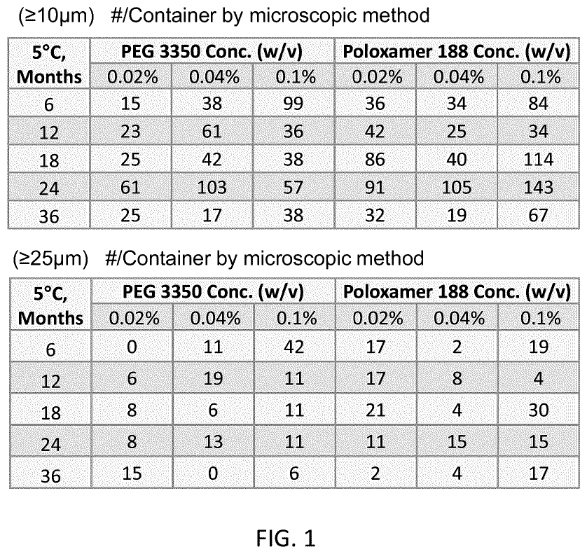

As shown in FIG. 1, few particles of ≥10 μm or ≥25 μm were identified in the formulations over the 36 month observation period. Moreover, no appreciable differences or changes in the number of subvisible particles among the different PEG3350- or poloxamer 188-containing formulations were observed over the course of the storage period. This experiment demonstrates that low concentrations of PEG3350 or poloxamer 188 can prevent particle formulation over long storage periods.

### Example 2: Agitation Stress Stability of Formulations Containing PEG3350 or Poloxamer 188

The agitation stress stability was tested for IL-4R antibody formulations containing different concentrations of the surfactant PEG3350 or poloxamer 188. An anti-IL-4R antibody comprising the HCVR/LCVR amino acid sequence pair of SEQ ID NOs:1/2 at a concentration of 150 mg/mL was formulated with 20 mM histidine, 12.5 mM sodium acetate, 25 mM arginine-HCl, 5% w/v sucrose, and PEG3350 or poloxamer 188 at varying concentrations, at pH 5.9. The formulations were stored in glass vials and agitated by vortexing (speed setting=4) for 30 minutes, 60 minutes, or 120 minutes. The percentage of high molecular weight (HMW) species was then determined by size exclusion ultra high performance liquid chromatography (SE-UPLC).

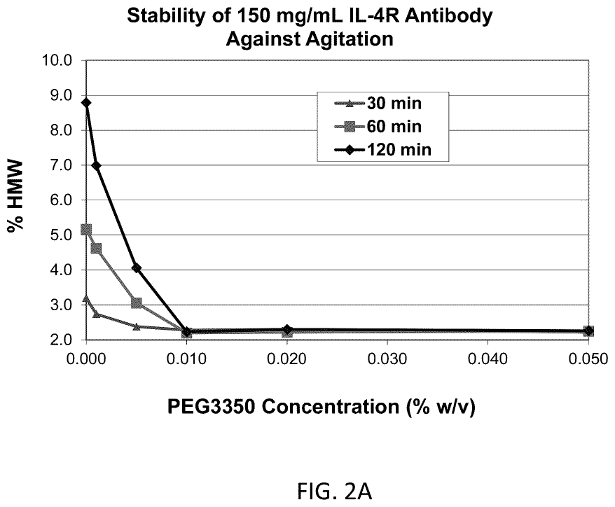

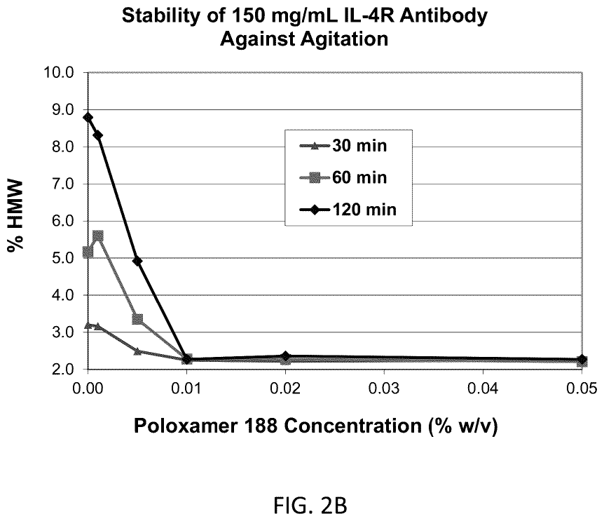

As shown in FIG. 2A, formulation of the antibody with at least 0.01% (w/v) PEG3350 prevented an observable increase of HMW species (quantitated by SE-UHPLC) due to the agitation. Lower amounts of PEG3350 (0.001% or 0.005%) were insufficient to prevent formation of HMW. Similarly, as shown in FIG. 2B, formulation of the antibody with at least 0.01% (w/v) poloxamer 188 prevented an observable increase of HMW species but HMW species were observed with lower amounts of poloxamer 188 (0.001% or 0.005%).

### Example 3: Thermal Stress Stability of Formulations Containing PEG3350 or Poloxamer 188

The thermal stress stability of IL-4R antibody formulations containing PEG3350 or poloxamer 188 was tested and compared to various IL-4R antibody formulations containing polysorbate. An anti-IL-4R antibody comprising the HCVR/LCVR amino acid sequence pair of SEQ ID NOs:1/2 at a concentration of 150 mg/mL was formulated with 20 mM histidine, 12.5 mM sodium acetate, 25 mM arginine-HCl, 5% w/v sucrose, and polysorbate 20, polysorbate 80, PEG3350 or poloxamer 188 at varying concentrations, at pH 5.9. These formulations were subjected to thermal stress (45° C.) for a period of up to 56 days, and the percentage of high molecular weight (HMW) species was determined by size exclusion ultra high performance liquid chromatography (SE-UPLC) at 7 days, at 14 days, at 28 days, at 42 days, and at 56 days.

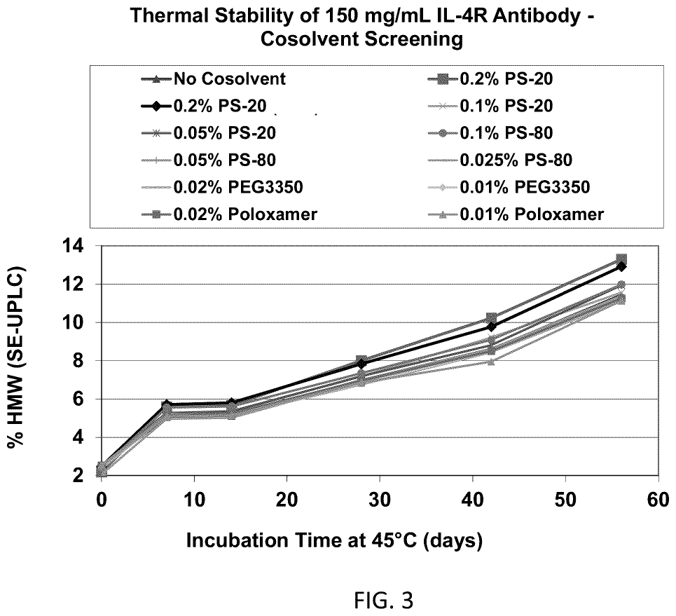

As shown in FIG. 3, antibody formulations comprising PEG3350 or poloxamer 188 at a concentration of 0.01% or 0.02% showed similar thermal stability to antibody formulations containing lower amounts of polysorbate (up to 0.1% w/v). By 28 days, the PEG3350- or poloxamer 188-containing formulations exhibited a lower percentage of HMW species than control formulations comprising 0.2% polysorbate 20.

### Example 4: Thermal Stress Stability of Formulations Containing Varying Concentrations of Poloxamer 188 or PEG3350

The thermal stress stability of IL-4R antibody formulations containing different concentrations of poloxamer 188 or PEG3350 was tested under thermal stress conditions (45° C.) for up to 3 months. An anti-IL-4R antibody comprising the HCVR/LCVR amino acid sequence pair of SEQ ID NOs:1/2 at a concentration of 150 mg/mL was formulated with 20 mM histidine, 12.5 mM sodium acetate, 25 mM arginine-HCl, 5% w/v sucrose, and surfactant [polysorbate 80 at 0.2% w/v, poloxamer 188 at 0.01%, 0.1%, or 0.5% w/v, or PEG3350 at 0.01%, 0.1%, or 0.5% w/v] at pH 5.9. These formulations were stored in glass vials at 45° C. for 1, 2, or 3 months. The percentage of drug product (antibody) recovered was determined by reverse phase ultra high performance liquid chromatography (RP-UPLC). The percentage of high molecular weight (HMW) species was determined by size exclusion ultra high performance liquid chromatography (SE-UPL). The formation of charge variant species was measured by cation exchange ultra performance liquid chromatography (CEX-UPLC). Micro-flow imaging (MFI) was used to measure subvisible particles.

Table 2 shows stability data for a formulation containing 0.2% (w/v) polysorbate 80 at 0,1, and 3 months. Tables 3, 4, and 5 show stability data for formulations containing 0.01%, 0.1%, and 0.5% poloxamer 188, respectively. Tables 6, 7, and 8 show stability data for formulations containing 0.01%, 0.1%, and 0.5% PEG3350, respectively. As shown in Tables 3 and 6, even the lowest tested concentrations of poloxamer 188 and PEG3350 (0.01%) provided a similar degree of protection as 0.2% polysorbate 80. The 0.01% poloxamer 188 formulation exhibited similar stability characteristics as 0.1% and 0.5% poloxamer 188 (see, Tables 3-5). The 0.01% PEG3350 formulation exhibited similar stability characteristics as 0.1% and 0.5% PEG3350 (see, Tables 6-8).

TABLE 2
Stability Data for Formulation with 0.2% (w/v) Polysorbate 80

| | | | Length of Storage at 45° C. (months) | | |
|---|---|---|---|---|---|
| Assay | | | 0 | 1 | 3 |
| % of Antibody Recovered | | | 100 | 98 | 94 |
| by RP-UPLC | | | | | |
| Purity by SE-UPLC | % HMW | | 2.1 | 10.3 | 26.6 |
| | % Main | | 97.2 | 88.7 | 70.7 |
| | % LMW | | 0.7 | 1.0 | 2.7 |
| Charge Variant | % Acidic | | 27.7 | 53.7 | 86.9 |
| Analysis by | % Main | | 62.5 | 41.0 | 9.5 |
| CEX-UPLC | % Basic | | 9.9 | 5.3 | 3.6 |
| MFI (# particles/mL) | 2-10 | μm | 125 | 1428 | 377 |
| | ≥10 | μm | 23 | 29 | 19 |
| | ≥25 | μm | 4 | 2 | 4 |

TABLE 3
Stability Data for Formulation with 0.01% (w/v) Poloxamer 188

| | | | Length of Storage at 45° C. (months) | | |
|---|---|---|---|---|---|
| Assay | | | 0 | 1 | 3 |
| % of Antibody Recovered | | | 100 | 100 | 98 |
| by RP-UPLC | | | | | |
| Purity by SE-UPLC | % HMW | | 2.1 | 9.1 | 23.7 |
| | % Main | | 97.2 | 90.0 | 73.5 |
| | % LMW | | 0.7 | 0.9 | 2.7 |
| Charge Variant | % Acidic | | 28.4 | 51.7 | 86.6 |
| Analysis by | % Main | | 62.1 | 43.1 | 8.9 |
| CEX-UPLC | % Basic | | 9.5 | 5.3 | 4.5 |
| MFI (# particles/mL) | 2-10 | μm | 79 | 142 | 396 |
| | ≥10 | μm | 17 | 17 | 8 |
| | ≥25 | μm | 0 | 6 | 0 |

TABLE 4
Stability Data for Formulation with 0.1% (w/v) Poloxamer 188

| | | | Length of Storage at 45° C. (months) | | |
|---|---|---|---|---|---|
| Assay | | | 0 | 1 | 3 |
| % of Antibody Recovered | | | 100 | 99 | 100 |
| by RP-UPLC | | | | | |
| Purity by SE-UPLC | % HMW | | 2.1 | 9.1 | 23.7 |
| | % Main | | 97.3 | 90.0 | 73.6 |
| | % LMW | | 0.6 | 0.9 | 2.8 |
| Charge Variant | % Acidic | | 28.1 | 51.9 | 84.0 |
| Analysis by | % Main | | 62.1 | 46.2 | 8.7 |
| CEX-UPLC | % Basic | | 9.8 | 1.9 | 7.4 |
| MFI (# particles/mL) | 2-10 | μm | 69 | 540 | 811 |
| | ≥10 | μm | 0 | 17 | 25 |
| | ≥25 | μm | 0 | 0 | 0 |

TABLE 5
Stability Data for Formulation with 0.5% (w/v) Poloxamer 188

| | | | Length of Storage at 45° C. (months) | | |
|---|---|---|---|---|---|
| Assay | | | 0 | 1 | 3 |
| % of Antibody Recovered | | | 100 | 100 | 100 |
| by RP-UPLC | | | | | |
| Purity by SE-UPLC | % HMW | | 2.1 | 9.1 | 24.2 |
| | % Main | | 97.3 | 90.0 | 73.1 |
| | % LMW | | 0.6 | 0.9 | 2.7 |
| Charge Variant | % Acidic | | 29.5 | 50.8 | 86.6 |
| Analysis by | % Main | | 61.5 | 44.5 | 9.0 |
| CEX-UPLC | % Basic | | 9.1 | 4.8 | 4.4 |
| MFI (# particles/mL) | 2-10 | μm | 179 | 805 | 1887 |
| | ≥10 | μm | 10 | 8 | 307 |
| | ≥25 | μm | 0 | 2 | 61 |

TABLE 6
Stability Data for Formulation with 0.01% (w/v) PEG3350

| | | | Length of Storage at 45° C. (months) | | |
|---|---|---|---|---|---|
| Assay | | | 0 | 1 | 3 |
| % of Antibody Recovered | | | 100 | 102 | 100 |
| by RP-UPLC | | | | | |
| Purity by SE-UPLC | % HMW | | 2.1 | 9.3 | 23.6 |
| | % Main | | 97.4 | 89.9 | 73.6 |
| | % LMW | | 0.6 | 0.9 | 2.7 |
| Charge Variant | % Acidic | | 29.4 | 51.6 | 86.4 |
| Analysis by | % Main | | 62.2 | 46.1 | 11.9 |
| CEX-UPLC | % Basic | | 8.5 | 2.4 | 1.8 |
| MFI (# particles/mL) | 2-10 | μm | 42 | 494 | 2085 |
| | ≥10 | μm | 6 | 15 | 206 |
| | ≥25 | μm | 0 | 6 | 40 |

TABLE 7
Stability Data for Formulation with 0.1% (w/v) PEG3350

| | | | Length of Storage at 45° C. (months) | | |
|---|---|---|---|---|---|
| Assay | | | 0 | 1 | 3 |
| % of Antibody Recovered | | | 100 | 97 | 92 |
| by RP-UPLC | | | | | |
| Purity by SE-UPLC | % HMW | | 2.1 | 9.3 | 22.4 |
| | % Main | | 97.4 | 89.8 | 74.9 |
| | % LMW | | 0.6 | 0.9 | 2.8 |
| Charge Variant | % Acidic | | 32.1 | 51.0 | 85.7 |
| Analysis by | % Main | | 60.6 | 45.6 | 10.9 |
| CEX-UPLC | % Basic | | 7.4 | 3.4 | 3.5 |
| MFI (# particles/mL) | 2-10 | μm | 94 | 313 | 2058 |
| | ≥10 | μm | 2 | 15 | 159 |
| | ≥25 | μm | 0 | 4 | 21 |

TABLE 8
Stability Data for Formulation with 0.5% (w/v) PEG3350

| | | | Length of Storage at 45° C. (months) | | |
|---|---|---|---|---|---|
| Assay | | | 0 | 1 | 3 |
| % of Antibody Recovered | | | 100 | 100 | 98 |
| by RP-UPLC | | | | | |
| Purity by SE-UPLC | % HMW | | 2.1 | 9.2 | 23.9 |
| | % Main | | 97.3 | 89.8 | 73.4 |
| | % LMW | | 0.6 | 0.9 | 2.7 |
| Charge Variant | % Acidic | | 29.7 | 52.0 | 83.6 |
| Analysis by | % Main | | 62.0 | 45.7 | 10.5 |
| CEX-UPLC | % Basic | | 8.4 | 2.3 | 6.0 |
| MFI (# particles/mL) | 2-10 | μm | 121 | 1289 | 2654 |
| | ≥10 | μm | 0 | 44 | 279 |
| | ≥25 | μm | 0 | 25 | 31 |

### Example 5: Stability Data for Storage and Stress Conditions for Formulations Containing Varying Concentrations of Poloxamer 188 or PEG3350

The stability of IL-4R antibody formulations in having varying concentrations of poloxamer 188 or PEG3350 was tested under storage conditions of 5° C. for up to 18 months, accelerated conditions (25° C. for up to 6 months), thermal stress conditions (45° C. for up to 2 months), and agitation stress conditions. An anti-IL-4R antibody comprising the HCVR/LCVR amino acid sequence pair of SEQ ID NOs:1/2 at a concentration of 150 mg/mL was formulated with 20 mM histidine, 12.5 mM sodium acetate, 25 mM arginine-HCl, and 5% w/v sucrose, either in the absence of surfactant or in the presence of poloxamer 188 or PEG3350 at 0.02%, 0.04%, or 0.1% w/v, at pH 5.9. These formulations were stored in Nuova Ompi 2.25 mL syringes. The percentage of HMW species was determined by SE-UPLC. The formation of charge variant species was measured by CEX-UPLC. Membrane microscopy and/or MFI was used to measure the number of particles in the container.

Tables 9-11 show data for a formulation without surfactant. Tables 12-14, 15-17, and 18-20 show stability data for formulations containing 0.02%, 0.04%, and 0.1% (w/v) poloxamer 188, respectively. Tables 21-23, 24-26, and 27-29 show stability data for formulations containing 0.02%, 0.04%, and 0.1% (w/v) PEG3350, respectively. As shown in Tables 12, 15, and 18, formulations containing 0.02%, 0.04%, or 0.1% (w/v) poloxamer had significantly fewer particles after prolonged storage at 5° C. (up to 18 months). Similarly, as shown in Tables 21, 24, and 27, formulations containing 0.02%, 0.04%, or 0.1% PEG3350 had significantly fewer particles after prolonged storage at 5° C. (up to 18 months). Under accelerated conditions of 25° C. or thermal stress conditions of 45° C., formulations containing 0.02%, 0.04%, or 0.1% (w/v) poloxamer 188 or PEG3350 had significantly fewer subvisible particles than a formulation without surfactant (see, Tables 10, 13, 16, 19, 22, 25, and 28).

The formulations were subjected to agitation stress for 24 hours at 25° C., then stored as follows: at 5° C. for 6, 12, or 18 months, at 25° C. for 6 months, or at 45° C. for 1 or 2 months. Formulations containing 0.02%, 0.04%, or 0.1% (w/v) poloxamer 188 or PEG3350 had a significantly less formation of HMW species after 24 hours of agitation at 25° C., as compared to a formulation lacking surfactant. Additionally, the formulations containing 0.02%, 0.04%, or 0.1% (w/v) poloxamer 188 or PEG3350 exhibited lower percentages of HMW species for 24 hours of agitation followed by prolonged storage at 5° C. or 25° C. conditions, as compared to a formulation lacking surfactant, indicative of improved stability. See, Tables 11, 14, 17, 20, 23, 26, and 29.

TABLE 9
Storage Stability Data (5° C.) for Formulation Without Surfactant

| | | | | Length of Storage at 5° C. (months) | | | | | |
|---|---|---|---|---|---|---|---|---|---|
| Assay | | | t = 0 | 1 | 3 | 6 | 9 | 12 | 18 |
| Purity by SE-UPLC | % HMW | | 2.1 | 2.2 | 2.3 | 2.6 | 2.6 | 2.7 | 2.9 |
| | % Main | | 96.8 | 97.6 | 97.7 | 96.5 | 96.5 | 96.6 | 96.3 |
| | % LMW | | 1.1 | 0.3 | 0.0 | 1.0 | 0.9 | 0.7 | 0.9 |
| Charge Variant Analysis by | % Acidic | | 27.1 | 26.2 | 25.8 | 26.4 | N/A | 26.3 | 25.7 |
| CEX-UPLC | % Main | | 58.7 | 61.1 | 61.8 | 60.4 | N/A | 60.8 | 61.9 |
| | % Basic | | 14.2 | 12.7 | 12.4 | 13.3 | N/A | 13.0 | 12.4 |
| Membrane Microscopy | ≥10 | μm | N/A | N/A | N/A | 72 | N/A | 51 | 120 |
| (# particles/container) ^{a} | ≥25 | μm | N/A | N/A | N/A | 11 | N/A | 30 | 27 |
| MFI (# particles/ | 2-300 | μm | 13600 | N/A | N/A | 17782 | N/A | 15175 | 38790 |
| container) | 5-300 | μm | 4895 | N/A | N/A | 6367 | N/A | 4859 | 17738 |
| (aspect ratio ≤ 0.85 | 10-300 | μm | 2491 | N/A | N/A | 3024 | N/A | 2044 | 9521 |
| applied) | 25-300 | μm | 533 | N/A | N/A | 511 | N/A | 406 | 2368 |
| | 50-300 | μm | 47 | N/A | N/A | 59 | N/A | 51 | 265 |

^{a} Microscopy USP specifications: ≥10 μm sizes ≤ 3000 particles/container; ≥25 μm sizes ≤ 300 particles/container
HMW = high molecular weight;
LMW = low molecular weight;
N/A = not available

TABLE 10
Accelerated Storage and Thermal Stress Stability Data for Formulation Without Surfactant

| | | | | Length of Incubation at | | | Length of Incubation at | | |
|---|---|---|---|---|---|---|---|---|---|
| | | | | 25° C. (months) | | | 45° C.(months) | | |
| Assay | | | t = 0 | 1 | 3 | 6 | 0.5 | 1 | 2 |
| Purity by SE-UPLC | % HMW | | 2.1 | 2.7 | 3.2 | 3.8 | 3.6 | 4.7 | 8.3 |
| | % Main | | 96.8 | 96.9 | 96.38 | 94.9 | 95.4 | 93.8 | 89.3 |
| | % LMW | | 1.1 | 0.4 | 0.0 | 1.3 | 1.0 | 1.5 | 2.4 |
| Charge Variant Analysis | % Acidic | | 27.1 | 26.5 | 28.9 | 32.3 | 31.2 | 37.5 | 62.5 |
| by CEX-UPLC | % Main | | 58.7 | 59.5 | 57.9 | 53.8 | 56.3 | 50.3 | 27.0 |
| | % Basic | | 14.2 | 14.1 | 13.2 | 13.9 | 12.5 | 12.2 | 10.5 |
| Membrane Microscopy | ≥10 | μm | N/A | N/A | N/A | 49 | N/A | N/A | N/A |
| (# particles/container) ^{a} | ≥25 | μm | N/A | N/A | N/A | 11 | N/A | N/A | N/A |
| MFI (# particles/ | 2-300 | μm | 13600 | N/A | N/A | 19813 | N/A | 9066 | N/A |
| container) | 5-300 | μm | 4895 | N/A | N/A | 8827 | N/A | 2510 | N/A |
| (aspect ratio ≤ 0.85 | 10-300 | μm | 2491 | N/A | N/A | 4347 | N/A | 1117 | N/A |
| applied) | 25-300 | μm | 533 | N/A | N/A | 639 | N/A | 163 | N/A |
| | 50-300 | μm | 47 | N/A | N/A | 39 | N/A | 7 | N/A |

^{a} Microscopy USP specifications: ≥10 μm sizes ≤ 3000 particles/container; ≥25 μm sizes ≤ 300 particles/container
HMW = high molecular weight;
LMW = low molecular weight;
N/A = not available

TABLE 11
Agitation Stress Stability Data for Formulation Without Surfactant

| | | | | | | | 24 hours | | |
|---|---|---|---|---|---|---|---|---|---|
| | | | | | | | agitation*, | | |
| | | | | | | | then | 24 hours | |
| | | | 24 hours agitation*, then | | | | storage at | agitation*, then | |
| | | | storage at | | | | 25° C. | storage at | |
| | | | 5° C. (months) | | | | (months) | 45° C. (months) | |
| Assay | | t = 0 | T0 | 6 | 12 | 18 | 6 | 1 | 2 |
| Purity by SE-UPLC | % HMW | 2.1 | 9.4 | 8.5 | 9.1 | 7.2 | 7.9 | 5.3 | 8.3 |
| | % Main | 96.8 | 90.6 | 90.6 | 90.3 | 92.0 | 91.1 | 93.4 | 89.5 |
| | % LMW | 1.1 | 0.0 | 0.9 | 0.6 | 0.8 | 1.0 | 1.3 | 2.3 |
| Charge Variant Analysis | % Acidic | 27.1 | 26.7 | 25.8 | 26.4 | 26.2 | 32.4 | 37.5 | 61.5 |
| by CEX-UPLC | % Main | 58.7 | 59.8 | 61.0 | 60.7 | 61.8 | 53.9 | 49.9 | 26.8 |
| | % Basic | 14.2 | 13.5 | 13.3 | 13.0 | 12.1 | 13.7 | 12.7 | 11.7 |

*Agitation for 24 hours at 25° C. followed by storage at indicated temperature
HMW = high molecular weight;
LMW = low molecular weight

TABLE 12
Storage Stability Data (5° C.) for Formulation with 0.02% (w/v) Poloxamer 188

| | | | | Length of Storage at 5° C. (months) | | | | | |
|---|---|---|---|---|---|---|---|---|---|
| Assay | | | t = 0 | 1 | 3 | 6 | 9 | 12 | 18 |
| Purity by SE-UPLC | % HMW | | 2.0 | 2.2 | 2.3 | 2.5 | 2.6 | 2.7 | 2.8 |
| | % Main | | 97.1 | 97.6 | 97.7 | 96.5 | 96.5 | 96.6 | 96.4 |
| | % LMW | | 0.8 | 0.2 | 0.0 | 1.0 | 0.9 | 0.7 | 0.9 |
| Charge Variant Analysis by | % Acidic | | 26.9 | 27.5 | 26.4 | 26.5 | N/A | 26.1 | 25.9 |
| CEX-UPLC | % Main | | 58.4 | 59.2 | 60.5 | 59.9 | N/A | 60.3 | 61.8 |
| | % Basic | | 14.8 | 13.3 | 13.1 | 13.6 | N/A | 13.6 | 12.3 |
| Membrane Microscopy | ≥10 | μm | N/A | N/A | N/A | 36 | N/A | 42 | 86 |
| (# particles/container) ^{a} | ≥25 | μm | N/A | N/A | N/A | 17 | N/A | 17 | 21 |
| MFI (# particles/ | 2-300 | μm | 27602 | N/A | N/A | 6120 | N/A | 14265 | 9781 |
| container) | 5-300 | μm | 8871 | N/A | N/A | 992 | N/A | 3355 | 1560 |
| (aspect ratio ≤ 0.85 | 10-300 | μm | 3440 | N/A | N/A | 277 | N/A | 1349 | 622 |
| applied) | 25-300 | μm | 294 | N/A | N/A | 10 | N/A | 201 | 43 |
| | 50-300 | μm | 11 | N/A | N/A | 2 | N/A | 38 | 2 |

^{a} Microscopy USP specifications: ≥10 μm sizes ≤ 3000 particles/container; ≥25 μm sizes ≤ 300 particles/container
HMW = high molecular weight;
LMW = low molecular weight;
N/A = not available;

TABLE 13
Accelerated Storage and Thermal Stress Stability Data
for Formulation with 0.02% (w/v) Poloxamer 188

| | | | | Length of Incubation at | | | Length of Incubation at | | |
|---|---|---|---|---|---|---|---|---|---|
| | | | | 25° C. (months) | | | 45° C.(months) | | |
| Assay | | | t = 0 | 1 | 3 | 6 | 0.5 | 1 | 2 |
| Visual Appearance | | | | | | | | | |
| Purity by SE-UPLC | % HMW | | 2.0 | 2.7 | 3.1 | 3.8 | 3.8 | 4.8 | 8.2 |
| | % Main | | 97.1 | 97.1 | 96.9 | 95.1 | 95.1 | 93.8 | 89.1 |
| | % LMW | | 0.8 | 0.3 | 0.0 | 1.2 | 1.1 | 1.4 | 2.6 |
| Charge Variant Analysis | % Acidic | | 26.9 | 26.8 | 29.2 | 32.1 | 31.5 | 37.9 | 60.0 |
| by CEX-UPLC | % Main | | 58.4 | 59.0 | 55.4 | 54.3 | 55.3 | 49.1 | 26.6 |
| | % Basic | | 14.8 | 14.2 | 15.4 | 13.6 | 13.3 | 13.0 | 13.4 |
| Membrane Microscopy | ≥10 | μm | N/A | N/A | N/A | 36 | N/A | N/A | N/A |
| (# particles/container) ^{a} | ≥25 | μm | N/A | N/A | N/A | 13 | N/A | N/A | N/A |
| MFI (# particles/ | 2-300 | μm | 27602 | N/A | N/A | 5786 | N/A | 4584 | N/A |
| container) | 5-300 | μm | 8871 | N/A | N/A | 457 | N/A | 455 | N/A |
| (aspect ratio ≤ 0.85 | 10-300 | μm | 3440 | N/A | N/A | 198 | N/A | 209 | N/A |
| applied) | 25-300 | μm | 294 | N/A | N/A | 10 | N/A | 14 | N/A |
| | 50-300 | μm | 11 | N/A | N/A | 2 | N/A | 0 | N/A |

^{a} Microscopy USP specifications: ≥10 μm sizes ≤ 3000 particles/container; ≥25 μm sizes ≤ 300 particles/container
HMW = high molecular weight;
LMW = low molecular weight;
N/A = not available

TABLE 14
Agitation Stress Stability Data for Formulation with 0.02% (w/v) Poloxamer 188

| | | | | | | | 24 hours | | |
|---|---|---|---|---|---|---|---|---|---|
| | | | | | | | agitation*, | | |
| | | | | | | | then | 24 hours | |
| | | | 24 hours agitation*, then | | | | storage at | agitation*, then | |
| | | | storage at | | | | 25° C. | storage at | |
| | | | 5° C. (months) | | | | (months) | 45° C. (months) | |
| Assay | | t = 0 | T0 | 6 | 12 | 18 | 6 | 1 | 2 |
| Purity by SE-UPLC | % HMW | 2.0 | 4.1 | 3.0 | 3.0 | 3.0 | 3.9 | 4.9 | 8.3 |
| | % Main | 97.1 | 95.7 | 96.1 | 96.3 | 95.9 | 95.0 | 93.8 | 89.3 |
| | % LMW | 0.8 | 0.1 | 0.9 | 0.7 | 0.9 | 1.2 | 1.4 | 2.5 |
| Charge Variant Analysis | % Acidic | 26.9 | 26.7 | 26.0 | 26.2 | 25.8 | 32.5 | 37.4 | 59.4 |
| by CEX-UPLC | % Main | 58.4 | 59.3 | 60.8 | 60.1 | 61.6 | 53.6 | 48.9 | 26.9 |
| | % Basic | 14.8 | 14.0 | 13.3 | 13.8 | 12.6 | 13.9 | 13.7 | 13.7 |

*Agitation for 24 hours at 25° C. followed by storage at indicated temperature
HMW = high molecular weight;
LMW = low molecular weight;
N/A = not available

TABLE 15
Storage Stability Data (5° C.) for Formulation with 0.04% (w/v) Poloxamer 188

| | | | | Length of Storage at 5° C. (months) | | | | | |
|---|---|---|---|---|---|---|---|---|---|
| Assay | | | t = 0 | 1 | 3 | 6 | 9 | 12 | 18 |
| Purity by SE-UPLC | % HMW | | 2.1 | 2.2 | 2.3 | 2.6 | 2.6 | 2.7 | 2.8 |
| | % Main | | 97.1 | 97.6 | 97.7 | 96.5 | 96.5 | 96.6 | 96.3 |
| | % LMW | | 0.8 | 0.2 | 0.0 | 1.0 | 0.9 | 0.7 | 0.8 |
| Charge Variant Analysis | % Acidic | | 26.9 | 27.7 | 26.4 | 26.1 | N/A | 25.9 | 25.6 |
| by CEX-UPLC | % Main | | 58.3 | 59.0 | 60.3 | 59.4 | N/A | 60.8 | 62.0 |
| | % Basic | | 14.8 | 13.4 | 13.3 | 14.5 | N/A | 13.3 | 12.4 |
| Membrane Microscopy | ≥10 | μm | N/A | N/A | N/A | 34 | N/A | 25 | 40 |
| (# particles/container) ^{a} | ≥25 | μm | N/A | N/A | N/A | 2 | N/A | 8 | 4 |
| MFI (# particles/container) | 2-300 | μm | 7067 | N/A | N/A | 2747 | N/A | 15777 | 19227 |
| (aspect ratio ≤0.85 applied) | 5-300 | μm | 772 | N/A | N/A | 171 | N/A | 1698 | 2034 |
| | 10-300 | μm | 289 | N/A | N/A | 54 | N/A | 734 | 720 |
| | 25-300 | μm | 8 | N/A | N/A | 5 | N/A | 33 | 23 |
| | 50-300 | μm | 0 | N/A | N/A | 0 | N/A | 3 | 0 |

^{a} Microscopy USP specifications: ≥10 μm sizes ≤3000 particles/container; ≥25 μm sizes ≤300 particles/container
HMW = high molecular weight;
LMW = low molecular weight;
N/A = not available

TABLE 16
Accelerated Storage and Thermal Stress Stability Data
for Formulation with 0.04% (w/v) Poloxamer 188

| | | | | Length of Incubation | | | Length of Incubation | | |
|---|---|---|---|---|---|---|---|---|---|
| | | | | at 25° C. (months) | | | at 45° C.(months) | | |
| Assay | | | t = 0 | 1 | 3 | 6 | 0.5 | 1 | 2 |
| Purity by SE-UPLC | % HMW | | 2.1 | 2.7 | 2.9 | 3.8 | 3.8 | 4.8 | 8.7 |
| | % Main | | 97.1 | 97.2 | 97.1 | 95.0 | 95.2 | 93.7 | 89.0 |
| | % LMW | | 0.8 | 0.1 | 0.0 | 1.1 | 1.0 | 1.5 | 2.3 |
| Charge Variant Analysis | % Acidic | | 26.9 | 26.4 | 29.5 | 32.3 | 31.3 | 37.8 | 60.3 |
| by CEX-UPLC | % Main | | 58.3 | 57.9 | 54.8 | 54.1 | 55.6 | 48.5 | 24.2 |
| | % Basic | | 14.8 | 15.6 | 15.8 | 13.6 | 13.0 | 13.8 | 15.5 |
| Membrane Microscopy | ≥10 | μm | N/A | N/A | N/A | 15 | N/A | N/A | N/A |
| (# particles/container) ^{a} | ≥25 | μm | N/A | N/A | N/A | 0 | N/A | N/A | N/A |
| MFI (# particles/container) | 2-300 | μm | 7067 | N/A | N/A | 7042 | N/A | 7510 | N/A |
| (aspect ratio ≤ 0.85 applied) | 5-300 | μm | 772 | N/A | N/A | 550 | N/A | 377 | N/A |
| | 10-300 | μm | 289 | N/A | N/A | 253 | N/A | 98 | N/A |
| | 25-300 | μm | 8 | N/A | N/A | 5 | N/A | 3 | N/A |
| | 50-300 | μm | 0 | N/A | N/A | 2 | N/A | 0 | N/A |

^{a} Microscopy USP specifications: ≥10 μm sizes ≤3000 particles/container; ≥25 μm sizes ≤300 particles/container
HMW = high molecular weight;
LMW = low molecular weight;
N/A = not available

TABLE 17
Agitation Stress Stability Data for Formulation with 0.04% (w/v) Poloxamer 188

| | | | | | | | 24 hours | 24 hours | |
|---|---|---|---|---|---|---|---|---|---|
| | | | | | | | agitation*, | agitation*, | |
| | | | | | | | then storage | then storage | |
| | | | 24 hours agitation*, then | | | | at 25° C. | at 45° C. | |
| | | | storage at 5° C. (months) | | | | (months) | (months) | |
| Assay | | t = 0 | T0 | 6 | 12 | 18 | 6 | 1 | 2 |
| Purity by SE-UPLC | % HMW | 2.1 | 2.0 | 2.6 | 2.8 | 2.9 | 3.9 | 5.0 | 8.8 |
| | % Main | 97.1 | 95.2 | 96.5 | 96.5 | 96.2 | 95.0 | 93.7 | 89.0 |
| | % LMW | 0.8 | 2.7 | 0.1 | 0.7 | 0.9 | 1.2 | 1.3 | 2.3 |
| Charge Variant Analysis | % Acidic | 26.9 | 26.9 | 26.3 | 26.4 | 26.3 | 32.3 | 38.0 | 60.2 |
| by CEX-UPLC | % Main | 58.3 | 58.3 | 60.0 | 59.9 | 61.2 | 54.3 | 48.3 | 25.4 |
| | % Basic | 14.8 | 14.8 | 13.7 | 13.7 | 12.6 | 13.4 | 13.8 | 14.4 |

*Agitation for 24 hours at 25° C. followed by storage at indicated temperature
HMW = high molecular weight;
LMW = low molecular weight

TABLE 18
Storage Stability Data (5° C.) for Formulation with 0.1% (w/v) Poloxamer 188

| | | | | Length of Storage at 5° C. (months) | | | | | |
|---|---|---|---|---|---|---|---|---|---|
| Assay | | | t = 0 | 1 | 3 | 6 | 9 | 12 | 18 |
| Purity by SE-UPLC | % HMW | | 2.1 | 2.2 | 2.3 | 2.6 | 2.6 | 2.7 | 2.9 |
| | % Main | | 97.0 | 97.6 | 97.8 | 96.5 | 96.5 | 96.6 | 96.3 |
| | % LMW | | 1.0 | 0.2 | 0.0 | 0.9 | 0.9 | 0.7 | 0.9 |
| Charge Variant Analysis | % Acidic | | 27.0 | 27.6 | 26.2 | 26.0 | N/A | 26.3 | 25.3 |
| by CEX-UPLC | % Main | | 58.2 | 59.1 | 59.9 | 59.8 | N/A | 60.7 | 62.3 |
| | % Basic | | 14.8 | 13.3 | 14.0 | 14.2 | N/A | 13.0 | 12.4 |
| Membrane Microscopy | ≥10 | μm | N/A | N/A | N/A | 84 | N/A | 34 | 114 |
| (# particles/container) ^{a} | ≥25 | μm | N/A | N/A | N/A | 19 | N/A | 4 | 30 |
| MFI (# particles/container) | 2-300 | μm | 3424 | N/A | N/A | 3325 | N/A | 6985 | 18856 |
| (aspect ratio ≤0.85 applied) | 5-300 | μm | 215 | N/A | N/A | 217 | N/A | 426 | 1802 |
| | 10-300 | μm | 119 | N/A | N/A | 131 | N/A | 204 | 894 |
| | 25-300 | μm | 5 | N/A | N/A | 3 | N/A | 8 | 16 |
| | 50-300 | μm | 0 | N/A | N/A | 2 | N/A | 3 | 3 |

^{a} Microscopy USP specifications: ≥10 μm sizes ≤3000 particles/container; ≥25 μm sizes ≤300 particles/container
HMW = high molecular weight;
LMW = low molecular weight;
N/A = not available

TABLE 19
Accelerated Storage and Thermal Stress Stability Data
for Formulation with 0.1% (w/v) Poloxamer 188

| | | | | Length of Incubation | | | Length of Incubation | | |
|---|---|---|---|---|---|---|---|---|---|
| | | | | at 25° C. (months) | | | at 45° C.(months) | | |
| Assay | | | t = 0 | 1 | 3 | 6 | 0.5 | 1 | 2 |
| Purity by SE-UPLC | % HMW | | 2.1 | 2.7 | 3.0 | 3.8 | 3.8 | 4.7 | 8.3 |
| | % Main | | 97.0 | 97.2 | 97.0 | 95.0 | 95.2 | 94.0 | 89.4 |
| | % LMW | | 1.0 | 0.1 | 0.0 | 1.2 | 1.0 | 1.3 | 2.3 |
| Charge Variant Analysis | % Acidic | | 27.0 | 27.6 | 29.5 | 32.3 | 31.4 | 37.8 | 59.4 |
| by CEX-UPLC | % Main | | 58.2 | 57.8 | 54.3 | 53.8 | 54.9 | 49.0 | 25.9 |
| | % Basic | | 14.8 | 14.6 | 16.3 | 13.9 | 13.7 | 13.3 | 14.7 |
| Membrane Microscopy | ≥10 | μm | N/A | N/A | N/A | 38 | N/A | N/A | N/A |
| (# particles/container) ^{a} | ≥25 | μm | N/A | N/A | N/A | 8 | N/A | N/A | N/A |
| MFI (# particles/container) | 2-300 | μm | 3424 | N/A | N/A | 18633 | N/A | 7812 | N/A |
| (aspect ratio ≤0.85 applied) | 5-300 | μm | 215 | N/A | N/A | 1410 | N/A | 465 | N/A |
| | 10-300 | μm | 119 | N/A | N/A | 650 | N/A | 204 | N/A |
| | 25-300 | μm | 5 | N/A | N/A | 29 | N/A | 5 | N/A |
| | 50-300 | μm | 0 | N/A | N/A | 0 | N/A | 0 | N/A |

^{a} Microscopy USP specifications: ≥10 μm sizes ≤3000 particles/container; ≥25 μm sizes ≤300 particles/container
HMW = high molecular weight;
LMW = low molecular weight;
N/A = not available

TABLE 20
Agitation Stress Stability Data for Formulation with 0.1% (w/v) Poloxamer 188

| | | | | | | | 24 hours | 24 hours | |
|---|---|---|---|---|---|---|---|---|---|
| | | | | | | | agitation*, | agitation*, | |
| | | | | | | | then storage | then storage | |
| | | | 24 hours agitation*, then | | | | at 25° C. | at 45° C. | |
| | | | storage at 5° C. (months) | | | | (months) | (months) | |
| Assay | | t = 0 | T0 | 6 | 12 | 18 | 6 | 1 | 2 |
| Purity by SE-UPLC | % HMW | 2.1 | 2.1 | 2.6 | 2.8 | 2.9 | 3.9 | 4.9 | 8.4 |
| | % Main | 97.0 | 97.8 | 96.4 | 96.5 | 96.3 | 95.0 | 93.8 | 89.2 |
| | % LMW | 1.0 | 0.1 | 1.0 | 0.7 | 0.9 | 1.2 | 1.3 | 2.4 |
| Charge Variant Analysis | % Acidic | 27.0 | 26.8 | 26.6 | 26.4 | 26.4 | 32.3 | 38.1 | 58.9 |
| by CEX-UPLC | % Main | 58.2 | 58.0 | 60.0 | 60.3 | 60.6 | 54.0 | 47.5 | 26.6 |
| | % Basic | 14.8 | 15.2 | 13.4 | 13.4 | 13.0 | 13.7 | 14.4 | 14.5 |

*Agitation for 24 hours at 25° C. followed by storage at indicated temperature
HMW = high molecular weight;
LMW = low molecular weight

TABLE 21
Storage Stability Data (5° C.) for Formulation with 0.02% (w/v) PEG3350

| | | | | Length of Storage at 5° C. (months) | | | | | |
|---|---|---|---|---|---|---|---|---|---|
| Assay | | | t = 0 | 1 | 3 | 6 | 9 | 12 | 18 |
| Purity by SE-UPLC | % HMW | | 2.0 | 2.2 | 2.3 | 2.5 | 2.7 | 2.7 | 2.8 |
| | % Main | | 97.1 | 97.8 | 97.7 | 96.5 | 96.5 | 96.6 | 96.3 |
| | % LMW | | 0.9 | 0.0 | 0.0 | 0.9 | 0.9 | 0.7 | 0.9 |
| Charge Variant Analysis | % Acidic | | 26.9 | 25.7 | 25.9 | 25.9 | N/A | 26.1 | 26.0 |
| by CEX-UPLC | % Main | | 58.5 | 61.3 | 60.4 | 60.7 | N/A | 60.5 | 61.8 |
| | % Basic | | 14.6 | 13.0 | 13.7 | 13.5 | N/A | 13.3 | 12.2 |
| Membrane Microscopy | ≥10 | μm | N/A | N/A | N/A | 15 | N/A | 23 | 25 |
| (# particles/container) ^{a} | ≥25 | μm | N/A | N/A | N/A | 0 | N/A | 6 | 8 |
| MFI (# particles/container) | 2-300 | μm | 15027 | N/A | N/A | 10791 | N/A | 23589 | 22239 |
| (aspect ratio ≤0.85 applied) | 5-300 | μm | 6840 | N/A | N/A | 2297 | N/A | 8189 | 6644 |
| | 10-300 | μm | 3540 | N/A | N/A | 1021 | N/A | 3571 | 2581 |
| | 25-300 | μm | 612 | N/A | N/A | 144 | N/A | 444 | 386 |
| | 50-300 | μm | 18 | N/A | N/A | 8 | N/A | 18 | 51 |

^{a} Microscopy USP specifications: ≥10 μm sizes ≤3000 particles/container; ≥25 μm sizes ≤300 particles/container
HMW = high molecular weight;
LMW = low molecular weight;
N/A = not available

TABLE 22
Accelerated Storage and Thermal Stress Stability
Data for Formulation with 0.02% (w/v) PEG3350

| | | | | Length of Incubation | | | Length of Incubation | | |
|---|---|---|---|---|---|---|---|---|---|
| | | | | at 25° C. (months) | | | at 45° C.(months) | | |
| Assay | | | t = 0 | 1 | 3 | 6 | 0.5 | 1 | 2 |
| Purity by SE-UPLC | % HMW | | 2.0 | 2.6 | 3.1 | 3.8 | 3.7 | 4.9 | 8.6 |
| | % Main | | 97.1 | 97.1 | 96.9 | 95.0 | 95.4 | 93.8 | 89.2 |
| | % LMW | | 0.9 | 0.3 | 0.0 | 1.3 | 0.9 | 1.4 | 2.3 |
| Charge Variant Analysis | % Acidic | | 26.9 | 27.0 | 29.4 | 32.3 | 31.2 | 37.5 | 60.9 |
| by CEX-UPLC | % Main | | 58.5 | 58.6 | 55.3 | 53.8 | 56.3 | 50.0 | 25.7 |
| | % Basic | | 14.6 | 14.4 | 15.3 | 13.9 | 12.5 | 12.5 | 13.4 |
| Membrane Microscopy | ≥10 | μm | N/A | N/A | N/A | 49 | N/A | N/A | N/A |
| (# particles/container) ^{a} | ≥25 | μm | N/A | N/A | N/A | 15 | N/A | N/A | N/A |
| MFI (# particles/container) | 2-300 | μm | 15027 | N/A | N/A | 6011 | N/A | 9876 | N/A |
| (aspect ratio ≤0.85 applied) | 5-300 | μm | 6840 | N/A | N/A | 2448 | N/A | 1077 | N/A |
| | 10-300 | μm | 3540 | N/A | N/A | 10334 | N/A | 432 | N/A |
| | 25-300 | μm | 612 | N/A | N/A | 70 | N/A | 47 | N/A |
| | 50-300 | μm | 18 | N/A | N/A | 3 | N/A | 2 | N/A |

^{a} Microscopy USP specifications: ≥10 μm sizes ≤3000 particles/container; ≥25 μm sizes ≤300 particles/container
HMW = high molecular weight;
LMW = low molecular weight;
N/A = not available

TABLE 23
Agitation Stress Stability Data for Formulation with 0.02% (w/v) PEG3350

| | | | | | | | 24 hours | 24 hours | |
|---|---|---|---|---|---|---|---|---|---|
| | | | | | | | agitation*, | agitation*, | |
| | | | | | | | then storage | then storage | |
| | | | 24 hours agitation*, then | | | | at 25° C. | at 45° C. | |
| | | | storage at 5° C. (months) | | | | (months) | (months) | |
| Assay | | t = 0 | T0 | 6 | 12 | 18 | 6 | 1 | 2 |
| Visual Appearance | | | | | | | | | |
| Purity by SE-UPLC | % HMW | 2.0 | 2.9 | 2.9 | 2.8 | 2.9 | 3.9 | 4.9 | 8.5 |
| | % Main | 97.1 | 97.1 | 96.1 | 96.5 | 96.2 | 95.0 | 93.7 | 89.1 |
| | % LMW | 0.9 | 0.0 | 1.0 | 0.7 | 0.9 | 1.1 | 1.4 | 2.4 |
| Charge Variant Analysis | % Acidic | 26.9 | 26.7 | 26.0 | 261 | 26.1 | 32.7 | 37.6 | 60.3 |
| by CEX-UPLC | % Main | 58.5 | 59.3 | 60.7 | 60.4 | 61.6 | 54.0 | 49.6 | 25.8 |
| | % Basic | 14.6 | 14.0 | 13.3 | 13.6 | 12.3 | 13.4 | 12.8 | 13.9 |

*Agitation for 24 hours at 25° C. followed by storage at indicated temperature
HMW = high molecular weight;
LMW = low molecular weight

TABLE 24
Storage Stability Data (5° C.) for Formulation with 0.04% (w/v) PEG3350

| | | | | Length of Storage at 5° C. (months) | | | | | |
|---|---|---|---|---|---|---|---|---|---|
| Assay | | | t = 0 | 1 | 3 | 6 | 9 | 12 | 18 |
| Purity by SE-UPLC | % HMW | | 2.1 | 2.2 | 2.4 | 2.6 | 2.6 | 2.7 | 2.9 |
| | % Main | | 97.0 | 97.7 | 97.6 | 96.4 | 96.5 | 96.6 | 96.3 |
| | % LMW | | 1.0 | 0.1 | 0.0 | 1.0 | 0.9 | 0.7 | 0.9 |
| Charge Variant Analysis | % Acidic | | 26.8 | 26.2 | 25.8 | 26.4 | N/A | 26.1 | 25.2 |
| by CEX-UPLC | % Main | | 58.2 | 60.2 | 61.1 | 60.4 | N/A | 60.7 | 62.7 |
| | % Basic | | 14.9 | 13.7 | 13.1 | 13.2 | N/A | 13.2 | 12.1 |
| Membrane Microscopy | ≥10 | μm | N/A | N/A | N/A | 38 | N/A | 61 | 42 |
| (# particles/container) ^{a} | ≥25 | μm | N/A | N/A | N/A | 11 | N/A | 19 | 6 |
| MFI (# particles/container) | 2-300 | μm | 43797 | N/A | N/A | 18903 | N/A | 16022 | 22243 |
| (aspect ratio ≤ 0.85 applied) | 5-300 | μm | 6840 | N/A | N/A | 5833 | N/A | 4553 | 6873 |
| | 10-300 | μm | 10262 | N/A | N/A | 1951 | N/A | 1881 | 3082 |
| | 25-300 | μm | 940 | N/A | N/A | 67 | N/A | 204 | 367 |
| | 50-300 | μm | 16 | N/A | N/A | 5 | N/A | 2 | 15 |

^{a} Microscopy USP specifications: ≥10 μm sizes ≤3000 particles/container; ≥25 μm sizes ≤300 particles/container
HMW = high molecular weight;
LMW = low molecular weight;
N/A = not available

TABLE 25
Accelerated Storage and Thermal Stress Stability
Data for Formulation with 0.04% (w/v) PEG3350

| | | | | Length of Incubation | | | Length of Incubation | | |
|---|---|---|---|---|---|---|---|---|---|
| | | | | at 25° C. (months) | | | at 45° C.(months) | | |
| Assay | | | t = 0 | 1 | 3 | 6 | 0.5 | 1 | 2 |
| Purity by SE-UPLC | % HMW | | 2.1 | 2.6 | 3.0 | 3.8 | 3.8 | 4.6 | 8.2 |
| | % Main | | 97.0 | 97.3 | 97.0 | 95.1 | 95.2 | 94.0 | 89.4 |
| | % LMW | | 1.0 | 0.1 | 0.0 | 1.1 | 1.0 | 1.4 | 2.4 |
| Charge Variant Analysis | % Acidic | | 26.8 | 27.0 | 28.8 | 32.3 | 31.3 | 35.3 | 60.3 |
| by CEX-UPLC | % Main | | 58.2 | 58.3 | 56.0 | 53.6 | 55.0 | 51.1 | 24.3 |
| | % Basic | | 14.9 | 14.8 | 15.2 | 14.1 | 13.7 | 13.6 | 15.4 |
| Membrane Microscopy | ≥10 | μm | N/A | N/A | N/A | 80 | N/A | N/A | N/A |
| (# particles/container) ^{a} | ≥25 | μm | N/A | N/A | N/A | 25 | N/A | N/A | N/A |
| MFI (# particles/container) | 2-300 | μm | 43797 | N/A | N/A | 13611 | N/A | 5744 | N/A |
| (aspect ratio ≤0.85 applied) | 5-300 | μm | 6840 | N/A | N/A | 3628 | N/A | 475 | N/A |
| | 10-300 | μm | 10262 | N/A | N/A | 1581 | N/A | 188 | N/A |
| | 25-300 | μm | 940 | N/A | N/A | 103 | N/A | 18 | N/A |
| | 50-300 | μm | 16 | N/A | N/A | 2 | N/A | 2 | N/A |

^{a} Microscopy USP specifications: ≥10 μm sizes ≤3000 particles/container; ≥25 μm sizes ≤300 particles/container
HMW = high molecular weight;
LMW = low molecular weight;
N/A = not available

TABLE 26
Agitation Stress Stability Data for Formulation with 0.04% (w/v) PEG3350

| | | | | | | | 24 hours | 24 hours | |
|---|---|---|---|---|---|---|---|---|---|
| | | | | | | | agitation*, | agitation*, | |
| | | | | | | | then storage | then storage | |
| | | | 24 hours agitation*, then | | | | at 25° C. | at 45° C. | |
| | | | storage at 5° C. (months) | | | | (months) | (months) | |
| Assay | | t = 0 | T0 | 6 | 12 | 18 | 6 | 1 | 2 |
| Purity by SE-UPLC | % HMW | 2.1 | 2.1 | 2.6 | 2.7 | 2.9 | 3.9 | 4.8 | 8.3 |
| | % Main | 97.0 | 97.9 | 96.4 | 96.6 | 96.3 | 95.0 | 93.7 | 89.5 |
| | % LMW | 1.0 | 0.0 | 1.0 | 0.7 | 0.9 | 1.1 | 1.5 | 2.2 |
| Charge Variant Analysis | % Acidic | 26.8 | 26.8 | 26.6 | 26.3 | 25.9 | 32.6 | 35.8 | 59.5 |
| by CEX-UPLC | % Main | 58.2 | 57.5 | 60.0 | 60.3 | 61.7 | 53.7 | 50.0 | 26.4 |
| | % Basic | 14.9 | 15.8 | 13.5 | 13.4 | 12.4 | 13.6 | 14.2 | 14.2 |

*Agitation for 24 hours at 25° C. followed by storage at indicated temperature
HMW = high molecular weight;
LMW = low molecular weight

TABLE 27
Storage Stability Data (5° C.) for Formulation with 0.1% (w/v) PEG3350

| | | | | Length of Storage at 5° C. (months) | | | | | |
|---|---|---|---|---|---|---|---|---|---|
| Assay | | | t = 0 | 1 | 3 | 6 | 9 | 12 | 18 |
| Purity by SE-UPLC | % HMW | | 2.0 | 2.2 | 2.3 | 2.6 | 2.6 | 2.7 | 2.9 |
| | % Main | | 97.0 | 97.7 | 97.7 | 96.5 | 96.5 | 96.6 | 96.3 |
| | % LMW | | 0.9 | 0.2 | 0.0 | 0.9 | 0.9 | 0.7 | 0.8 |
| Charge Variant Analysis | % Acidic | | 26.9 | 27.7 | 26.5 | 26.3 | N/A | 26.3 | 26.1 |
| by CEX-UPLC | % Main | | 58.2 | 59.1 | 60.0 | 59.5 | N/A | 60.4 | 61.8 |
| | % Basic | | 15.0 | 13.3 | 13.5 | 14.2 | N/A | 13.3 | 12.1 |
| Membrane Microscopy | ≥10 | μm | N/A | N/A | N/A | 99 | N/A | 36 | 38 |
| (# particles/container) ^{a} | ≥25 | μm | N/A | N/A | N/A | 42 | N/A | 11 | 11 |
| MFI (# particles/container) | 2-300 | μm | 12061 | N/A | N/A | 12946 | N/A | 8967 | 11449 |
| (aspect ratio ≤0.85 applied) | 5-300 | μm | 1075 | N/A | N/A | 1010 | N/A | 528 | 638 |
| | 10-300 | μm | 445 | N/A | N/A | 414 | N/A | 184 | 200 |
| | 25-300 | μm | 13 | N/A | N/A | 16 | N/A | 2 | 7 |
| | 50-300 | μm | 0 | N/A | N/A | 0 | N/A | 0 | 0 |

^{a} Microscopy USP specifications: ≥10 μm sizes ≤3000 particles/container; ≥25 μm sizes ≤300 particles/container
HMW = high molecular weight;
LMW = low molecular weight;
N/A = not available

TABLE 28
Accelerated Storage and Thermal Stress Stability
Data for Formulation with 0.1% (w/v) PEG3350

| | | | | Length of Incubation | | | Length of Incubation | | |
|---|---|---|---|---|---|---|---|---|---|
| | | | | at 25° C. (months) | | | at 45° C.(months) | | |
| Assay | | | t = 0 | 1 | 3 | 6 | 0.5 | 1 | 2 |
| Purity by SE-UPLC | % HMW | | 2.0 | 2.7 | 3.0 | 3.8 | 3.7 | 4.8 | 8.6 |
| | % Main | | 97.0 | 97.1 | 97.0 | 95.0 | 95.3 | 93.7 | 88.9 |
| | % LMW | | 0.9 | 0.2 | 0.0 | 1.2 | 1.0 | 1.5 | 2.5 |
| Charge Variant Analysis | % Acidic | | 26.9 | 27.7 | 28.7 | 32.3 | 31.1 | 37.6 | 60.1 |
| by CEX-UPLC | % Main | | 58.2 | 57.1 | 55.5 | 53.6 | 55.4 | 48.5 | 26.2 |
| | % Basic | | 15.0 | 15.2 | 15.7 | 14.1 | 13.4 | 13.9 | 13.7 |
| Membrane Microscopy | ≥10 | μm | N/A | N/A | N/A | 27 | N/A | N/A | N/A |
| (# particles/container) a | ≥25 | μm | N/A | N/A | N/A | 2 | N/A | N/A | N/A |
| MFI (# particles/container) | 2-300 | μm | 12061 | N/A | N/A | 6177 | N/A | 3701 | N/A |
| (aspect ratio ≤0.85 applied) | 5-300 | μm | 1075 | N/A | N/A | 430 | N/A | 297 | N/A |
| | 10-300 | μm | 445 | N/A | N/A | 161 | N/A | 129 | N/A |
| | 25-300 | μm | 13 | N/A | N/A | 7 | N/A | 15 | N/A |
| | 50-300 | μm | 0 | N/A | N/A | 2 | N/A | 0 | N/A |

^{a} Microscopy USP specifications: ≥10 μm sizes ≤3000 particles/container; ≥25 μm sizes ≤300 particles/container
HMW = high molecular weight;
LMW = low molecular weight;
N/A = not available

TABLE 29
Agitation Stress Stability Data for Formulation with 0.1% (w/v) PEG3350

| | | | | | | | 24 hours | 24 hours | |
|---|---|---|---|---|---|---|---|---|---|
| | | | | | | | agitation*, | agitation*, | |
| | | | | | | | then storage | then storage | |
| | | | 24 hours agitation*, then | | | | at 25° C. | at 45° C. | |
| | | | storage at 5° C. (months) | | | | (months) | (months) | |
| Assay | | t = 0 | T0 | 6 | 12 | 18 | 6 | 1 | 2 |
| Purity by SE-UPLC | % HMW | 2.0 | 2.1 | 2.6 | 2.8 | 2.9 | 3.9 | 5.0 | 8.8 |
| | % Main | 97.0 | 97.9 | 96.4 | 96.5 | 96.3 | 95.0 | 93.7 | 88.9 |
| | % LMW | 0.9 | 0.0 | 1.0 | 0.7 | 0.9 | 1.1 | 1.3 | 2.3 |
| Charge Variant Analysis | % Acidic | 26.9 | 26.7 | 26.2 | 27.2 | 25.5 | 32.6 | 37.7 | 60.6 |
| by CEX-UPLC | % Main | 58.2 | 57.8 | 60.0 | 59.3 | 62.1 | 53.7 | 49.7 | 25.0 |
| | % Basic | 15.0 | 15.5 | 13.7 | 13.5 | 12.4 | 13.6 | 12.7 | 14.5 |

*Agitation for 24 hours at 25° C. followed by storage at indicated temperature
HMW = high molecular weight;
LMW = low molecular weight

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

TABLE 30
Sequences Excluded from ST.26-Formatted Sequence Listing

| SEQ ID NO: | Sequence |
|---|---|
| 7 | LGS |

1. A stable liquid pharmaceutical formulation comprising:
(i) a human antibody that specifically binds to human interleukin-4 receptor alpha (hIL-4Rα);
(ii) one or more buffers;
(iii) a thermal stabilizer;
(iv) a viscosity modifier; and
(v) a surfactant comprising a polyethylene glycol or a poloxamer at a concentration of from 0.01% w/v to 0.19% w/v,
wherein the formulation has a pH of from 5.7 to 6.1.

2. The stable liquid pharmaceutical formulation of claim 1, wherein the antibody comprises a heavy chain variable region (HCVR) comprising complementarity determining regions HCDR1, HCDR2, and HCDR3 comprising the amino acid sequences of SEQ ID NOs: 3, 4, and 5, respectively, and a light chain variable region (LCVR) comprising complementarity determining regions LCDR1, LCDR2, and LCDR3 comprising the amino acid sequences of SEQ ID NOs: 6, 7, and 8, respectively.

3. The stable liquid pharmaceutical formulation of claim 2, wherein the antibody comprises a HCVR comprising the amino acid sequence of SEQ ID NO: 1, and a LCVR comprising the amino acid sequence of SEQ ID NO: 2.

4. The stable liquid pharmaceutical formulation of claim 1, wherein the antibody has a human IgG heavy chain constant region.

5. The stable liquid pharmaceutical formulation of claim 4, wherein the heavy chain constant region is of isotype IgG1.

6. The stable liquid pharmaceutical formulation of claim 4, wherein the heavy chain constant region is of isotype IgG4.

7. The stable liquid pharmaceutical formulation of claim 1, wherein the antibody comprises a heavy chain comprising the amino acid sequence of SEQ ID NO: 9, and a light chain comprising the amino acid sequence of SEQ ID NO: 10.

8. The stable liquid pharmaceutical formulation of claim 1, wherein the surfactant is a polyethylene glycol.

9. The stable liquid pharmaceutical formulation of claim 8, wherein the polyethylene glycol is PEG3350.

10. The stable liquid pharmaceutical formulation of claim 1, wherein the surfactant is a poloxamer.

11. The stable liquid pharmaceutical formulation of claim 10, wherein the poloxamer is poloxamer 188.

12. The stable liquid pharmaceutical formulation of claim 1, wherein the buffer comprises an acetate buffer and/or a histidine buffer.

13. The stable liquid pharmaceutical formulation of claim 12, wherein the buffer comprises acetate at a concentration of from 10 mM to 15 mM.

14. The stable liquid pharmaceutical formulation of claim 13, wherein the acetate is present at a concentration of 12.5 mM±1.25 mM.

15. The stable liquid pharmaceutical formulation of claim 12, wherein the buffer comprises histidine at a concentration of from 15 mM to 25 mM.

16. The stable liquid pharmaceutical formulation of claim 15, wherein the histidine is present at a concentration of 20 mM±2 mM.

17. The stable liquid pharmaceutical formulation of claim 1, wherein the thermal stabilizer is sucrose at a concentration of from 2.5% w/v to 7.5% w/v.

18. The stable liquid pharmaceutical formulation of claim 17, wherein the sucrose is present at a concentration of 5% w/v±0.5% w/v.

19. The stable liquid pharmaceutical formulation of claim 1, wherein the viscosity modifier is arginine at a concentration of from 20 mM to 80 mM.

20. The stable liquid pharmaceutical formulation of claim 19, wherein the arginine is present at a concentration of 25 mM±2.5 mM.

21. The stable liquid pharmaceutical formulation of claim 19, wherein the arginine is present at a concentration of 50 mM±2.5 mM.

22. The stable liquid pharmaceutical formulation of claim 19, wherein the arginine is present at a concentration of 75 mM±2.5 mM.

23. The stable liquid pharmaceutical formulation of claim 1, wherein the antibody is present at a concentration up to 200 mg/mL.

24. The stable liquid pharmaceutical formulation of claim 1, wherein the antibody is present at a concentration of from 100 mg/mL to 200 mg/mL.

25. The stable liquid pharmaceutical formulation of claim 24, wherein the antibody is present at a concentration of 150 mg/mL±15 mg/mL.

26. The stable liquid pharmaceutical formulation of claim 24, wherein the antibody is present at a concentration of 175 mg/mL±15 mg/mL.

27. A stable liquid pharmaceutical formulation comprising:
(i) a human antibody at a concentration of 150 mg/mL±10 mg/mL, wherein the antibody specifically binds to human interleukin-4 receptor alpha (hIL-4Rα) and comprises a heavy chain variable region (HCVR) comprising the amino acid sequence of SEQ ID NO: 1, and a light chain variable region (LCVR) comprising the amino acid sequence of SEQ ID NO: 2;
(ii) acetate at a concentration of 12.5 mM±1.25 mM;
(iii) histidine at a concentration of 20 mM±2 mM;
(iv) sucrose at a concentration of 5% w/v±0.5% w/v;
(v) arginine at a concentration of 25 mM±2.5 mM; and
(vi) a surfactant comprising PEG3350 or poloxamer 188 at a concentration of from 0.01% w/v to 0.19% w/v,
wherein the formulation has a pH of 5.9±0.2.

28. A stable liquid pharmaceutical formulation comprising:
(i) a human antibody at a concentration of 175 mg/mL±10 mg/mL, wherein the antibody specifically binds to human interleukin-4 receptor alpha (hIL-4Rα) and comprises a heavy chain variable region (HCVR) comprising the amino acid sequence of SEQ ID NO: 1, and a light chain variable region (LCVR) comprising the amino acid sequence of SEQ ID NO: 2;
(ii) acetate at a concentration of 12.5 mM±1.25 mM;
(iii) histidine at a concentration of 20 mM 35 2 mM;
(iv) sucrose at a concentration of 5% w/v±0.5% w/v;
(v) arginine at a concentration of 25 mM±2.5 mM; and
(vi) a surfactant comprising PEG3350 or poloxamer 188 at a concentration of from 0.01% w/v to 0.19% w/v,
wherein the formulation has a pH of 5.9±0.2.

29. The stable liquid pharmaceutical formulation of claim 27, wherein the antibody comprises a heavy chain comprising the amino acid sequence of SEQ ID NO: 9, and a light chain comprising the amino acid sequence of SEQ ID NO: 10.

30. The stable liquid pharmaceutical formulation of claim 1, wherein the formulation contains a phospholipase protein.

31. The stable liquid pharmaceutical formulation of claim 1, wherein the formulation comprises no more than 3% high molecular weight (HMW) species following agitation by vortexing for up to 120 minutes at room temperature, as determined by size exclusion-ultra performance liquid chromatography (SE-UPLC).

32. (canceled)

33. The stable liquid pharmaceutical formulation of claim 1, wherein the formulation comprises no more than 8% high molecular weight (HMW) species after 30 days of storage at 45° C., as determined by SE-UPLC.

34-35. (canceled)

36. The stable liquid pharmaceutical formulation of claim 1, wherein the formulation comprises no more than 1000, no more than 500, or no more than 150 particles having a diameter of >10 μm in a volume of 2.25 mL after storage for 6 months at 5° C., as determined by microscopy, and wherein the formulation contains a phospholipase protein.

37. (canceled)

38. The stable liquid pharmaceutical formulation of claim 1, wherein the formulation comprises no more than 500, no more than 250, or no more than 50 particles having a diameter of >25 μm in a volume of 2.25 mL after storage for 6 months at 5° C., as determined by microscopy, and wherein the formulation contains a phospholipase protein.

39. (canceled)

40. The stable liquid pharmaceutical formulation of claim 36, wherein the phospholipase protein is phospholipase B-like 2 protein.

41. The stable liquid pharmaceutical formulation of claim 1, wherein the formulation comprises no more than 4% high molecular weight (HMW) species following agitation for 24 hours at 25° C., as determined by SE-UPLC.

42. (canceled)

43. The stable liquid pharmaceutical formulation of claim 1 contained in a glass vial.

44. The stable liquid pharmaceutical formulation of claim 1 contained in a syringe.

45. The stable liquid pharmaceutical formulation of claim 44, wherein the syringe comprises a fluorocarbon-coated plunger.

46. The stable liquid pharmaceutical formulation of claim 44, wherein the syringe is a low tungsten syringe.

47. The stable liquid pharmaceutical formulation of claim 44 that is a prefilled syringe.

48. The stable liquid pharmaceutical formulation of claim 47 that is a prefilled staked needle syringe.

49. The stable liquid pharmaceutical formulation of claim 1 contained in a large volume device or bolus injector.

50. A pen or autoinjector delivery device containing the stable liquid pharmaceutical formulation of claim 1.

51. The delivery device of claim 50 that is a disposable pen delivery device.

52. The delivery device of claim 50 that is a reusable pen delivery device.

53. A container containing the stable liquid pharmaceutical formulation of claim 1.

54. A kit comprising (i) a container containing the stable liquid pharmaceutical formulation of claim 1, and (ii) labeling for use of the pharmaceutical formulation.

55. The kit of claim 54, wherein the labeling recites subcutaneous administration of the pharmaceutical formulation.

56. (canceled)

57. A unit dosage form comprising the stable liquid pharmaceutical formulation of claim 1, wherein the antibody is present in an amount of from 1 mg to 500 mg.

58. The unit dosage form of claim 57, wherein the antibody is present in an amount of about 300 mg.

59. The unit dosage form of claim 57, wherein the antibody is present in an amount of about 200 mg.

60. The unit dosage form of claim 57, wherein the antibody is present in an amount of about 100 mg.

61. The unit dosage form of claim 57, wherein the formulation is contained in a syringe.

62. The unit dosage form of claim 61, wherein the syringe is a prefilled syringe.

63. A safety system delivery device containing a stable liquid pharmaceutical formulation of claim 1.

64-65. (canceled)
